SEQUENCE LISTING (6 sequences)

SEQ ID NO: 1 - protein, 6 aa, Artificial Sequence
  tetracysteine motif that binds ReAsH
CCPGCC

SEQ ID NO: 2 - DNA, 42 bp, Artificial Sequence
  primer
ctcgggatcgagggaaggatttcaagatataccatggacccc

SEQ ID NO: 3 - DNA, 30 bp, Artificial Sequence
  primer
gactctagaggatcctaggcacagcacgtg

SEQ ID NO: 4 - DNA, 32 bp, Artificial Sequence
  primer
gtgaccacatgtcacagcacgtgcacttgtcc

SEQ ID NO: 5 - DNA, 69 bp, Artificial Sequence
  primer
cgccctctagaaataattttgtttaactttaagaaggagatatacatatggatgagatggctaccactc

SEQ ID NO: 6 - DNA, 57 bp, Artificial Sequence
  primer
gccggatccctaaaccatggcacccgatccagaattaaacaggttagccacgaaagc

(19) United States
(12) Patent Application Publication
(10) Pub. No.: US 2009/0239215 A1
(43) Pub. Date: Sep. 24, 2009

(54) Clonable Tag for Purification and Electron Microscopy Labeling

(75) Inventors: David Derosier, Newton, MA (US); Christopher P. Mercogliano, Neptune City, NJ (US)

Correspondence Address: ROPES & GRAY LLP, PATENT DOCKETING 39/41, ONE INTERNATIONAL PLACE, BOSTON, MA, 02110-2624, US

(73) Assignee: BRANDEIS UNIVERSITY, Waltham, MA (US)

(21) Appl. No.: 11/793,282

(22) PCT Filed: Dec. 16, 2005
(86) PCT No.: PCT/US05/45926

§ 371 Date: Nov. 19, 2007

Related U.S. Application Data
(60) Provisional application No. 60/636,742, filed on Dec. 16, 2004.

(51) Int. Cl. G01N 33/68; C12Q 1/02; C12Q 1/68

(52) U.S. Cl. 435/6; 435/29; 435/40.5

(57) ABSTRACT

The disclosure provides compositions and methods for heavy atom labeling of a target protein using a clonable tag. The clonable tag comprises a metal binding protein, or fragment thereof, such as metallothionein, which may be fused to a target protein of interest. The tag permits the target protein to be labeled with a heavy atom, such as gold, silver, or mercury, and thus permits visualization of the target protein by electron microscopy. Also provided are methods for purification of a target protein using metallothionein, or a fragment thereof, as an affinity tag. The metallothionein fusion may be purified on immobilized metal affinity chromatography (IMAC) column charged with a metal such as cadmium.

### RELATED APPLICATIONS

This application claims the benefit of priority to U.S. Provisional Patent Application No. 60/636,742, filed on Dec. 16, 2004, which is hereby incorporated by reference in its entirety.

### STATEMENT REGARDING FEDERAL FUNDING

This invention was made with government support under Grant Numbers GM 26357, GM 35433, and GM 62580 awarded by the National Institutes of Health. The United States government has certain rights in the invention.

### BACKGROUND

The TEM has been a great source of data for various types of biological studies. Its greatest strength is the ability to resolve samples from the millimeter range down to the angstrom range. This means the TEM can resolve biological samples of cells within tissue through to the shapes of domains within individual proteins, and in recent years, down to even smaller parts such as protein backbones and large side chains. In this way the TEM is uniquely capable of linking the light microscopy studies of cell biology to the atomic resolution models of structural biology. Nevertheless, early biological work using the TEM mainly dealt with identifying and locating sub-cellular organelles and proteins within fixed cells since methods for extracting higher resolution information were unknown.

Although biological work using the TEM first involved only analysis of 2-dimensional images, the work of DeRosier and Klug in 1968 showed it was possible to perform 3-dimensional reconstruction of biological structures. This work noted that a 2-dimensional micrograph image corresponds to a projection of an object. Moreover, they outlined the central section theorem that states the Fourier transform of a projection is equivalent to a slice through the 3-dimensional Fourier space describing the object from which it was derived [1]. Therefore, if enough views, corresponding to varied angular slices of this 3-dimensional Fourier space are collected, it is possible to build up a 3-dimensional Fourier transform describing the object. Accordingly, a back transformation of this built up 3-dimensional Fourier transform can be performed to give a real space model of the object. In this way DeRosier and Klug were able to reconstruct the helically symmetric protein making up the T4 bacteriophage and were able to prove a general method for performing 3-dimensional electron microscopy reconstruction of biological samples [1]. Subsequent work by many groups through to the present has extended this method to near atomic resolution on some proteins [2] [3].

While most work performed in the field to date has concentrated on the high and low resolution extremes, perhaps the transmission electron microscope's greatest future potential is the marriage of these subfields in the form of electron tomography. Simply stated, electron tomography is the three dimensional reconstruction of cellular or large macromolecular biological samples at molecular resolutions. In electron tomography, tissue is chemically fixed or flash-frozen quickly enough to lock every molecule in the sample in a fixed position [4] [5]. This is thought to preserve all molecules with their interactions at the time of fixation. When placed in the TEM, a series of angular views will yield a 3-dimensional reconstruction. This method is akin to performing a CAT scan upon these samples, but instead of determining the different structures within a body, it is possible to determine the different structures within a single microscopic biological sample. When performed at the cellular level, this technique has the potential to allow biologist to observe proteins and their interactions in a cellular context [4].

However, the identification of specific proteins within images is problematic. All proteins are composed of material of nearly equal density, which do not vary much from other biological materials, essential ions, and water in the cellular milieu. As a result, TEM images of biological samples have low contrast. Added to this contrast issue are mechanical and physical limitations. Mechanically, it is impossible to collect large angular tilted views in the TEM. This lack of certain angular views, designated the missing cone, leads to an absence of information within the 3-dimensional Fourier transform. Furthermore, the low electron dose used to collect data without damaging the sample makes images from a tomographic data set extremely noisy [4]. Together, these limitations limit resolution to about 5 nanometers within tomograms and consequently make it impossible to identify all but the largest protein complexes by shape alone. Therefore, better methods for labeling proteins in TEM experiments are needed.

The earliest heavy metal labels used in TEM studies were for cellular histological work. Originally, large iron-rich ferritin complexes and colloidal gold particles (>5 nm) were adsorbed to primary antibodies for specific proteins or to secondary antibodies. This allowed for localization of proteins in tissue slices by the easy identification of the strongly scattering metal clusters within the low magnification electron microscope images [8]. However, localization and identification were limited since the labels were often larger than the proteins or complexes being studied. In addition, these clusters were located a length of an antibody molecule or two away from the protein of interest. Hence, this method is acceptable at a gross cellular level, but is often not precise enough for higher resolution work.

In order to deal with these resolution limiting issues, several smaller, commercial gold clusters have been developed for labeling protein complexes. Safer et al. introduced Undecagold® clusters in 1982 [9]. As its name suggests, this label contains eleven gold atoms. Notably, this label could not be seen directly in images, but rather it could only be observed in averaged images [10]. A decade later, a second larger gold cluster was introduced. Nanogold®, a 1.4 nm cluster, is believed to have between 55 and 75 gold atoms [11]. This cluster could be visualized directly in TEM images. Remarkably, Nanogold® has even been observed in images of heavy-metal stained proteins [12] [13]. The values of these cluster sizes yield the minimal numbers of atoms needed for useful TEM labels. Two important advantages of these labels can be attributed to their limited size. First, these clusters are often less detrimental to protein function as compared to larger antibody-based labels. More importantly, within numerous studies of biological complexes, Undecagold® and Nanogold® clusters have allowed enhanced precision for localization of proteins of interest.

Perhaps as important as size for providing better precision by these commercial labels is their mode of attachment to a protein of interest. In contrast to traditional, indirect labeling methods used to label proteins of interest, commercial labels are superior because they can be attached directly to a protein of interest rather than through a labeled antibody bound to an epitope. Thus, these commercial metal clusters can be chemically attached to the protein of interest without the aid of a secondary protein. Commercial gold clusters can be so attached because they are surrounded by an organic shell that can be modified with a monofunctional reactive group. Consequently, clusters can be covalently affixed to a specific type of amino acid in a protein of interest [14]. Initially, the thiol groups of a protein's cysteines were targeted by a maleimide group on the surface of an Undecagold® cluster [10]. The short length of these covalent tethers more precisely localizes a labeling site within a protein complex. In addition, these shorter tether lengths limit the freedom of movement of clusters at labeling sites. This aids identification within 3-dimensional reconstructions by reinforcing the contribution of clusters to a smaller set of voxels within the averaged structure. Thus, this distinct combination of characteristics has made these commercial gold clusters the benchmark for TEM localization studies of macromolecular complexes.

In the last several years, an alternative type of TEM label has been developed for cellular level labeling. This method involves the non-fluorescent biarsenical fluorescein derivative, ReAsH, that can bind a genetically engineered tetracysteine motif. The optimal tetracysteine motif has been determined to be Cys-Cys-Pro-Gly-Cys-Cys (SEQ ID NO: 1), and it has been suggested to form a hairpin structure when ReAsH binds [15]. Once bound, the ReAsH-tetracysteine complex can fluoresce at a red wavelength of 608 nm, and it can be used for light microscopy [16]. Another secondary application of this motif is its ability to act as a purification tag. For this purpose, a sister biarsenical fluorescein derivative to ReAsH, FlAsH, is coupled to an agarose support matrix and allows for affinity purification of tetracysteine tagged proteins [15]. Hence, this label is multifunctional.

To function as a TEM label, cells with ReAsH-labeled proteins must be glutaraldehyde fixed and perfused with DAB, diaminobenzidine. Then ReAsH can be used to photoconvert molecular oxygen to singlet oxygen. This singlet oxygen will swiftly react with DAB causing it to locally polymerize and precipitate. Once enough DAB has been reacted, the cells must then be stained with osmium tetroxide. This stain strongly binds to the precipitated and polymerized DAB and provides the electron density that acts as the TEM label [16]. The size and shape of the stained electron-dense material is variable, relying on the photo-oxidation process. Published results using this method often show sizable regions of labeling rather than isolated, individually labeled proteins [17] [16]. The advantage of this method over conventional cellular labeling techniques is the specificity and efficiency of this label. A disadvantage is that this method functions only in fixed tissue. However, perhaps of more concern, is the local creation of oxygen radicals which may react and damage the protein of interest. Thus, this method is well suited for moderate resolution but may not function for higher-resolution cellular studies.

While commercial clusters are valuable tools, this does not mean they work perfectly. Perhaps the greatest challenge when labeling a protein of interest is label specificity. Even in small proteins, there are likely to be more than one copy of any of the 20 biological amino acids. Hence, metal clusters directed to specific amino acid types may label proteins at multiple sites. This means additional work will be required to identify the corresponding sites of attachment [18]. When dealing with large macromolecular complexes where tens or hundreds of labeling sites may be possible, this issue may make direct labeling to specific amino acid types a labor intensive obstacle rather than a useful technique. A second consequence of multiple labeling sites is that with each additional label, there is an increased chance of disturbing protein function and structure [18]. Although it may be possible to circumvent these issues with a variety of techniques, these specificity issues regularly make the process of labeling an art rather than a straightforward method.

In recent years, specificity of TEM labels has been increased by further modifying the organic shells of clusters. Fusion of small molecules onto the surfaces of clusters can allow for strong specific binding to non-covalent sites formed by proteins. The fused moieties include small molecules, such as ATP, which can be directed to active sites in protein complexes [19]. Even more impressive is the addition of a metal affinity matrix molecule. In this case, a tetradentate nitrilotriacetic acid (NTA) group charged with nickel can direct clusters to a hexa-histadine recombinant tag on a protein of interest [20]. Although these non-covalent labels show increased specificity since they require binding sites of several amino acids, their use can still be challenging. The added volume of the cluster can affect the interaction of these small molecules with their corresponding sites. Alternatively, the added volume may hinder penetration of clusters into deeply buried binding sites within complexes of interest [19]. This highlights that labeling efficiency is equally important to specificity when attempting to label complexes.

Two additional impediments, which can commonly hinder labeling, have to do with the chemical composition of these commercial labels. One job of the organic shells of these labels is to form a protective coat around the gold cluster. This can occasionally result in binding of the label to surfaces other than those expected on a protein of interest. In this way, the label non-specifically localizes to the protein [19], [11]. In addition, the connections of the organic shell to the label's metal core are chemically labile. As a result, certain chemicals, especially strong reducing agents, can strip the organic shell rendering the label non-functional [11]. Hence, there is no magic technique that will work universally, and additional modes of labeling can always be of use.

With the new interest in cellular electron tomography, the need for alternative labeling methods has gained new importance. The common method for labeling chemically fixed tissue involves washing labeled antibodies over tissue slices. Unfortunately, labeling efficiency is often poor with labeling only accruing at the surfaces of tissue slices [21]. This most likely results from the relatively larger size of antibody molecules that consequently makes them difficult to perfuse through the tissue [8]. Furthermore, the fixation process can alter cellular surfaces and as such may inhibit the efficiency of binding of these antibodies [21]. Interpretation of antibody labeling in cellular environments is difficult as described earlier due to the proximity of label to proteins of interest. Moreover, no method currently exists for labeling proteins within whole cells without first disrupting cell membranes to introduce labels. This is sub-optimal when studying flash-frozen, unfixed tissue samples using higher-resolution cellular electron tomography since preservation of native structures is desired. Accordingly, the development of new TEM labels is needed to overcome these specificity and efficiency issues in a cellular context.

Labeling in light microscopy of cells had been beleaguered by many of the same issues now apparent in cellular electron tomography. However, these were overcome through the use of recombinant DNA technologies and the development of clonable labels such as green fluorescent protein (GFP). By genetically fusing GFP to proteins of interest, complete, specific labeling can be performed, and proteins can be localized in cells. A comparable technique for genetically fusing a TEM label to a protein of interest would be highly advantageous for use in both high resolution TEM of protein complexes and cellular electron tomography.

### SUMMARY

We have now discovered that metallothionein may be used as a clonable tag for purification and heavy atom labeling of a target protein. Metallothionein is a small protein that can bind a variety of metal atoms, such as, for example, gold atoms. Like green fluorescent protein (a tag used for light microscopy), metallothionein can be used to create a fusion protein with a target protein. The ability of metallothionein to bind gold permits a gold cluster to be assembled directly on the fusion protein and does not require the introduction of preformed gold clusters into cells.

Additionally, we have developed a method for purifying a target protein using metallothionein as an affinity tag in conjunction with an immobilized metal affinity column charged with metal atoms such as cadmium.

### BRIEF DESCRIPTION OF THE DRAWINGS

FIG. 1 shows the structure of metallothionein. (A) Shows the overall dumbbell-shaped domain structure of metallothionein. The backbone (blue) wraps around the two metal clusters. (B) and (C) show the close up views of the beta domain (residues 1-30) and alpha domain (residues 31-61), respectively. The sulfurs (yellow) of the cysteines coordinate the various metal atoms. In this panel B, the 9 cysteines of the beta domain coordinate 1 cadmium atom (red) and 2 zinc atoms (silver). Likewise in panel C, the 11 cysteines of the alpha domain coordinate 4 cadmium atoms. This figure was generated with Rasmol using the atomic coordinates (4MT2) deposited by Braun et al (1992).

FIG. 2 shows the chemical structures for several gold containing anti-arthritic drugs. The chemical structures of the three common gold(I) anti-arthritic drugs are shown with their molecular weights. Each compound performs similar chemistry that can be explained by their similar binding of gold (yellow) through a thiolate bond with the single sulfur in each structure. Both aurothiomalate and aurothioglucose form polymers in solution while auranofin does not. The extra phosphine ligand (PEt₃) attached to the gold in auranofin blocks the formation of bridging ligands with reactive groups in other molecules.

FIG. 3 shows the results of ESI mass spectrometry of metal bound metallothionein. The raw mass spectra (A, C, and E in the left column) and mass deconvoluted spectra (B, D, and F in the right column) are plotted as percent intensity verse mass-to-charge. Spectra A and B are from apo-metallothionein. Spectra C and D are from Zn₇-metallothionein. Spectra E and F are from aurothiomalate incubated metallothionein with a 1 to 1 ratio of gold to metallothionein's cysteines. In A, C, and E the peaks are labeled as ‘M’ those resulting from monomers and ‘D’ for those resulting from dimers. The charge associated with each peak is listed in parentheses. In B, D and F the mass value for the peak maximum is listed above each peak.

FIG. 4 shows Table 1 containing the expected mass values for zinc containing Metallothionein. This table contains mass values for metallothionein containing different numbers of zinc atoms. The value is calculated by the formula:

Expected Mass=(Mₐₚₒ₋ₘₑₜₐₗₗₒₜₕᵢₒₙₑᵢₙ)+{(# zinc)*(M_{zinc})}−{(# zinc)*{(3*M_{hydrogen})/(# zinc)}}.

FIG. 5 shows Table 2 containing expected mass values for gold containing metallothionein. This table contains mass values for metallothionein containing different numbers of gold atoms. The value is calculated by the formula:

Expected Mass=(Mₐₚₒ₋ₘₑₜₐₗₗₒₜₕᵢₒₙₑᵢₙ)+{(# gold)*(M_{gold})}−{(# gold)*{(3*M_{hydrogen})/(# gold)}}.

FIG. 6 shows Table 3 containing expected mass values for aurothiomalate containing metallothionein. This table contains mass values for metallothionein containing different numbers of aurothiomalate (AuStm) molecules. The value is calculated by the formula:

Expected Mass=(Mₐₚₒ₋ₘₑₜₐₗₗₒₜₕᵢₒₙₑᵢₙ)+{(# AuStm)*(M_{AuStm})}−{(# AuStm)*{(3*M_{hydrogen})/(# AuStm)}}.

FIG. 7 shows the results of MALDI mass spectrometry of gold bound metallothionein. This figure displays the MALDI mass spectra results for several incubations of aurothiomalate with metallothionein. All spectra are plotted as percent intensity versus mass to charge. Panel A is the spectrum of apo-metallothionein. Panel B is the spectrum of a sample incubated at a ratio of 1 to 1 of aurothiomalate with metallothionein's cysteines. Panels C and D show spectra of samples incubated at a ratio of 10 to 1 of aurothiomalate to metallothionein's cysteines. The values printed to the right of each peak are the mass to charge ratio values corresponding to the maximum intensity witnessed for that peak. In each case, both the monomer and dimer peaks have been listed. The monomer peak values show 0, 19, 30, and 33 gold atoms bound in A, B, C, and D, respectively.

FIG. 8 shows an enlarged view of MALDI mass spectra of gold-incubated metallothioneins. Panels A and B show close-up views of two aurothiomalate-incubated metallothionein samples resulting in low and high gold capacity binding, respectively. In each, a strong periodicity is observed. The maximum peaks in A and B correspond to 15 and 30 gold atoms, respectively.

FIG. 9 shows fourier transforms of mass spectra. The Fourier transforms for the spectra in FIG. 8 are shown in A and B, respectively. The arrows point to the peaks resulting from the high frequency periodicities observed in FIG. 8. The values of 0.00517 Hz and 0.00485 Hz correspond to peak to peak wavelengths of 193.4 amu and 206.2 amu, respectively.

FIG. 10 shows TEM images of metallothionein. To visualize metallothionein with and without gold bound, mass spectrometry samples were placed on a very thin carbon foils supported upon Quantifoil® TEM grids. Panels E show a metallothionein sample viewed at 94,000× at the edge of a carbon covered hole, and Panels A, B, C, D, and F shows samples viewed at 250,000× on the thin carbon foil. Panels A and C are images from control samples containing buffer and aurothiomalate at the sample concentration as the gold-incubated metallothionein samples in Panels E and F. Panel C shows the occasionally witnessed large aggregates believed to be undissolved aurothiomalate. Panel B is a control sample prepared from a sample of buffer with no protein. Panel D is from a sample of Zn₇-metallothionein with no gold. Notice that Panel D has a light modulation of the background suggesting the presence of sample material. Panels E and F display the highly visible electron dense particles believed to be gold bound metallothionein. A variation in size is witnessed, possibly due to aggregates of gold bound protein

FIG. 11 shows the separation of MBP-Metallothionein fusion by size exclusion chromatography. Typical elution profiles collected on a Pharmacia Superdex1030HR column for the various two MBP-metallothionein fusion proteins with gold (blue) and without gold (red) monitoring UV absorbance at 280 nm are shown. For comparison, aurothiomalate (green) elute later than protein peaks. The ‘scaled’ designation refers to the 2 to 3 times increase in absorption at 280 nm of equal protein concentration samples containing gold. MBP-metallothionein fusions show a characteristic series of peaks suggestive of oligomerization. With gold, both proteins elute more quickly from the column. The buffer was 100 mM ammonium acetate pH 6.

FIG. 12 shows the absorption changes in gold-labeled MBP Metallothionein Fusion proteins. Absorption spectra were collected to compare changes resulting from gold binding after sizing column separation. As controls, samples of aurothiomalate (blue) and Nanogold® (black) were also examined. MBP-MT2 protein incubated with gold (green) shows increased absorption values between 240 nm and 400 nm as compared to MBP-MT2 without gold (red). Notably, MBP-MT2 and Nanogold® contain an extended shoulder at wavelengths greater than 300 nm, but the Nanogold® shoulder extends much further than the 400 nm cutoff seen for MBP-MT2 incubated with gold.

FIG. 13 shows mass spectrometry verification of sizing column fraction composition. To evaluate the exact composition of size exclusion column fractions, samples were subjected to MALDI mass spectrometry. Panel A shows the spectrum resulting from MBP-MT protein from a monomer peak fraction. Notably, the main mass spectrometry peak is consistent with a monomer state. A small dimer peak and extremely weak trimer peak are also present. Panel B shows the spectrum collected for MBP-MT protein from a trimer fraction. Although monomer signal is observed, a relatively more intense trimer peak is observed as compared to panel A. Maximum mass peak values are printed to the right of each of the peaks with their corresponding charge values in parentheses.

FIG. 14 shows mass spectrometry verification of gold binding to the MBP-MT Fusion Protein. Evaluation of the ability of the MBP-MT protein to bind gold from aurothiomalate was performed by collecting MALDI mass spectra. Panel A shows the apo-MBP-MT protein. Likewise, Panel B shows the spectrum collected for the aurothiomalate incubated MBP-MT sample. Maximum mass peak values are printed to the right of peaks with their corresponding charge values in parentheses. As expected, an increase in mass peak value and peak distribution for the gold-incubated sample is observed.

FIG. 15 shows mass spec verification of gold binding to the MBP-MT2 Fusion Protein. Evaluation of the ability of the MBP-MT2 protein to bind gold from aurothiomalate was performed by collecting MALDI mass spectra. Panel A shows the apo-MBP-MT2 protein. Likewise, Panel B shows the spectrum collected for the aurothiomalate incubated MBP-MT2 sample. Maximum mass peak values are printed to the right of peaks with their corresponding charge values in parentheses. Again, an expected increase in mass peak value and peak distribution for the gold-incubated sample was observed.

FIG. 16 shows STEM and TEM imaging of MBP-MT Fusion Proteins. STEM images (left column) and TEM images (right column) taken without staining show small, nanometer or small electron dense clusters in sample of MBP-MT incubated with gold versus those incubated with no metal. The no metal MBP-MT images (A and B) only show all occasional smear of density from protein alone (yellow arrow). In E and F, the gold-bound MBP-MT protein is shown. The red circles show examples of what are believed to be single gold MBP-MT clusters. These are smaller than the Nanogold® (blue squares) images shown in C and D.

FIG. 17 shows STEM and TEM imaging of MBP-MT2 Fusion Proteins. STEM images (left column) and TEM images (right column) taken without staining show about 1.4 nm electron dense clusters in sample of MBP-MT2 incubated with gold versus those incubated with no metal. The no metal MBP-MT2 images (A and B) only show an occasional smear of density from protein alone (yellow arrow). In E and F, the gold-bound MBP-MT2 protein is shown. The red circles show examples of what are believed to be single gold MBP-MT2 clusters. These are at times larger than the Nanogold® (blue squares) cluster images as shown in C and D.

FIG. 18 shows STEM and TEM images of trimerized MBP-MT2 Fusion Protein. Evaluation of the trimer fractions of MBP-MT2 protein prove useful in discerning the size of individual concatenated metallothionein gold clusters. Panels A and B show only weak scattering from protein alone (yellow arrow). Examples of the gold incubate MBP-MT2 proteins are circled in red. The STEM image in panel C shows well separated strongly scattering aggregates about 3 times the size observed for MBP-MT2 monomers in FIG. 17. However, panel D shows distinct, well-separated aggregates with 2 to 3 electron-dense clusters. Since these samples are from trimer fractions, the individual clusters are likely single gold clusters formed by one copy of MBP-MT2.

FIG. 19 shows the results of a separation of MBP-MT2-Antibody Complex. In order to insure imaging involves only formed antibody complex, incubated samples were separated on a Pharmacia Superose 12 column. The elution profiles for MBP-MT2 alone (blue), MBP antibody alone (black), and two different complex formation reactions (red and green) are shown. Comparing the profiles of the high MBP-MT2 to antibody ratio run (red) to the low MBP-MT2 to antibody ratio run (green) shows the development of a second peak in the high ratio sample run. This second peak elutes at the same location as MBP-MT2, and suggests saturation of antigen binding sites.

FIG. 20 shows a gallery of Antibody and Antibody Complexes viewed in Stain TEM Images. To evaluate the peak antibody complex elution fraction from the size exclusion column before preparing cryo-TEM grids, the protein was view with 2% uranyl acetate stain. Anti-MBP antibody with no antigen is shown in the upper 15 images while the antibody complex sample is displayed in the lower 15 images. On average, particles from the antibody complex sample (lower 15 images) appear larger. Especially noticeable is the extra mass present on the ends of two of the antibody complex domains as compared to naked antibody.

FIG. 21 shows a gallery of cryo-electron microscopy images of Antibody Complexes. Micrographs of the antibody complex collected under low dose conditions were inspected for the characteristic Y-shaped view. In ice, complexes are randomly oriented so that observing these views is rare. The gallery of images in this figure show five examples of the y-shaped particles believed to be antibody complex formed with aurothiomalate incubated MBP-MT2. The sketches below each image are presented to aid visualizing the complexes in the noisy, low contrast images.

FIG. 22 shows a cryo-EM image of gold bound MBP-MT2. Gold-labeled MBP-MT2 protein was used to prepared cryo-TEM grids. Images were taken at 75,000× on a Philips CM12 TEM using low dose conditions. The blue circles contain examples of electron dense clusters at their centers. These clusters appear about the same size as Nanogold®.

FIG. 23 shows a gallery of STEM images of Antibody Complexes. To better evaluate metallothionein's role as a TEM label, antibody complexes formed with gold-labeled (bottom) and unlabeled (top) MBP-MT2 were imaged by STEM. The images are noisy and difficult to interpret. Therefore, the sketch below each image is provided as an aid for observing the imaged complex. The arrow indicates a region of strong scattering suggestive of gold cluster formation using metallothionein. The lack of gold attributable signal in some of the gold-labeled MBP-MT2-antibody complex may suggest antigen binding sites are not completely occupied.

FIG. 24 shows a distribution of STEM mass measurements. This histogram was created using the limited amount of STEM data. Mass measurements were calculated from the combined information from the high and low annular detectors of the STEM. PCMass25, a computer program written and distributed by Joseph Wall at Brookhaven national Laboratories was used to obtain values. In addition, an average of 205.2 kDa with a standard deviation of 44.7 was calculated. This may indicate that all antigen bind sited are not occupied in these samples.

FIG. 25 shows the restoration of gold-incubated RecA function by Penicillamine. A 1000 base pair piece of DNA was used to assess nucleoprotein complex formation with RecA. A mobility shift assay performed within a 0.8% agarose gel with subsequent ethidium bromide staining allowed for efficient monitoring this reaction. Lane 1 shows a significantly slower mobility of DNA bound within these nucleoprotein complexes as compared to DNA alone (Lane 6). Lane 2 shows the less well defined and more mobile band resulting for prior gold labeling of RecA with aurothiomalate, a gold(I) compound, indicating inhibition of protein function. Lanes 2, 3, and 4 show the reestablishment of wild-type function with increased concentrations of penicillamine within reaction mixtures

FIG. 26 shows a MALDI mass spectra of aurothiomalate-incubated RecA protein. Mass spectra of RecA were collected to observe mass shifts resulting from gold(I) binding via incubation with aurothiomalate as well as the removal of the additional mass via incubation with penicillamine. All spectra contain peaks corresponding to protein monomers with a +1 and +2 charge as designated in parentheses within the figure. Maximum peak mass-to-charge values are written to the right of each peak. Panel A is a control sample displaying the observed mass of the wild-type RecA. Panel B shows the development of a second peak at a higher mass value with the subsequent relative decrease in the peak intensity corresponding to the wild-type RecA protein alone. Panel C displays almost a complete return of the observed peak maximum to a wild-type value upon incubation with 10 mM penicillamine.

FIG. 27 shows a MALDI mass spectra of Penicillamine incubated gold bound Metallothionein. Mass spectra were collected to evaluate penicillamine's ability to strip metal atoms from gold-bound metallothionein. Panel A is a negative control displaying the spectrum of apo-metallothionein. Panel B shows a positive control of gold-bound metallothionein with a peak indicating the high gold-binding state. Panel C shows the spectrum observed after incubation with 20 mM penicillamine. Although this peak shows slightly less mass at its peak value and tightening of the mass distribution, the gold-bound metallothionein still shows a high degree of metal binding with about 27 gold atoms bound. Panel D is another control sample containing only aurothiomalate. The gold compound appears responsible for the sharp series of peaks below a mass-to charge ratio of about 5000 amu in B and D.

FIG. 28 shows a conventional His-tagged affinity purification of a Metallothionein fusion protein. A kinesin fusion protein fused to metallothionein, a biotinylation tag, and a hexa-histidine tag was purified using a conventional nickel-bound immobilized metal column. (Left) This is an SDS-PAGE 12% gel showing fairly specific isolation of the kinesin fusion protein overexpressed in E. coli. (Right) Gel displaying a western for an identically loaded gel to the Coomassie gel using an anti-hexa-histidine direct antibody developed using a horse radish peroxidase development system.

FIG. 29 shows the binding and elution of Zn₇-Metallothionein to metal affinity columns. Nickel, zinc, and cadmium charged columns, as well as an uncharged column, were tested for their ability to bind zinc-bound metallothionein. A sample of Zn₇-Metallothionein was loaded into each of these columns, rinsed, and eluted using EDTA. Samples of fraction were loaded into SDS-PAGE gels and visualized with Coomassie stain. The results are seen in these two gels with the type of column tested listed above the corresponding lanes. Control samples of Zn₇-Metallothionein not placed through a column were included (control). Only the cadmium charged column showed binding and elution.

FIG. 30 shows a deconvoluted ESI mass spectra of cadmium column purified metallothionein. The eluted protein from the cadmium column in FIG. 25 was assessed for its metal content as it was eluted. All spectra are mass deconvoluted, corresponding to their zero charge state, with peak mass values listed to the right of the peak. Panel A shows a negative control of apo-metallothionein. Panel B is a positive control displaying the spectrum of the zinc-bound metallothionein that was loaded onto the cadmium column. Panel C is the spectrum collected for the metallothionein containing sample eluted from the cadmium column. A distinct increase in mass to 6912 amu is observed.

FIG. 31 shows the results of affinity purification using Metallothionein as an isolation tag. This gel shows the affinity purification of a Fimbrin N375 protein construct genetically fused to metallothionein using the developed cadmium column affinity purification technique. Samples from the various fractions were denatured and run on a 12% SDS-PAGE gel. After electrophoresis, the gel was rinsed in 20% methanol and then stained with a solution of 20% methanol containing 100 μM monobromobimane, a cysteine modifying reagent, for 30 minutes. After staining the gel was rinse and visualized on a standard UV light box using a green filter (image shown on right). The gel was then stain using a Coomassie stain (image shown on left). An intense single band of kinesin-metallothionein is eluted from the column.

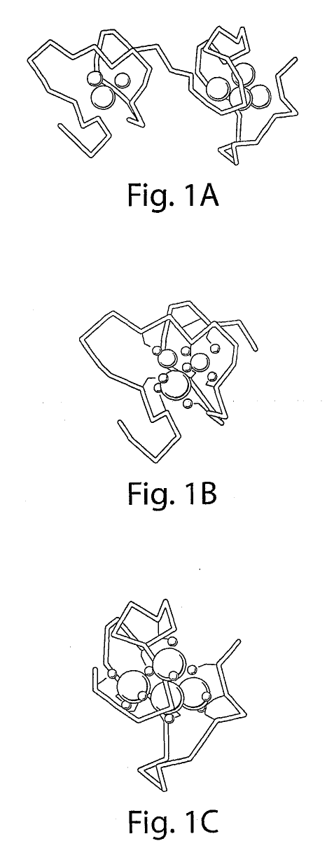

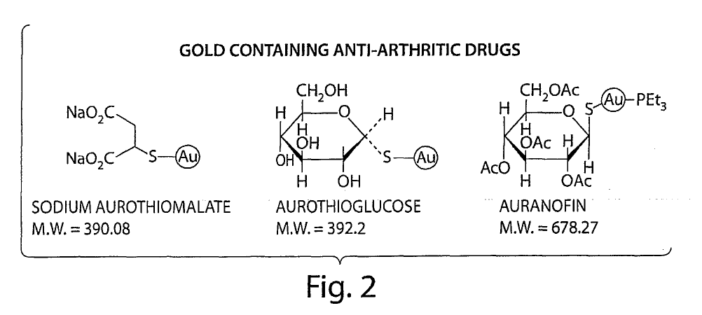

### DETAILED DESCRIPTION

“Coding sequence” is used herein to refer to the portion of a nucleic acid that encodes a particular protein. A coding region may be interrupted by introns and other non-coding sequences that are ultimately removed prior to translation.

“Colloidal suspension” is used herein to refer to a colloidal suspension that comprises one or more nucleic acids for delivery to cells. The material in a colloidal suspension is generally designed so as to protect nucleic acids and facilitate the delivery of nucleic acids across cell membranes. Exemplary colloidal suspensions include, but are not limited to, lipid micelles, tubes, rafts, sandwiches and other lipid structures, often comprising cationic lipids. Other colloidal suspensions include nanocapsules, microbeads and small, nucleic acid-binding polymeric structures, etc.

An “externally regulated promoter” is a nucleic acid that affects transcription in response to conditions that may be provided in a controlled manner by one of skill in the art. Externally regulated promoters may be regulated by specific chemicals, such as tetracycline or IPTG, or by other conditions such as temperature, pH, oxidation state etc. that are readily controlled external to the site of transcription.

“Homology” or “identity” or “similarity” refers to sequence similarity between two polypeptides or between two nucleic acid molecules. Homology and identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology/similarity or identity refers to a function of the number of identical or similar amino acids at positions shared by the compared sequences. A sequence which is “unrelated” or “non-homologous” shares less than 40% identity, though preferably less than 25% identity with a sequence of the present invention.

The term “homology” describes a mathematically based comparison of sequence similarities which is used to identify genes or proteins with similar functions or motifs. The nucleic acid and protein sequences of the present invention may be used as a “query sequence” to perform a search against public databases to, for example, identify other family members, related sequences or homologs. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and BLAST) can be used. See the world wide web at ncbi.nlm.nih.gov.

As used herein, “identity” means the percentage of identical nucleotide or amino acid residues at corresponding positions in two or more sequences when the sequences are aligned to maximize sequence matching, i.e., taking into account gaps and insertions. Identity can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987, and Sequence Analysis Primer, Gribskov, M. and Devereux, J., eds., M Stockton Press, New York, 1991; and Carillo, H., and Lipman, D., SIAM J. Applied Math., 48: 1073 (1988). Methods to determine identity are designed to give the largest match between the sequences tested. Moreover, methods to determine identity are codified in publicly available computer programs. Computer program methods to determine identity between two sequences include, but are not limited to, the GCG program package (Devereux, J., et al., Nucleic Acids Research 12(1): 387 (1984)), BLASTP, BLASTN, and FASTA (Altschul, S. F. et al., J. Molec. Biol. 215: 403-410 (1990) and Altschul et al. Nuc. Acids Res. 25: 3389-3402 (1997)). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990).

As used herein, the term “nucleic acid” refers to polynucleotides such as deoxyribonucleic acid (DNA), and, where appropriate, ribonucleic acid (RNA). The term should also be understood to include analogs of either RNA or DNA made from nucleotide analogs (including analogs with respect to the base and/or the backbone, for example, peptide nucleic acids, locked nucleic acids, mannitol nucleic acids etc.), and, as applicable to the embodiment being described, single-stranded (such as sense or antisense), double-stranded or higher order polynucleotides.

The term “operably linked” is used herein to refer to the relationship between a regulatory sequence and a gene. If the regulatory sequence is positioned relative to the gene such that the regulatory sequence is able to exert a measurable effect on the amount of gene product produced, then the regulatory sequence is operably linked to the gene.

A “polylinker” is a nucleic acid comprising at least two, and preferably three, four or more restriction sites for cleavage by one or more restriction enzymes. The restriction sites may be overlapping. Each restriction sites is preferably five, six, seven, eight or more nucleotides in length.

A “recombinant helper nucleic acid” or more simply “helper nucleic acid” is a nucleic acid which encodes functional components that allow a second nucleic acid to be encapsidated in a capsid. Typically, in the context of the present invention, the helper plasmid, or other nucleic acid, encodes viral functions and structural proteins which allow a recombinant viral vector to be encapsidated into a capsid. In one preferred embodiment, a recombinant adeno-associated virus (AAV) helper nucleic acid is a plasmid encoding AAV polypeptides, and lacking the AAV ITR regions. For example, in one embodiment, the helper plasmid encodes the AAV genome, with the exception of the AAV ITR regions, which are replaced with adenovirus ITR sequences. This permits replication and encapsidation of the AAV replication defective recombinant vector, while preventing generation of wild-type AAV virus, e.g., by recombination.

A “regulatory nucleic acid” or “regulatory sequence” includes any nucleic acid that can exert an effect on the transcription of an operably linked open reading frame. A regulatory nucleic acid may be a core promoter, an enhancer or repressor element, a complete transcriptional regulatory region or a functional portion of any of the preceding. Mutant versions of the preceding may also be considered regulatory nucleic acids.

A “transcriptional fusion” is a nucleic acid construct that causes the expression of an mRNA comprising at least two coding regions. In other words, two or more open reading frames may be organized into a transcriptional fusion such that both open reading frames will be expressed as part of a single mRNA and then give rise, as specified by the host cell, to separate polypeptides. The open reading frames in a transcriptional fusion tend to be subject to the same transcriptional regulation, but the encoded polypeptides may be subject to distinct post-translational fates (eg. degradation, etc.). A “transcriptional fusion” may be contrasted with a “translational fusion” in which two or more open reading frames are connected so as to give rise to a single polypeptide. The fused polypeptides in a “translational fusion” tend to experience similar transcriptional, translational and post-translational regulation.

As used herein, the term “transfection” means the introduction of a nucleic acid, e.g., an expression vector, into a recipient cell, and is intended to include commonly used terms such as “infect” with respect to a virus or viral vector. The term “transduction” is generally used herein when the transfection with a nucleic acid is by viral delivery of the nucleic acid. “Transformation”, as used herein, refers to a process in which a cell's genotype is changed as a result of the cellular uptake of exogenous DNA or RNA, and, for example, the transformed cell expresses a recombinant form of a polypeptide or, in the case of anti-sense expression from the transferred gene, the expression of a naturally-occurring form of the recombinant protein is disrupted.

As used herein, the term “transgene” refers to a nucleic acid sequence which has been introduced into a cell. Daughter cells deriving from a cell in which a transgene has been introduced are also said to contain the transgene (unless it has been deleted). A transgene can encode, e.g., a polypeptide, partly or entirely heterologous, i.e., foreign, to the transgenic animal or cell into which it is introduced. Optionally, a transgene-encoded polypeptide may be homologous to an endogenous gene of the transgenic animal or cell into which it is introduced, but may be designed to be inserted, or is inserted, into the genome in such a way as to alter the genome of the cell into which it is inserted (e.g., it is inserted at a location which differs from that of the natural gene). Alternatively, a transgene can also be present in an episome. A transgene can include one or more transcriptional regulatory sequences and any other nucleic acid, (e.g. intron), that may be necessary for optimal expression of a selected coding sequence. A transgene may also contain no polypeptide coding region, but in such cases will generally direct expression of a functionally active RNA, such as an rRNA, tRNA, ribozyme, etc. A “therapeutic transgene” is a transgene that is introduced into a cell, tissue and/or organism for the purpose of altering a biological function in a manner that is beneficial to a subject.

“Transient transfection” refers to cases where exogenous nucleic acid is retained for a relatively short period of time, often when the nucleic acid does not integrate into the genome of a transfected cell, e.g., where episomal DNA is transcribed into mRNA and translated into protein. A cell has been “stably transfected” with a nucleic acid construct comprising viral coding regions when the nucleic acid construct has been introduced inside the cell membrane and the viral coding regions are capable of being inherited by daughter cells.

“Viral particle” is an assemblage of at least one nucleic acid and a coat comprising at least one viral protein. In general, viral particles for use in delivering nucleic acids to cells will retain the ability to insert the nucleic acid into a cell, but may be defective for many other functions, such as self-replication.

### Exemplary Methods

Provided herein is a clonable tag for purification and heavy atom labeling of proteins. The tag is a polypeptide that is capable of binding to one or more heavy atoms, such as, for example, metallothionein or a fragment thereof. The tag may be attached to any target protein of interest using standard recombinant DNA techniques. The fusion construct may be expressed using an in vitro system or by introduction of the fusion construct into a cell (eukaryotic or prokaryotic). The fusion construct may be contained on a vector that permits transient or stable transfection of the cell.

The clonable tag permits very high efficiency labeling of the target protein. Presently available tags have only a very low efficiency of labeling, such that only about 5% of a target protein will be labeled with gold. The tag provided herein will permit at least about 10%, 20%, 50%, 75%, 80%, 90%, 95%, 99%, or more of a target protein to be labeled.

Also provided are methods for labeling cells without disrupting the cellular membrane of the cell. For example, a cell containing an expression construct for a target protein-metallothionein fusion protein may be contacted with gold containing compounds that can diffuse across the cell membrane, such as, for example, aurothiomalate, aurothioglucose, or auranofin. Alternatively, heavy metal compounds that are not capable of traversing the cell membrane may be used in combination with a technique for cell permeabilization such as, for example, electroporation or treatment with detergent. Exemplary heavy metal compounds include, for example, gold compounds such as Nanogold or Undecagold. In yet another embodiment, cells may be modified so as to increase their ability to import heavy metals, such as gold, into the cell. For example, a cell may be modified so as to contain one or more genes from the mer operon, such as, merA, merC, merD, merP, merR, and/or merT. See e.g., Summers A O, Sugarman L I. Cell-free mercury(II)-reducing activity in a plasmid-bearing strain of Escherichia coli. J. Bacteriol. 1974 119(1):242-9; Hamlett N V, et al., Roles of the Tn21 merT, merP, and merC gene products in mercury resistance and mercury binding. J. Bacteriol. 1992 174(20):6377-85; Park S J, et al., Genetic analysis of the Tn21 mer operator-promoter. J. Bacteriol. 1992 April; 174(7):2160-71; and Liebert, C. A., et al. Tn21, flagship of the floating genome. Microbiol. Mol. Biol. Rev. 63 (3), 507-522 (1999).

In one embodiment, a method for examination of a target protein over time is provided. A cell expressing a fusion polypeptide comprising the target protein and clonable tag (such as metallothionein) is contacted with different heavy atoms at different time points over the lifetime of the cell or cell culture. For example, at one time point the cell may be contacted with a gold compound which binds very tightly to metallothionein. At later time point, the cell is contacted with a silver compound which binds to metallothionein but not tightly enough to displace gold that is already bound to the metallothionein. The gold bound vs. silver bound fusion proteins may be distinguished by electron microscopy and the gold bound compounds would represent copies of the target protein that were expressed at an earlier time point and the silver bound copies of the target protein would represent copies of the target protein that were expressed at a later time point.

In another embodiment, a method for examining two or more different proteins by electron microscopy are provided. Different target proteins may be fused to different numbers of tandem repeats of the clonable tag. The greater the number of repeats of the clonable tag, the greater the number of gold atoms that can be bound by the target protein fusion thus producing a stronger signal on electron microscopy. For example, target protein A may be fused to a single copy of metallothionein (or a fragment thereof) while target protein B may be fused to two or more tandem repeats of metallothionein (or a fragment thereof). Upon contact with a heavy metal, such as gold, target protein B should bind greater numbers of gold atoms thus producing a stronger EM signal.

Metallothionein proteins have been cloned from a wide variety of organisms and their sequences may be found in various publicly available databases such as GenBank (world wide web at ncbi.nlm.nih.gov). Exemplary metallothionein genes include, for example, those from human (GenBank Accession No. NM-005946 (nucleotide), NP_{—}005937 (protein)); mouse (GenBank Accession No. NM_{—}013602 (nucleotide), NP_{—}038630 (protein)); rat (GenBank Accession No. NM_{—}138826 (nucleotide), NP_{—}620181 (protein)); and rabbit (GenBank Accession No. X07791 (nucleotide), S54334 (protein)). Fragments of metallothionein proteins may also be suitable for use as a clonable EM tag, such as, for example, fragments comprising the alpha domain of metallothionein.

### Electron Microscopy

A TEM image is a recording of the point-to-point variation of the electron wavefront being passed through a sample. Depending on the materials present at different positions in the sample, electrons will interact more or less strongly with the atoms of the sample. This creates variation in the collected wavefront that will ultimately form the TEM image. Thus, contrast in a TEM image depends directly upon the spatial variations and scattering factors of atoms within a sample.

In the TEM, scattering of electrons by a sample is mainly through electrostatic interactions. These mainly arise from the negative charges of the TEM beam electrons interacting with the negatively charged electrons or positively charged atomic nuclei of the sample. In this way, negatively charged sample electrons deflect the beam by the repulsive forces of like charges, and positively charged atomic nuclei deflect the beam through attractive forces as the beam electrons pass through the sample. However, since sample electrons are diffused in orbits around their atoms, the attractive deflections resulting from the large atomic centered positive nuclear charges are much more prevalent [6]. Additionally, the interactions with nuclei with large nuclear charges, such as gold and other heavy metals, will scatter far better than the lower atomic numbered atoms of biological samples. As a rule of thumb, the scattering ability of an atom is approximately Z^{2/3} power, where Z is the atom's atomic number [7].

Although scattering power is significant, given the limits of resolution in the TEM, the number and arrangement of atoms in a label are also important. In the best TEM reconstructions, which are around 4 Å resolution, any 3-dimensional pixel, known as a voxel, represents a 2 Å cube of the sample. This space would only represent about 1 atom. Applying the Z^{2/3} power rule, if the atom was carbon, the scattering ability of this region of space would be (6^{2/3}≈3.3 or 3.3/8 Å³≈0.41/Å³). On the other hand, if this atom were exchanged with a gold atom, the scattering ability would be (79^{2/3}≈18.4 or 18.4/8 Å³≈2.3/Å³). Hence, without accounting for other limitations, a single heavy atom, even in the best reconstruction, will only yield about 5 times signal increase in a voxel. However, this becomes much worse as the voxel size increases. By 8 Å resolution, where each voxel represents a 4 Å cube, a single gold atom in a volume with 7 carbon atoms yields a value of only 0.65/Å³ versus 0.41/Å³ for the same voxel with 8 carbon atoms. Thus, many closely packed atoms are needed to increase signal. Metals, such as gold, are well suited for this purpose since these atoms like to make metal-metal bonds and as such form tightly packed clusters. This semi-crystalline structure increases the scattering effect due to the high-density atomic packing within a cluster. In this way, packing is as vital as scattering in TEM label design.

### Metallothionein

Metallothioneins encompass a vast family of proteins. First reported in horse liver in 1957, they were further examined due to their sizeable metal and sulfur contents [30]. Of particular interest was the presence of the biologically toxic metal, cadmium, bound to the protein [31]. Subsequent work over the next two decades showed that metallothioneins were present within numerous vertebrates and invertebrates. Most often, the protein was found in the detoxifying organs, namely the liver and kidney, but also metallothionein expression has been found in many other tissue types [32]. Additional work showed that metallothioneins are expressed in plants, most often within the roots, and even within unicellular eukayotes, such as Neurospora crassa [32] and Saccharomyces cerevesiae [33]. With the exception of Saccharomyces cerevesiae, all of these versions appear evolutionarily descended from a common ancestor and comprise Class I metallothioneins [32] [34]. Saccharomyces cerevesiae and prokaryotes, such as the sewage sludge bacteria, Pseudomonas putida, and the cyanobacteria, Synechococcus, [35] [36] have metallothionein-like proteins. However, these versions are most likely derived from convergent evolution and comprise Class II metallothioneins [32] [34]. A third class contains metallothionein-like proteins such as the enzymatic-concatenated peptide, phytochelatin. Regardless of class, the environmental and tissue specific locations of expression led to the early belief that metallothioneins had purely a metal detoxification role.

Through early studies, several common features of metallothioneins were noted. These features include: high metal content bound by thiolate bonds, a high cysteine content (usually 23-33%), a molecular weight below 10,000 Daltons, and a structure similar to the mammalian protein [32]. In this work, rabbit liver metallothionein-1 and mouse metallothionein-1 have been used in experiments. The subgroup distinction, 1 through 4, of mammalian metallothioneins designates the copy of the gene that is located within a single gene cluster [34]. However, both rabbit liver and mouse metallothionein-1 sequences and functions are almost identical to all other mammalian versions.

The primary protein sequences and compositions of mammalian metallothioneins are highly conserved. Typically, the sequences are composed of 60-62 amino acids with 20 of these being cysteines. Overall, the sequence hints at a gene duplication with the first thirty amino acids weakly mirrored in the second thirty [32]. The cysteines are found with the consensus sequences of Cys-Cys, Cys-X-Cys, Cys-X—X-Cys, or Cys-X-Cys-Cys [37]. These cysteine motifs form the basic unit for metal atom coordination in the protein. Thus, these cysteines are the source of metallothionein's ability to bind 5-7 metal atoms or 10-12 metal atoms with a positive 2 or 1 charge, respectively [32].

Other amino acids are also over represented in the sequence. Specifically, arginine and lysine generally make up 14% of the sequence. These residues often are found adjacent to the cysteines and are believed to neutralize the charge of the metal thiolate ligands [38]. Also, proline is found invariantly at about position 38 or 39 in all vertebrates sequences as well as providing the defining metallothionein-2 subgroup when located in positions 10 or 11. Interestingly, there are few aromatic residues, and no histidines in the mammalian metallothioneins. Other than the characteristic cysteines, these amino acid preferences are only well conserved in the mammalian homologues, but do not appear as common in more distantly related eukaryotic or prokaryotic homologues.

Most notably, the secondary and tertiary structures of metallothionein highlight the proteins uniqueness. The protein's structure was determined first by x-ray crystallography and later by NMR [37]. Structures for the 12 atom monovalent cation case and the 7 atom divalent cation case have been determined, but higher metal content forms of the protein have not been reported. The structures show the protein folds into a dumbbell shaped structure with two domains, alpha (residues 31-61) containing 11 cysteines and beta (residues 1-30) containing 9 cysteines. Each domain binds metal atoms as a cluster surround by the polypeptide chain. This orients the cysteines of each cluster inward towards the metal atoms. Consequently, each domain lacks a hydrophobic core found in most other proteins.

The simplest description of the secondary structure is that it does not possess one. The only semblance of true secondary structure is a short alpha 3/10 helix seen at residues 41 to 47 in some x-ray crystal structures, but this feature is absent in NMR models. Instead, an early non-conventional secondary structural interpretation by Furey et al. claimed each domain has four beta strands that organize into an anti-parallel beta sandwich using cysteine-metal-cysteine bonds in place of the common beta sheet hydrogen bonds [39]. Although this interpretation is questionable, it does highlight the metal atom's overwhelming role in guiding protein folding. Nevertheless, the consensus is that the backbone is two long loops forming each domain.

Although the mammalian metallothionein gene was most likely formed via a gene duplication, the domains' structures are conspicuously dissimilar. The alpha domain has 11 cysteines and binds 4 divalent or 7 monovalent cations. Conversely, the beta domain has only 9 cysteines and binds 3 divalent or 5 monovalent cations. The alpha domain wraps around its metal cluster in a left-handed fashion while the beta domain wraps around its cluster in a right-handed manner. Although this was noted by several structural studies, no functional significance has been hypothesized nor have significant contacts between the two domains been observed. In addition, elongation of the linker region (residues 30 to 32) between the two domains with up to 16 amino acids does not alter in vivo function [40]. These and other results led to the conclusion that there was little communication between the two domains. However, cadmium binding studies of independently isolated domains showed the alpha domain can fold and bind metal atoms without the beta domain, but not vice versa [41]. Later, independent site-directed mutagenesis of each domain's cysteines to alanine again showed that alpha domain metal binding was needed for beta domain metal binding [42]. While this contradicts earlier results where isolated alpha and beta domains were able to bind metal atoms identically to their manner in full length protein [43], it highlights the possibility that protein function could be controlled primarily by the alpha domain.

Further metal binding regulation may be attributed to metallothionein's quaternary structure and non-metallic biological ligands. In the crystal structure, metallothionein molecules are associated in dimers mediated by phosphates or sodium atoms [37]. Dynamic light-scattering studies suggest that these cysteine-independent dimers as well as higher order polymers are present in solution under certain conditions [44]. This dimerization may help trap metals within the protein.

Other non-metallic biological ligands such as glutathione, glutathione disulphide, and ATP may interact with metallothionein. Moreover, their interactions highlight metallothionein functions as more than a metal scavenger. Normally in a cell, metallothionein binds seven zinc atoms, which is a fairly harmless metal [32]. At cellular concentrations of glutathione, it has been hypothesized that metallothionein is found in a partially open confirmation where two glutathione molecules are bound to help protect the zinc atoms [45]. Upon oxidative stress, whether caused by invasion of redox active metals or not, glutathione disulphide builds up within cells. This molecule can oxidize metallothionein's cysteines. Upon oxidation of only a few of metallothionein's cysteines, zinc atoms can be released [46]. This event has two consequences. First, the remaining cysteines become available for use as an antioxidant, and any redox active metals with higher affinities than zinc that are immediately available become sequestered. Second, the zinc release causes up regulation of metallothionein transcription, which is under control of metal regulatory element [32]. Hence, metallothionein can act to protect the redox environment inside a cell.

The role of ATP is unclear and still debated. No ATP-binding site is identifiable in the protein sequence, but an association with a sub-millimolar binding constant has been detected [45]. Some research groups suggest this is merely a weak electrostatic interaction observed under non-physiologic conditions [47]. A key justification for binding was an increased transfer rate of zinc from metallothionein to apo-sorbital dehydrogenase in the presence ATP [45]. Although this does not prove ATP is a cellular ligand, it does demonstrate metallothionein's possible role in shuttling zinc within cells.

Unlike the metal binding sites of most proteins, which show a strict preference for atoms of particular elements in specific ionic states, metallothioneins bind a variety of metals in a range of valence states. To accommodate these diverse metals, metallothionein must bind atoms with various coordination numbers. Given the periodic table of elements and metallothionein's metal affinities, the protein's metal binding sites prefers larger, softer metal atoms. This preference makes chemical sense given the soft thiolate ligands of the 20 cysteines responsible for metal binding [43]. These cysteines can bind as either terminal or bridging ligands [39]. However, this simple metal preference view is complicated by valence state.

Given the concentration and number of cysteines within the protein, reactions of metal atoms to lower, more reduced states are possible and sometimes necessary for stable complex formation. For example, copper in aqueous solution is most often found in the divalent form. For metallothionein binding, copper(II) is reduced to copper(I) and then bound. This is due to the relatively smaller reduction potential (+330 mV) between copper's two ionic states as compared to metallothionein redox potential of −366 mV [46]. This reduction potential places metallothionein as having one of the greatest redox potentials within cells [46]. Moreover, this further highlights metallothionein's function as an anti-oxidant.

The most commonly bound metal in vivo is zinc, and it is bound with a thermodynamic stability constant, K_{d}=1.4×10⁻¹³ molar at pH 7.0 [46]. Although this is quite strong, it represents one of the weakest stability constants for metal-metallothionein complexes. The stability constants for most metals have not been directly measured, but rather they have been inferred by proton titration studies of the various metal-metallothionein complexes. While 50 percent of zinc bound to metallothionein is released at pH 4.8, metals such as bismuth, mercury, palladium and platinum show great binding stability and are not removed even below pH 1.8 [43]. Hence, binding to these larger, softer metals can be considered covalent.

Further pH-dependence of metal binding is apparent during protein folding. NMR data suggest apo-metallothionein is a flexible polypeptide chain [48]. During binding by divalent cobalt, the first few atoms bind independently at non-specific cysteine motifs, but binding becomes cooperative upon addition of the fourth atom at pH 7.2 or fifth atom at pH 8.4. This cooperativity co-develops with adjustment of metal atoms into the alpha domain [49]. Previously, similar pH effects were obtained with cadmium, however the cooperativity onset was witnessed with fewer metal atoms. Good et al. stated, “The observed pH dependence of cluster formation in MT can be rationalized by the degree of deprotonation of cysteine residues (pKₐ approximately 8.9), i.e., by the difference in Gibbs free energy required to bind Cd(II) ions to thiolate ligands at both pH values [48].” Therefore, the energy released by deprotonation, which increases with decreasing pH, provides the energy needed for folding metallothionein's domains into clusters. Furthermore, this provides a basis for understanding the energetics of metallothionein cluster formation.

Aurothionein is a complex of gold-metallothionein. These complexes are formed though reactions of gold containing anti-arthritic drugs, such as aurothiomalate, aurothioglucose, and auranofin. Furthermore, these complexes can form both in vivo and in vitro [50-52]. In vitro, gold can displace either zinc or cadmium from metallothionein in a minimally metal-dependent rate. These reactions occur within tens of minutes when performed in stoichiometric ratios [53]. Additionally, metallothionein reacted with excess molar ratios of anti-arthritic compounds can bind as many as 20 gold atoms. However, it should be noted that the organic portions of these anti-artritic drug molecules may still remain bound to gold within these metallothionein complexes [52].

Like gold(I), both silver(I) and mercury(II) react with metallothionein to form complexes with large metal-to-metallothionein ratios. In both cases, stable 18 metal atom structures form [54, 55, 56]. Extended X-ray absorption fine structure (EXAFS) studies on the mercury complex suggest metal atoms are bridged between metallothionein sulfurs with no additional ligands. Hence, this led to the hypothesis that these 18 metal atom metallothionein complexes refold into a single domain [57]. Gold(I) should be chemically similar to both silver(I) and mercury(II), which are one row higher in the same periodic table group and isoelectronic to gold(I), respectively. Thus, this suggests gold bound to metallothionein may also adopt a single cluster structure, and as such gold-metallothionein complexes may function as electron dense labels.

### Heavy Atoms

The methods and compositions described herein may use a variety of heavy atom labels that are suitable for use as a label for electron microscopy. In exemplary embodiments, suitable heavy atoms include, for example, gold (Au), Silver (Ag), mercury (Hg), cadmium (Cd), zinc (Zn), platinum (Pt), bismuth (Bi), or combinations thereof.

The elemental qualities of gold are the basis for cluster formation and make it ideal for TEM labels. The first step in gold cluster formation is the reduction and subsequent coordination of gold(III) to gold(I) with either thiol or phosphine ligands. This stabilizes these atoms as gold(I) and drives them to prefer a linear coordination. Thus, a polymer of gold(I) atoms bridged between either a series of thiols or phosphines develops. Upon addition of excess reducing agent, some of the gold(I) is further reduced to gold(0). Gold(0) is hydrophobic and likes to make metal-metal bonds. Hence, gold(0) and gold(I) atoms condense into clusters with their thiol or phosphine ligands forming a monolayer organic shell [22] [11]. Although these cores are described as having gold(0) atoms at their centers with gold(I) atoms layering their outer regions, atoms appear to assume a close-packed semi-crystalline structure where electrons are thought to be delocalized throughout [22]. For clusters of forty or fewer gold atoms, this leads to significant spectroscopic effects in the visible and near-UV regions [23] [22]. However, these properties become more like those of bulk metal as the clusters grow [24].

In an exemplary embodiment, the methods and compositions described herein may employ a gold containing compound that is capable of diffusing across a cell membrane, such as, for example, aurothiomalate, aurothioglucose, or auranofin. Alternatively, the gold sources that cannot diffuse across the cell membrane may be used in conjuction with a cell permeabilizing technique such as, for example, electroporation or chemical treatment with, for example, a detergent. Other examples of suitable gold sources include undecagold and nanogold clusters.

In order to increase the visibility of a gold cluster bound to the clonable tag, the clusters can be used as catalysts for a developer containing silver ions. Silver precipitates around the cluster and this newly precipitated silver itself catalyzes further reduction of silver ions to metallic silver. By this process, with the gold cluster acting as a nucleation center, silver particles may be grown to almost any desired size by controlling the reaction time, temperature and other parameters. In this way, the formerly “invisible” gold cluster may now be visualized in commercial electron microscopes, in light microscopes, and with large silver grains, with the unaided eye. A number of silver developers are known in the literature and are available commercially that deposit silver around gold metal (e.g., Nanoprobes, Inc., Yaphank, N.Y.; world wide web at nanoprobes.com).

### Constructs and Vectors

In certain aspects, the disclosure provides vectors and nucleic acid constructs comprising nucleic acids encoding one or more proteins that may be used as a clonable tag for electron microscopy. In an exemplary embodiment, nucleic acid constructs encoding a fusion polypeptide comprising a target protein and at least one copy of a clonable tag for electron microscopy are provided. The clonable tag may be metallothionein, tandem repeats of metallothionein, a fragment of metallothionein, or tandem repeats of a fragment of metallothionein. The clonable tag may comprise, for example, two, three, four, five, or more tandem repeats of metallothionein, or a fragment thereof. The nucleic acid constructs may also optionally encode for a linker between the target protein and the clonable tag and/or a linker between tandem repeats of the clonable tag. The linker may be, for example, a short polypeptide sequence comprising, for example, 2-50 amino acids, 2-30 amino acids, 2-20 amino acids, or 2-10 amino acids. In certain embodiments, it may be desirable to incorporate one or more charged amino acid residues into the linker.

Other features of the vector or construct will generally be designed to supply desirable characteristics depending on how the clonable tag is to be generated and used. Exemplary desirable characteristics include but are not limited to, gene expression at a desired level, gene expression that is reflective of the expression of a different gene, easy clonability, transient or stable gene expression in subject cells, etc.

In certain aspects, it is desirable to use a vector that provides transient expression of the clonable tag. Such vectors will generally be unstable inside a cell, such that the nucleic acids necessary for expression of the clonable tag are lost after a relatively short period of time. Optionally, transient expression may be effected by stable repression. Exemplary transient expression vectors may be designed to provide gene expression for an average time of hours, days, weeks, or perhaps months. Often transient expression vectors do not recombine to integrate with the stable genome of the host. Exemplary transient expression vectors include: adenovirus-derived vectors, adeno-associated viruses, herpes simplex derived vectors, hybrid adeno-associated/herpes simplex viral vectors, influenza viral vectors, especially those based on the influenza A virus, and alphaviruses, for example the Sinbis and semliki forest viruses.

In some aspects the invention provides a vector or construct comprising a readily clonable nucleic acid encoding a clonable tag. For example, the coding sequence may be flanked by a polylinker on one or both sides. Polylinkers are useful for allowing one of skill in the art to readily insert the coding sequence in a variety of different vectors and constructs as required. In another example, the coding sequence may be flanked by one or more recombination sites. A variety of commercially available cloning systems use recombination sites to facilitate movement of the desired nucleic acid into different vectors. For example, the Invitrogen Gateway™ technology utilizes a phage lambda recombinase enzyme to recombine target nucleic acids with a second nucleic acid. Each nucleic acid is flanked with appropriate lambda recognition sequence, such as attL or attB. In other variations, a recombinase such as topoisomerase I may be used with nucleic acids flanked by the appropriate recognition sites. For example, the Vaccinia virus topoisomerase I protein recognizes a (C/T)CCTT sequence. These recombination systems permit rapid shuffling of flanked cassettes from one vector to another as needed. A construct or vector may include both flanking polylinkers and flanking recombination sites, as desired.

In certain aspects, the clonable tag, or a fusion between a clonable tag and target protein, is operably linked to a promoter. The promoter may for example, be a strong or constitutive promoter, such as the early and late promoters of SV40, or adenovirus or cytomegalovirus immediate early promoter. Optionally it may be desirable to use an externally regulated promoter, such as a tet promoter, IPTG-regulated promoters (GAL4, Plac), or the trp system. In view of this specification, one of skill in the art will readily identify other useful promoters depending on the downstream use. For example, the invention may utilize exemplary promoters such as the T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast a-mating factors, the polyhedron promoter of the baculovirus system and other sequences known to control the expression of genes of prokaryotic or eukaryotic cells or their viruses, and various combinations thereof. In addition, as noted above, it may be desirable to have a clonable tag, or a fusion between a clonable tag and target protein, operably linked to a promoter that provides useful information about the condition of the cell in which it is situated.

Vectors of the invention may be essentially any nucleic acid designed to introduce and/or maintain a clonable tag, or a fusion between a clonable tag and target protein, in a cell or virus. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) may be used.

### Nucleic Acids for Delivery to Organisms and In Vitro Tissues

Instead of ex vivo modification of cells, in many situations one may wish to modify cells in vivo. For this purpose, various techniques have been developed for modification of target tissue and cells in vivo. A number of viral vectors have been developed, such as described above, which allow for transfection and, in some cases, integration of the virus into the host. See, for example, Dubensky et al. (1984) Proc. Natl. Acad. Sci. USA 81, 7529-7533; Kaneda et al., (1989) Science 243, 375-378; Hiebert et al. (1989) Proc. Natl. Acad. Sci. USA 86, 3594-3598; Hatzoglu et al. (1990) J. Biol. Chem. 265, 17285-17293 and Ferry, et al. (1991) Proc. Natl. Acad. Sci. USA 88, 8377-8381. The vector may be administered by injection, e.g. intravascularly or intramuscularly, inhalation, or other parenteral mode. Non-viral delivery methods such as administration of the DNA via complexes with liposomes or by injection, catheter or biolistics may also be used.

In general, the manner of introducing the nucleic acid will depend on the nature of the tissue, the efficiency of cellular modification required, the number of opportunities to modify the particular cells, the accessibility of the tissue to the nucleic acid composition to be introduced, and the like. The DNA introduction need not result in integration. In fact, non-integration often results in transient expression of the introduced DNA, and transient expression is often sufficient or even preferred.

Any means for the introduction of polynucleotides into mammals, human or non-human, may be adapted to the practice of this invention for the delivery of the various constructs of the invention into the intended recipient. In one embodiment of the invention, the nucleic acid constructs are delivered to cells by transfection, i.e., by delivery of “naked” nucleic acid or in a complex with a colloidal dispersion system. A colloidal system includes macromolecule complexes, nanocapsules, microspheres, beads, and lipid-based systems including oil-in-water emulsions, micelles, mixed micelles, and liposomes. An exemplary colloidal system of this invention is a lipid-complexed or liposome-formulated DNA. In the former approach, prior to formulation of DNA, e.g., with lipid, a plasmid containing a transgene bearing the desired DNA constructs may first be experimentally optimized for expression (e.g., inclusion of an intron in the 5′ untranslated region and elimination of unnecessary sequences (Felgner, et al., Ann NY Acad Sci 126-139, 1995). Formulation of DNA, e.g. with various lipid or liposome materials, may then be effected using known methods and materials and delivered to the recipient mammal. See, e.g., Canonico et al, Am J Respir Cell Mol Biol 10:24-29, 1994; Tsan et al, Am J Physiol 268; Alton et al., Nat. Genet. 5:135-142, 1993 and U.S. Pat. No. 5,679,647 by Carson et al.

Optionally, liposomes or other colloidal dispersion systems are targeted. Targeting can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs, which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

The surface of the targeted delivery system may be modified in a variety of ways. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand. A certain level of targeting may be achieved through the mode of administration selected.

In certain variants of the invention, the nucleic acid constructs are delivered to cells, and particularly cells in an organism or a cultured tissue, using viral vectors. The transgene may be incorporated into any of a variety of viral vectors useful in gene therapy, such as recombinant retroviruses, adenovirus, adeno-associated virus (AAV), herpes simplex derived vectors, hybrid adeno-associated/herpes simplex viral vectors, influenza viral vectors, especially those based on the influenza A virus, and alphaviruses, for example the Sinbis and semliki forest viruses, or recombinant bacterial or eukaryotic plasmids. The following additional guidance on the choice and use of viral vectors may be helpful to the practitioner.

### Herpes Virus Systems

A variety of herpes virus-based vectors have been developed for introduction of genes into mammals or mammalian cells. For example, herpes simplex virus type 1 (HSV-1) is a human neurotropic virus of particular interest for the transfer of genes to the nervous system. After infection of target cells, herpes viruses often follow either a lytic life cycle or a latent life cycle, persisting as an intranuclear episome. In most cases, latently infected cells are not rejected by the immune system. For example, neurons latently infected with HSV-1 function normally and are not rejected. Some herpes viruses possess cell-type specific promoters that are expressed even when the virus is in a latent form.

A typical herpes virus genome is a linear double stranded DNA molecule ranging from 100 to 250 kb. HSV-1 has a 152 kb genome. The genome may include long and short regions (termed UL and US, respectively) which are linked in either orientation by internal repeat sequences (IRL and IRS). At the non-linker end of the unique regions are terminal repeats (TRL and TRS). In HSV-1, roughly half of the 80-90 genes are non-essential, and deletion of non-essential genes creates space for roughly 40-50 kb of foreign DNA (Glorioso et al, 1995). Two latency active promoters which drive expression of latency activated transcripts have been identified and may prove useful for vector transgene expression (Marconi et al, 1996).

HSV-1 vectors are available in amplicons and recombinant HSV-1 virus forms. Amplicons are bacterially produced plasmids containing OriC, an Escherichia coli origin of replication, OriS (the HSV-1 origin of replication), HSV-1 packaging sequence, the transgene under control of an immediate-early promoter & a selectable marker (Federoff et al, 1992). The amplicon is transfected into a cell line containing a helper virus (a temperature sensitive mutant) which provides all the missing structural and regulatory genes in trans. More recent amplicons include an Epstein-Barr virus derived sequence for plasmid episomal maintenance (Wang & Vos, 1996). Recombinant viruses are made replication deficient by deletion of one the immediate-early genes e.g. ICP4, which is provided in trans. Deletion of a number of immediate-early genes substantially reduces cytotoxicity and allows expression from promoters that would be silenced in the wild type latent virus. These promoters may be of use in directing long term gene expression. Replication-conditional mutants replicate in permissive cell lines. Permissive cell lines supply a cellular enzyme to complement for a viral deficiency. Mutants include thymidine kinase (During et al, 1994), ribonuclease reductase (Kramm et al, 1997), UTPase, or the neurovirulence factor g34.5 (Kesari et al, 1995). These mutants are particularly useful for the treatment of cancers, killing the neoplastic cells which proliferate faster than other cell types (Andreansky et al, 1996, 1997). A replication-restricted HSV-1 vector has been used to treat human malignant mesothelioma (Kucharizuk et al, 1997). In addition to neurons, wild type HSV-1 can infect other non-neuronal cell types, such as skin (Al-Saadi et al, 1983), and HSV-derived vectors may be useful for delivering transgenes to a wide array of cell types. Other examples of herpes virus vectors are known in the art (U.S. Pat. No. 5,631,236 and WO 00/08191).

### Adenoviral Vectors

A viral gene delivery system useful in the present invention utilizes adenovirus-derived vectors. Knowledge of the genetic organization of adenovirus, a 36 kB, linear and double-stranded DNA virus, allows substitution of a large piece of adenoviral DNA with foreign sequences up to 8 kB. In contrast to retrovirus, the infection of adenoviral DNA into host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage. In addition, adenoviral vector-mediated transfection of cells is often a transient event. A combination of immune response and promoter silencing appears to limit the time over which a transgene introduced on an adenovirus vector is expressed.

Adenovirus is particularly suitable for use as a gene transfer vector because of its mid-sized genome, ease of manipulation, high titer, wide target-cell range, and high infectivity. The virus particle is relatively stable and amenable to purification and concentration, and as above, can be modified so as to affect the spectrum of infectivity. Additionally, adenovirus is easy to grow and manipulate and exhibits broad host range in vitro and in vivo. This group of viruses can be obtained in high titers, e.g., 10⁹-10¹¹ plaque-forming unit PFU)/ml, and they are highly infective. Moreover, the carrying capacity of the adenoviral genome for foreign DNA is large (up to 8 kilobases) relative to other gene delivery vectors (Berkner et al., supra; Haj-Ahrmand and Graham (1986) J. Virol. 57:267). Most replication-defective adenoviral vectors currently in use and therefore favored by the present invention are deleted for all or parts of the viral E1 and E3 genes but retain as much as 80% of the adenoviral genetic material (see, e.g., Jones et al., (1979) Cell 16:683; Berkner et al., supra; and Graham et al., in Methods in Molecular Biology, E. J. Murray, Ed. (Humana, Clifton, N.J., 1991) vol. 7. pp. 109-127). Expression of the inserted polynucleotide of the invention can be under control of, for example, the E1A promoter, the major late promoter (MLP) and associated leader sequences, the viral E3 promoter, or exogenously added promoter sequences.

The genome of an adenovirus can be manipulated such that it encodes a gene product of interest, but is inactivated in terms of its ability to replicate in a normal lytic viral life cycle (see, for example, Berkner et al., (1988) BioTechniques 6:616; Rosenfeld et al., (1991) Science 252:431-434; and Rosenfeld et al., (1992) Cell 68:143-155). Suitable adenoviral vectors derived from the adenovirus strain Ad type dl324 or other strains of adenovirus (e.g., Ad2, Ad3, Ad7 etc.) are well known to those skilled in the art.

Adenoviruses can be cell type specific, i.e., infect only restricted types of cells and/or express a transgene only in restricted types of cells. For example, the viruses may be engineered to comprise a gene under the transcriptional control of a transcription initiation region specifically regulated by target host cells, as described e.g., in U.S. Pat. No. 5,698,443, by Henderson and Schuur, issued Dec. 16, 1997. Thus, replication competent adenoviruses can be restricted to certain cells by, e.g., inserting a cell specific response element to regulate a synthesis of a protein necessary for replication, e.g., E1A or E1B.

DNA sequences of a number of adenovirus types are available from Genbank. For example, human adenovirus type 5 has GenBank Accession No. M73260. The adenovirus DNA sequences may be obtained from any of the 42 human adenovirus types currently identified. Various adenovirus strains are available from the American Type Culture Collection, Rockville, Md., or by request from a number of commercial and academic sources. A transgene as described herein may be incorporated into any adenoviral vector and delivery protocol, by restriction digest, linker ligation or filling in of ends, and ligation.

Adenovirus producer cell lines can include one or more of the adenoviral genes E1, E2a, and E4 DNA sequence, for packaging adenovirus vectors in which one or more of these genes have been mutated or deleted are described, e.g., in PCT/US95/15947 (WO 96/18418) by Kadan et al.; PCT/US95/07341 (WO 95/346671) by Kovesdi et al.; PCT/FR94/00624 (WO94/28152) by Imler et al.; PCT/FR94/00851 (WO 95/02697) by Perrocaudet et al., PCT/US95/14793 (WO96/14061) by Wang et al.

AA V Vectors

Yet another viral vector system useful for delivery of the subject polynucleotides is the adeno-associated virus (AAV). Adeno-associated virus is a naturally occurring defective virus that requires another virus, such as an adenovirus or a herpes virus, as a helper virus for efficient replication and a productive life cycle. (For a review, see Muzyczka et al., Curr. Topics in Micro. and Immunol. (1992) 158:97-129).

AAV has not been associated with the cause of any disease. AAV is not a transforming or oncogenic virus. AAV integration into chromosomes of human cell lines does not cause any significant alteration in the growth properties or morphological characteristics of the cells.

AAV is also one of the few viruses that may integrate its DNA into non-dividing cells, e.g., pulmonary epithelial cells, and exhibits, a high frequency of stable integration (see for example Flotte et al., (1992) Am. J. Respir. Cell. Mol. Biol. 7:349-356; Samulski et al., (1989) J. Virol. 63:3822-3828; and McLaughlin et al., (1989) J. Virol. 62:1963-1973). Vectors containing as little as 300 base pairs of AAV can be packaged and can integrate. Space for exogenous DNA is limited to about 4.5 kb. An AAV vector such as that described in Tratschin et al., (1985) Mol. Cell. Biol. 5:3251-3260 can be used to introduce DNA into cells. A variety of nucleic acids have been introduced into different cell types using AAV vectors (see for example Hermonat et al., (1984) PNAS USA 81:6466-6470; Tratschin et al., (1985) Mol. Cell. Biol. 4:2072-2081; Wondisford et al., (1988) Mol. Endocrinol. 2:32-39; Tratschin et al., (1984) J. Virol. 51:611-619; and Flotte et al., (1993) J. Biol. Chem. 268:3781-3790).

The AAV-based expression vector to be used typically includes the 145 nucleotide AAV inverted terminal repeats (ITRs) flanking a restriction site that can be used for subcloning of the transgene, either directly using the restriction site available, or by excision of the transgene with restriction enzymes followed by blunting of the ends, ligation of appropriate DNA linkers, restriction digestion, and ligation into the site between the ITRs. The capacity of AAV vectors is usually about 4.4 kb (Kotin, R. M., Human Gene Therapy 5:793-801, 1994 and Flotte, et al. J. Biol. Chem. 268:3781-3790, 1993).

AAV stocks can be produced as described in Hermonat and Muzyczka (1984) PNAS 81:6466, modified by using the pAAV/Ad described by Samulski et al. (1989) J. Virol. 63:3822. Concentration and purification of the virus can be achieved by reported methods such as banding in cesium chloride gradients, as was used for the initial report of AAV vector expression in vivo (Flotte, et al. J. Biol. Chem. 268:3781-3790, 1993) or chromatographic purification, as described in O'Riordan et al., WO97/08298. Methods for in vitro packaging AAV vectors are also available and have the advantage that there is no size limitation of the DNA packaged into the particles (see, U.S. Pat. No. 5,688,676, by Zhou et al., issued Nov. 18, 1997). This procedure involves the preparation of cell free packaging extracts.

### Hybrid Adenovirus-AA V Vectors

Hybrid Adenovirus-AAV vectors have been generated and are typically represented by an adenovirus capsid containing a nucleic acid comprising a portion of an adenovirus, and 5′ and 3′ inverted terminal repeat sequences from an AAV which flank a selected transgene under the control of a promoter. See e.g. Wilson et al, International Patent Application Publication No. WO 96/13598. This hybrid vector is characterized by high titer transgene delivery to a host cell and the ability to stably integrate the transgene into the host cell chromosome in the presence of the rep gene. This virus is capable of infecting virtually all cell types (conferred by its adenovirus sequences) and stable long term transgene integration into the host cell genome (conferred by its AAV sequences).

The adenovirus nucleic acid sequences employed in this vector can range from a minimum sequence amount, which requires the use of a helper virus to produce the hybrid virus particle, to only selected deletions of adenovirus genes, which deleted gene products can be supplied in the hybrid viral process by a packaging cell. For example, a hybrid virus can comprise the 5′ and 3′ inverted terminal repeat (ITR) sequences of an adenovirus (which function as origins of replication). The left terminal sequence (5′) sequence of the Ad5 genome that can be used spans bp 1 to about 360 of the conventional adenovirus genome (also referred to as map units 0-1) and includes the 5′ ITR and the packaging/enhancer domain. The 3′ adenovirus sequences of the hybrid virus include the right terminal 3′ ITR sequence which is about 580 nucleotides (about bp 35,353-end of the adenovirus, referred to as about map units 98.4-100).

For additional detailed guidance on adenovirus and hybrid adenovirus-AAV technology which may be useful in the practice of the subject invention, including methods and materials for the incorporation of a transgene, the propagation and purification of recombinant virus containing the transgene, and its use in transfecting cells and mammals, see also Wilson et al, WO 94/28938, WO 96/13597 and WO 96/26285, and references cited therein.

### Retroviruses

In order to construct a retroviral vector, a nucleic acid of interest is inserted into the viral genome in the place of certain viral sequences to produce a virus that is replication-defective. In order to produce virions, a packaging cell line containing the gag, pol, and env genes but without the LTR and psi components is constructed (Mann et al. (1983) Cell 33:153). When a recombinant plasmid containing a human cDNA, together with the retroviral LTR and psi sequences is introduced into this cell line (by calcium phosphate precipitation for example), the psi sequence allows the RNA transcript of the recombinant plasmid to be packaged into viral particles, which are then secreted into the culture media (Nicolas and Rubenstein (1988) “Retroviral Vectors”, In: Rodriguez and Denhardt ed. Vectors: A Survey of Molecular Cloning Vectors and their Uses. Stoneham:Butterworth; Temin, (1986) “Retrovirus Vectors for Gene Transfer: Efficient Integration into and Expression of Exogenous DNA in Vertebrate Cell Genome”, In: Kucherlapati ed. Gene Transfer. New York: Plenum Press; Mann et al., 1983, supra). The media containing the recombinant retroviruses is then collected, optionally concentrated, and used for gene transfer. Retroviral vectors are able to infect a broad variety of cell types. Integration and stable expression require the division of host cells (Paskind et al. (1975) Virology 67:242). This aspect is particularly relevant for the treatnent of PVR, since these vectors allow selective targeting of cells which proliferate, i.e., selective targeting of the cells in the epiretinal membrane, since these are the only ones proliferating in eyes of PVR subjects.

A major prerequisite for the use of retroviruses is to ensure the safety of their use, particularly with regard to the possibility of the spread of wild-type virus in the cell population. The development of specialized cell lines (termed “packaging cells”) which produce only replication-defective retroviruses has increased the utility of retroviruses for gene therapy, and defective retroviruses are well characterized for use in gene transfer for gene therapy purposes (for a review see Miller, A. D. (1990) Blood 76:271). Thus, recombinant retrovirus can be constructed in which part of the retroviral coding sequence (gag, pol, env) has been replaced by nucleic acid encoding a protein of the present invention, e.g., a transcriptional activator, rendering the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al., (eds.) Greene Publishing Associates, (1989), Sections 9.10-9.14 and other standard laboratory manuals. Examples of suitable retroviruses include pLJ, pZIP, pWE and pEM which are well known to those skilled in the art. A preferred retroviral vector is a pSR MSVtkNeo (Muller et al. (1991) Mol. Cell. Biol. 11:1785 and pSR MSV(XbaI) (Sawyers et al. (1995) J. Exp. Med. 181:307) and derivatives thereof. For example, the unique BamHI sites in both of these vectors can be removed by digesting the vectors with BamHI, filling in with Klenow and religating to produce pSMTN2 and pSMTX2, respectively, as described in PCT/US96/09948 by Clackson et al. Examples of suitable packaging virus lines for preparing both ecotropic and amphotropic retroviral systems include Crip, Cre, 2 and Am.

Retroviruses, including lentiviruses, have been used to introduce a variety of genes into many different cell types, including neural cells, epithelial cells, retinal cells, endothelial cells, lymphocytes, myoblasts, hepatocytes, bone marrow cells, in vitro and/or in vivo (see for example, review by Federico (1999) Curr. Opin. Biotechnol. 10:448; Eglitis et al., (1985) Science 230:1395-1398; Danos and Mulligan, (1988) PNAS USA 85:6460-6464; Wilson et al., (1988) PNAS USA 85:3014-3018; Armentano et al., (1990) PNAS USA 87:6141-6145; Huber et al., (1991) PNAS USA 88:8039-8043; Ferry et al., (1991) PNAS USA 88:8377-8381; Chowdhury et al., (1991) Science 254:1802-1805; van Beusechem et al., (1992) PNAS USA 89:7640-7644; Kay et al., (1992) Human Gene Therapy 3:641-647; Dai et al., (1992) PNAS USA 89:10892-10895; Hwu et al., (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573).

Furthermore, it has been shown that it is possible to limit the infection spectrum of retroviruses and consequently of retroviral-based vectors, by modifying the viral packaging proteins on the surface of the viral particle (see, for example PCT publications WO93/25234, WO94/06920, and WO94/11524). For instance, strategies for the modification of the infection spectrum of retroviral vectors include: coupling antibodies specific for cell surface antigens to the viral env protein (Roux et al., (1989) PNAS USA 86:9079-9083; Julan et al., (1992) J. Gen Virol 73:3251-3255; and Goud et al., (1983) Virology 163:251-254); or coupling cell surface ligands to the viral env proteins (Neda et al., (1991) J. Biol. Chem. 266:14143-14146). Coupling can be in the form of the chemical cross-linking with a protein or other variety (e.g. lactose to convert the env protein to an asialoglycoprotein), as well as by generating fusion proteins (e.g. single-chain antibody/env fusion proteins). This technique, while useful to limit or otherwise direct the infection to certain tissue types, and can also be used to convert an ecotropic vector in to an amphotropic vector.

### Other Viral Systems

Other viral vector systems that can be used to deliver a polynucleotide of the invention have been derived from vaccinia virus, alphavirus, poxvirus, arena virus, polio virus, and the like. Such vectors offer several attractive features for various mammalian cells. (Ridgeway (1988) In: Rodriguez R L, Denhardt D T, ed. Vectors: A survey of molecular cloning vectors and their uses. Stoneham: Butterworth; Baichwal and Sugden (1986) In: Kucherlapati R, ed. Gene transfer. New York: Plenum Press; Coupar et al. (1988) Gene, 68:1-10; Walther and Stein (2000) Drugs 60:249-71; Timiryasova et al. (2001) J Gene Med 3:468-77; Schlesinger (2001) Expert Opin Biol Ther 1:177-91; Khromykh (2000) Curr Opin Mol Ther 2:555-69; Friedmann (1989) Science, 244:1275-1281; Ridgeway, 1988, supra; Baichwal and Sugden, 1986, supra; Coupar et al., 1988; Horwich et al. (1990) J. Virol., 64:642-650).

### EXEMPLIFICATION

### Example 1

### Metal Binding by Metallothionein

Gold(I) binding to metallothionein was assessed with both electrospray ionization (ESI) mass spectrometry and matrix assisted laser de-absorption ionization (MALDI) mass spectrometry. Specifically, two metal binding states were examined. The first is the lower gold-metallothionein state with up to about 17 gold atoms bound. The second state is a high gold-metallothionein state containing as many as 40 gold atoms. These results suggest a novel gold binding scheme that may be more akin to commercial gold cluster formation.

ESI mass spectrometry has the advantage that it can be used to record masses of biological complexes with high resolution. Unlike other forms of mass spectrometry, ESI can be performed under soft, non-denaturing conditions. Hence, ionic interactions, such as those of metal atoms bound by proteins, can be detected. Within the ESI mass spectrometer, charged protein complexes are electrostatically accelerated into a time of flight tube with a detector at the end of the flight path. The higher the charge and smaller the mass, the faster the charged molecules passes through to the detector. Thus, the timing of events recorded at the detector corresponds to a mass to charge ratio. Soft ionization is achieved by aerosolizing the sample through a nozzle held at a high voltage and by subsequently dehydrating the nebulized droplets to obtained charged protein complexes. Recorded mass to charge ratio spectra can be deconvoluted into precise masses since peaks resulting from different charged states of the same protein can only maximally sum at the least common multiple of the charged states. The collected spectra yield detailed information about stoichiometries, information about relative amounts of various species, and, with adjustment of conditions, information about complex stability. This method has been used to examine metallothioneins from several species with zinc, cadmium, or copper bound. Furthermore, ESI mass spectra of metal titrated apo-metallothionein show complexes in partially filled states and degrees of metal binding cooperativity [61]. To date, however, no gold-metallothionein complexes have been examined with this method.

Unlike ESI mass spectrometry, MALDI mass spectrometry has not been used in the study of metallothionein. However, MALDI mass spectrometry has been extensively used to analyze gold clusters [22, 23, 62]. This technique has the advantage that it requires less material than ESI mass spectrometry, and it has an extended mass to charge ratio range up to several hundred kilodaltons. However, it is generally considered to be more denaturing and as such does not detect weak interactions. Although this would make MALDI mass spectrometry unsuitable for more weakly bound zinc-metallothionein complexes, gold-metallothionein complexes, which share similar bond strengths as those within gold nanoclusters, should be detectable with this technique.

### Materials and Methods

### Sample Preparation

Protein for mass spectrometry experiments was prepared from Zinc-7-metallothionein (M954) that was obtained lyophilized from Sigma Chemical Corporation. Specifically, 5 mg of protein was re-hydrated in 0.5 mL of 25 mM Tris-HCl pH 7.5 and then flash frozen with liquid nitrogen in 50 uL aliquots. On the day of an experiment, enough aliquots to prepare samples were defrosted and stored on ice until their time of use.

### Sample Incubation

Individual samples were prepared for mass spectrometry by diluting the defrosted protein stock solution to a final protein concentration of 1 mg/mL with final sample volumes of 100 uL. For all samples, 25 mM Tris-HCl was used as buffer. Samples were prepared by first mixing all non-protein sample components together followed by adding 10 uL of the protein stock solution. Typically, non-protein components were prepared as 10× stocks for addition to samples. Once prepared, samples were incubated at 37 degrees Celsius for 3 hours before desalting.

### Mass Spectrometry

For ESI mass spectrometry, samples were desalted by buffer exchange in a spin concentration device. First, the sample was concentrated to 50 uL by spinning the sample within a YM-3 microcentricon (Amicon) at 12000×G in a tabletop microfuge. Desalting was performed by repeated dilution to 500 uL in 25 mM Tris-HCl followed by re-concentration to 50 uL in the YM-3 microcentricon for two times. This was repeated 3 more times using a suitable ESI mass spectrometry buffer. For these experiments, the buffer was a 5 mM ammonium acetate buffer pH 6.0. Finally, the sample was diluted to 100 uL with 5 mM ammonium acetate buffer pH 6.0 with 40% methanol to give a final concentration of 20% methanol in the sample. For an apo-metallothionein control, a small volume (less than 5 uL) of 0.1 M acetic acid was added to a Zinc-7-metallothionein sample to lower the pH below pH 4 and cause release of zinc from the protein. All samples were spun through a 0.4 um spin filter (Amicon).

All ESI mass spectrometry samples were run at the Brandeis University Biochemistry Core Facility on a Perseptive Biosystems Mariner. Samples were typically diluted an additional 5 or 20 fold at the time of recording in a 5 mM ammonium acetate buffer pH 6.0 with 20% methanol to obtain a strong signal with minimal protein amounts. Samples were injected at a rate of 3-5 uL/min and collected over a mass to charge range of 500 to 4000. Nozzle potential and detector potential were adjusted to obtain strong clean spectra. All samples were collected in positive ion mode. The instrument calibration was verified on the day of the experiment using apo-myoglobin (A8673, Sigma Chemical Corporation) in a 5 mM ammonium acetate buffer pH 6.0 with 20% methanol.

MALDI mass spectrometry samples were prepared and incubated as above. Similar to ESI mass spectrometry samples, MALDI samples were also desalted. This was accomplished by concentrating the sample to 50 uL with a YM-3 microcentricon in a tabletop microfuge. Likewise, this was followed by repeated dilution to 500 uL and subsequent re-concentration to 50 uL in the YM-3 microcentricon for three times. However, for these experiments, a 10 mM Tris-HCl buffer pH 7.5 was used.

All MALDI mass spectrometry samples were run at the Brandeis University Biochemistry Core Facility on a Perseptive Biosystems Voyager (Framingham, Mass.). On the day of the experiment, fresh matrix solution was made. One of two matrixes was used for experiments. Either a 5 mg/mL sinapinic acid (SA) (D7927, Sigma Chemical Corporation) solution in a water:acetonitrile (50%:50%) or a 10 mg/mL 6-azo-thiothymadine (ATT) (27, 551-4, Aldrich Chenical Company) in a water:acetonitrile (50%:50%) were used as matrixes. Samples were diluted 10 or 20 fold in matrix solution to obtain a strong signal with minimal protein amounts. Samples diluted in matrixes were then spotted onto the sample plate with 2 uL per well. Droplets were allowed to dry and then placed into the spectrometer. Spectra were collected with a 25,000 V acceleration voltage in positive ion mode over a range of 3000 to 100,000 mass/charge ratio.

### Transmission Electronic Microscopy

Desalted samples of metallothionein incubated with 20 molar equivalents of aurothiomalate within ammonium acetate buffer were saved for viewing within the transmission electron microscope. Quantifoil (Jena, Germany) grids with 1 micron diameter holes were used to support thin (<200 Angstrom) continuous carbon foils. Carbon foils were prepared by depositing carbon onto freshly cleaved smooth mica in an Edwards vacuum evaporator. These foils were then floated on water, and pieces of the foil were picked up on to the Quantifoil grids. The grids were set a side to dry for at least 24 hours before proceeding. Grids were negative glow discharged in air and 3 uL of sample was applied to the thin carbon surface side of the grid. After 30 second the grids were rinsed twice with ammonium acetate buffer. Excess buffer was carefully blotted with Whatman filter paper from the edge of grids so as to not touch the viewing area. The grids were allowed to fully dry before placing in the transmission electron microscope.

### Results

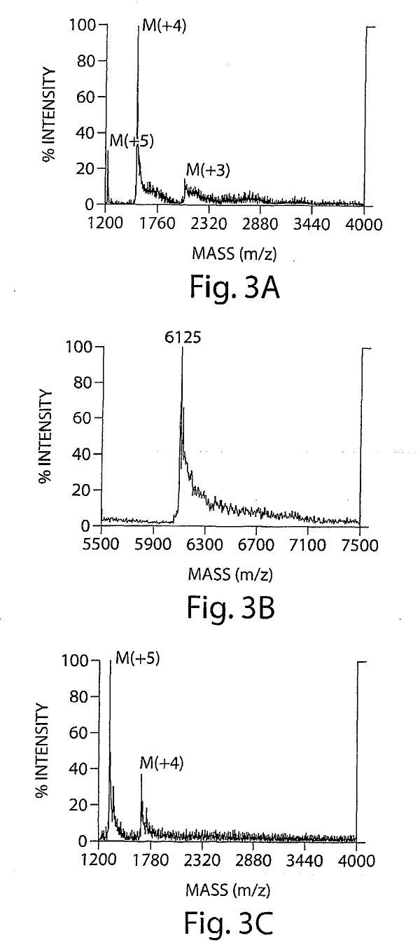

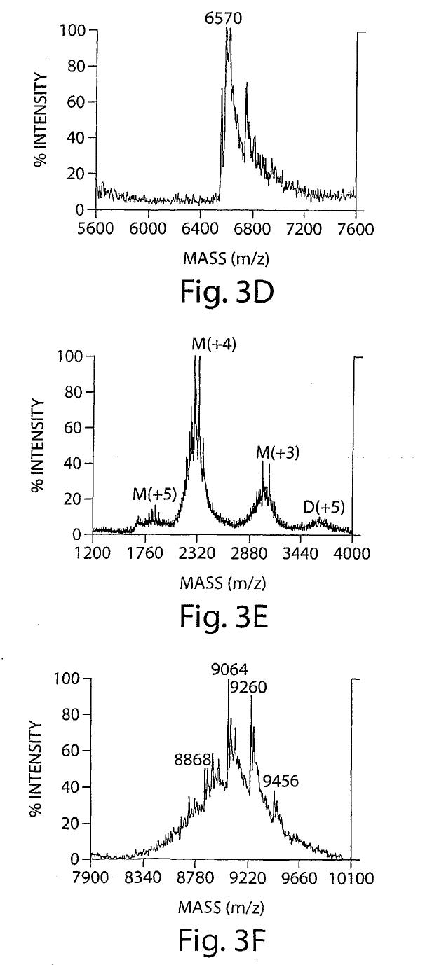

FIG. 3 shows a comparison of ESI-mass spectrometry results for apo-metallothionein, Zn-metallothionein, and Au-metallothionein. Panels A, C, and D show typical collected mass spectra recorded as a mass to charge ratio for the three types of complexes, respectively. All collected spectra show multiple charged peaks corresponding to the +5, +4, and +3 peaks resulting from monomers as seen in individual panels counting peaks from left to right. Occasionally, dimer peaks corresponding to the +9, +7, and +5 were weakly observed, noting the +10, +8, and +6 peaks are obscured within the stronger monomer signals. An example of a dimer peak can be seen as in the +5 dimer Au-metallothionein peak seen in panel E. Zero charge mass deconvoluted results of the spectra in panels A, C, and E are shown in panes B, D, and F, respectively. Deconvolution averages the multiple charge peaks within each recorded spectra to increase signal to noise. Furthermore, confidence in the assigned charge values for individual spectral peaks can be assured since reinforcement of signal within the deconvoluted spectra is achieved only upon providing appropriate charge values.

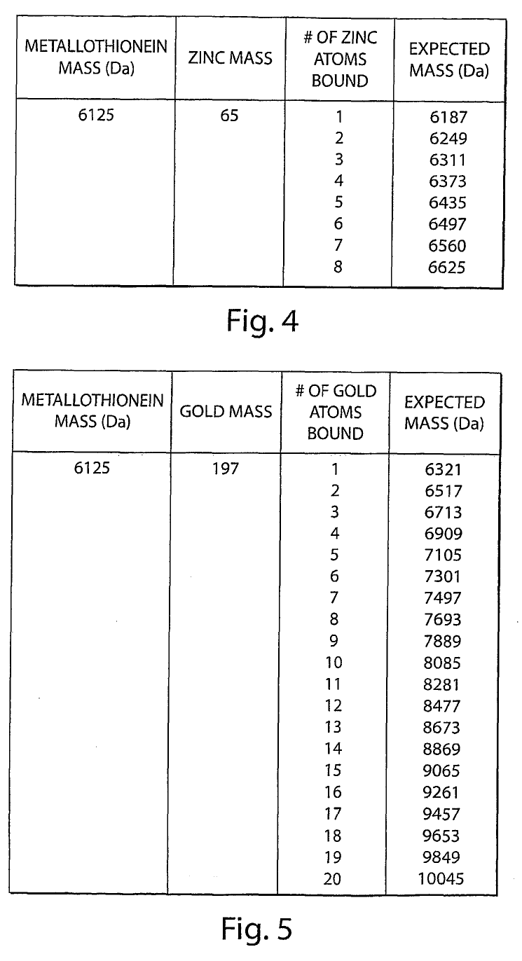

Comparison of the collected spectra show increased mass due to metal binding. Lowering the pH of a Zn-7-metallothionein containing sample created the apo-metallothionein shown in panel A. The weak extended shoulders of the +4 and +3 peaks most likely resulted from incomplete release of zinc from the protein during the removal process. The zero charge deconvoluted signal of panel B contains a sharp peak at a mass of 6125 amu that corresponds perfectly to the expected and previously reported value for rabbit liver metallothionein II [63]. Panels C and D, showing results for Zn-metallothionein, contain noticeable shifts of peaks associated with the increased mass from the apo-metallothionein. Panel D, the zero charge mass spectrum, shows a broader distribution composed of more than one peak. The main peak has a mass of 6570 amu that corresponds to 7 zinc atoms bound to the protein (see Table 1 in FIG. 4). Similar varied distributions have been witnessed in previous ESI mass spectrometry results for metal bound metallothioneins [61]. The extended shoulder witnessed in Panel D may be suggestive of additional bound atoms of zinc and other elements to some of the complex. Also, the strong peak at about 6770 amu is most likely a bound gold atom, a contaminant of a previously analyzed ESI sample. These results give confidence that this method can produce meaningful results for metal bound metallothioneins.

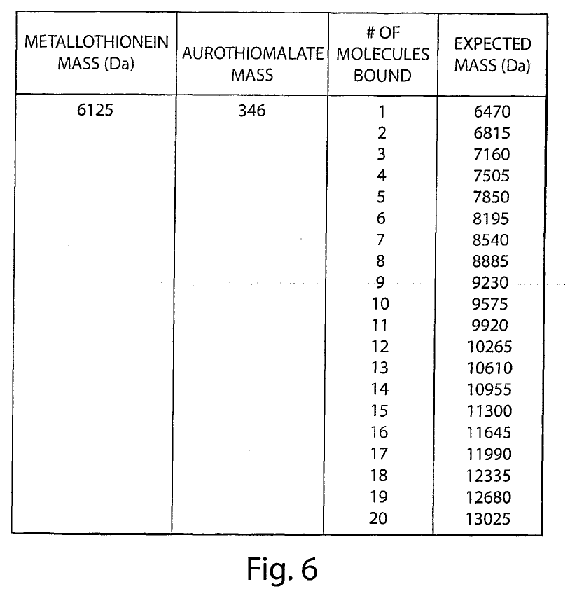

Most interesting is the extremely large shifts associated with Zn-7-metallothionein incubated with 20 molar equivalents of the anti-arthritic drug, aurothiomalate. Previous studies have shown that this drug completely removes zinc from metallothionein under these conditions [53]. The collected spectrum and zero charge spectrum shown in panels E and F, respectively, show much larger mass to charge ratio shifts and wider peak distributions than the zinc bound metallothionein samples. The zero charge state results show a striking periodicity of around a 196 atomic mass units peak-to-peak. This is strongly suggestive of the addition of individual gold atoms without the presence of carrier ligand. Table 2 (FIG. 5) and Table 3 (FIG. 6) tabulate the expected values for peaks arising from gold additions as single atoms and as complete aurothiomalate molecules, respectively. The series of periodic peak at 8868 amu, 9064 amu, 9260 amu, and 9456 amu almost perfectly match the expected values for the 14, 15, 16, and 17 gold atom peaks in Table 2 (FIG. 5). This strong agreement between the observed and expected results gives great confidence that the assumed mode of binding as single gold atoms is correct.

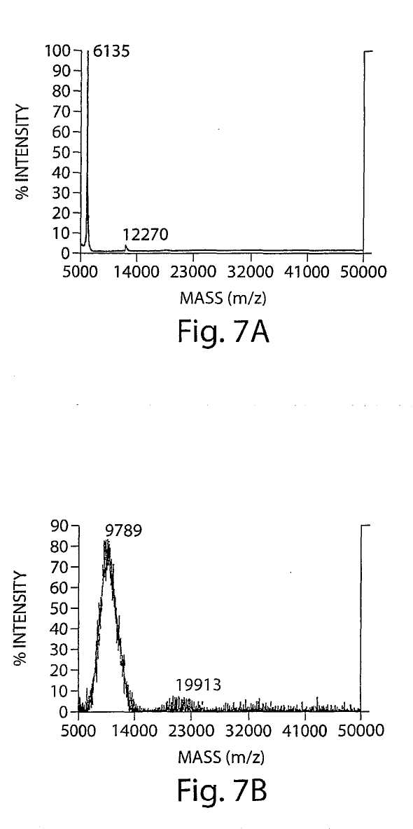

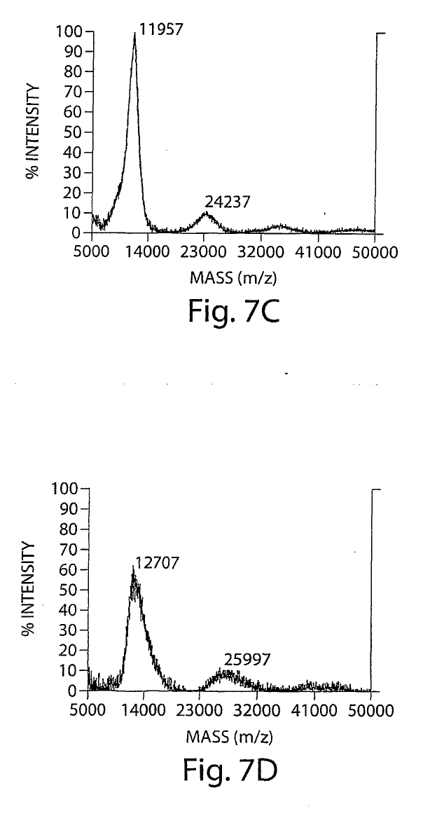

Since the strength of gold thiolate bonds is expected to be strong and is able to withstand pH below pH 2, resolving metallothionein gold complexes with MALDI mass spectrometry was attempted. This is the standard method used to resolve masses of commercial gold nanoclusters [22, 64]. FIG. 7 displays a series of MALDI mass spectrometry results of Zn-7-metallothionein incubated with various concentrations of aurothiomalate. Panel A shows a typical control sample of the protein without incubation with aurothiomalate. The mass observed of 6135 can be attributed to the apo-metallothionein state. This metal loss is expected given the matrix is about pH 2. Thus, zinc, which is removed below pH 5, is no longer associated with protein. Also noticeable is the presences of dimer and trimer states of the protein. Panel B shows the typical result of metallothionein incubated with 20 molar equivalents of the gold containing compound. Like the ESI-mass spectrometry results shown in FIG. 3 Panels E and F, a large mass shift and wider distribution are observed for aurothiomalate-incubated metallothioneins. Hence, gold remains bound to the protein during the MALDI sample preparation and ionization/deabsorption process. This technique proved useful since obtaining ESI mass spectrometry results became more difficult when large molar excesses of aurothiomalate were used.

The ESI and MALDI mass spectrometry results appear slightly different. Clearly, the collected ESI mass spectra are better resolved. Also, the MALDI results appear to have a wider distribution as seen in FIG. 7 panel B where the monomer ranges from about 6000 amu to about 13,000 amu. These qualities may be associated with the ionization/deabsorption process. A similar result has been observed when comparing ESI and MALDI results from gold nanoclusters. The ionization/deabsorption process is believed to cause some degree of particle fragmentation. Thus, observed peaks may result from complexes initially containing more mass, and small amounts of fragmentation should limit peak-to-peak resolution [22]. However, MALDI has the advantage that it is consistently easier to use, provides strong signal, and allows for detection of larger masses at lower charged states.

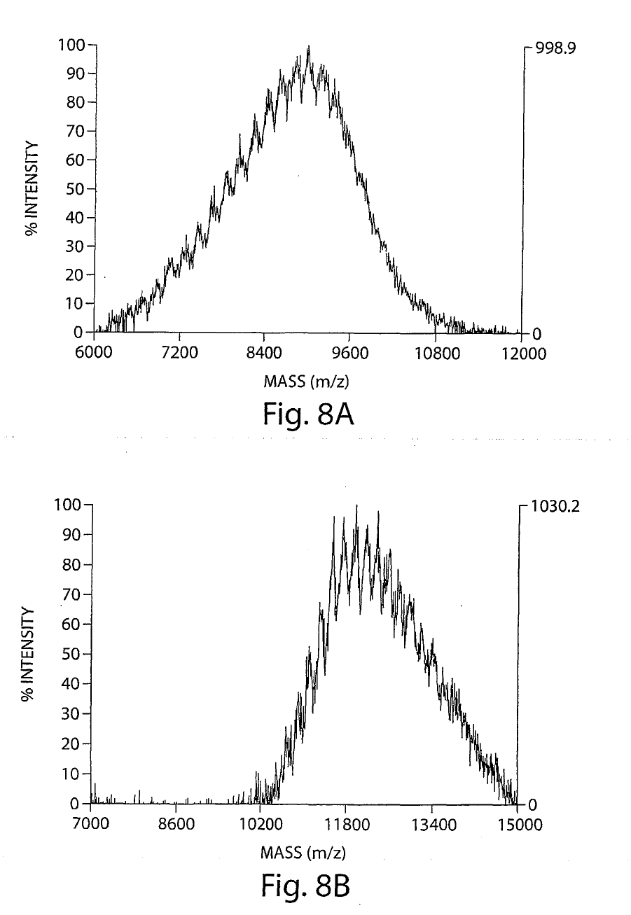

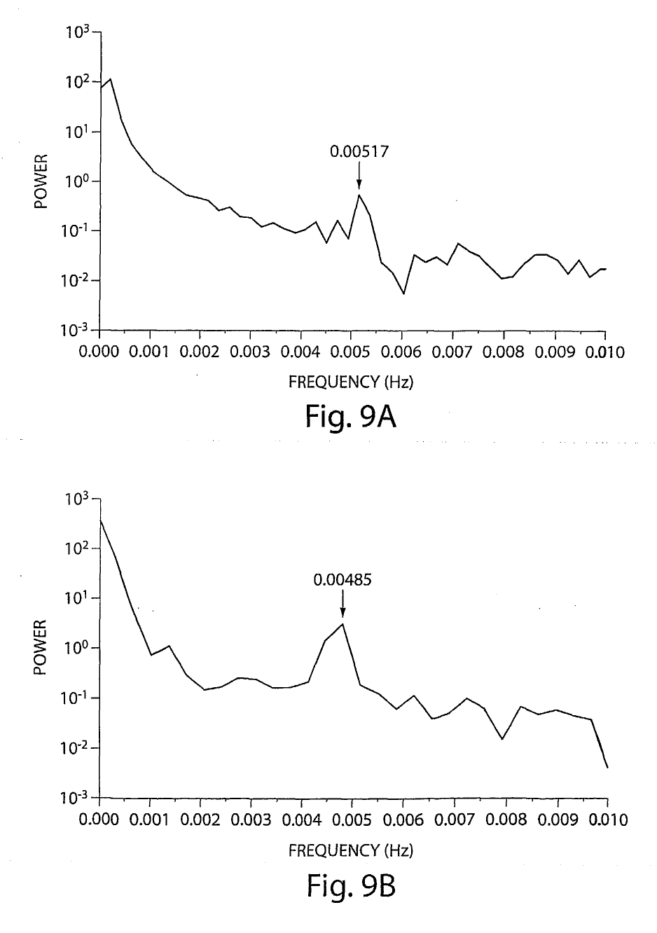

Although MALDI mass spectra often compared well to the result in FIG. 7 Panel B, on several occasions a distinctly different result was observed. The extent of gold binding in the presence of 200 molar equivalents of aurothiomalate can be seen in FIG. 7 Panels C and D. These spectra show extremely shifted mass peaks. Assuming the gold binding is similar to that suggested in the ESI mass spectrometry results, the peaks witnessed in Panels C and D are attributable to the binding of 30 and 34 gold atoms to a single metallothionein, respectively. This would imply a gold to cysteine ratio of greater than 1:1. Confidence that these highly gold-reacted metallothionein complexes share an equivalent mode of gold binding to the typically observed gold-metallothionein results can be gained from the occasional periodicity witnessed in some MALDI mass spectra. FIG. 8 shows spectra of both low (≦20 bound atoms) and high (>20 bound atoms) gold metallothionein forms. Both spectra show a strong 180 to 200 atomic mass unit periodicity. The persistence of this periodicity within spectra observed for both the low and high gold-bound metallothionein forms allows for almost a direct counting of gold atoms from the series of peaks. To evaluate the periodicity, Fourier transforms of the mass spectrometry signals were computed. These are shown in FIG. 9. The peaks at 0.00517 Hertz and 0.00485 Hertz correspond to a periodicity of 193.4 amu and 206.2 amu in the mass spectrometry spectra, respectively. These values, like the previous ESI mass spectrometry results, suggest gold binding occurs by the addition of single atoms not whole aurothiomalate molecules. The likely reasons that this periodicity is not observed in all MALDI results may be the degree of spectral smoothing during data collection. This attenuation of peak resolution may result from a lower degree of sample desalting during preparation or possibly occurs as a result of the previously reported ionization-induced fragmentation.

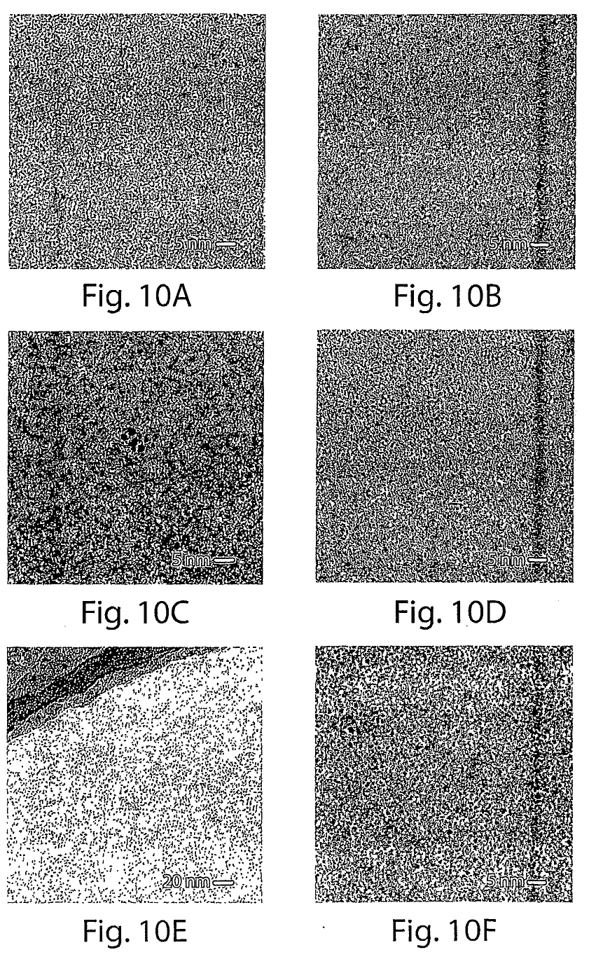

Since mass spectrometry results hinted at gold contents between those known for the Undecagold® and Nanogold® TEM labels, TEM images of gold-bound metallothionein samples were taken. The protein was placed on a thin carbon foil (<200 Angstroms thick) suspended over a holey Quantifoil® grid to provide as low a background as possible. No stain was used on these samples. Images of metallothionein samples containing 15 to 17 bound gold atoms are shown at two different magnifications in FIG. 10 Panels E and F. Small dense particles ranging from <1 nm to about 4 nm can be seen distributed over the carbon foil surface. Since these samples are prepared within volatile ammonium acetate buffer, these dense particles are most likely gold metallothionein and not salt. As controls, images from grids with buffer and aurothiomalate (Panels A and C), buffer alone (Panel B) and buffer with Zn₇-metallothionein (Panels D) were recorded. The only significant densities seen in these controls are the occasional large aggregates witnessed in the aurothiomalate control (Panel C). These aggregates are most likely partially undissolved aurothiomalate which is unable to pass through the 3 kDa cutoff of the filter unit used for buffer exchange. Small metallothionein-like clusters were not observed on multiple grids. Absorbance readings of the aurothiomalate sample at 280 nm showed a 30-fold decrease in the total aurothiomalate from the initial sample as a result of buffer exchange.

The different sizes observed within the metallothionein sample (Panels E and F) may be attributed to the oligomerization observed in the mass spectrometry results. Within MALDI mass spectra, oligomers as large as tetramers were observed. However, MALDI detection becomes more difficult as the mass increases. Therefore, it is possible larger oligomers were not detected. Nevertheless, the appearance of these clusters is strikingly similar to other gold nanoclusters.

### Example 2

### Visualization of MBP-Metallothionein Fusion Proteins

A commercially available maltose binding protein (MBP) purification system was used to achieve visualization of known metallothionein complexes. The use of a purification tag had the advantage that it could be cloned with one or more concatenated metallothionein sequences so that each of the different constructs could be produced, purified, evaluated for gold bind ability, and visualized.

In addition to using the TEM for visualizing metallothionein gold complexes, the scanning transmission electron microscope (STEM) was also used. STEM, by virtue of its method of recording images, has several advantages over TEM when examining samples containing metal atoms. In the TEM, an electron beam is transmitted through a relatively large region of sample (typically 100 nm or more in diameter), and scattered electrons are refocused with lenses to create an image using a similar lens setup to a conventional light microscope. In the STEM, however, the electron beam is focused into a diameter as narrow as 1.25 Å and slowly moved across a region of sample. Unlike TEM, scattered electrons are not refocused in to an image. Instead, a series of annular detectors collects particular angularly scattered electrons at each point within a sample. The signal collected from each of the annular detectors from the known positions in the sample can be used to create a series of images corresponding to different angles of scattering. The advantage of multiple detectors is that the combined information can be used to calculate the masses of particles in the image as well as an average density of the material at each pixel. Also, the annular detectors can be placed such that the majority of electrons detected are from elastically scattered electrons. The combination of the narrow beam and the collection of mainly elastically scattered electron gives STEM a high signal to noise ratio, which can allow visualization of proteins as small as 50,000 Daltons.

Importantly, STEM is able to visualize samples containing metal atoms. Since atoms with large atomic numbers, such as gold, have larger scattering factors as well as have a greater propensity to scatter electrons elastically than atoms with small atomic number, such as those in biological samples, larger atomic number atoms are distinctly visible in STEM images. Although experimental measurements of these values have been made, it is important to note that these depend on the acceleration voltage used. As approximate values for these factors at conditions close to those used to collect the STEM data presented here, the following experimental values have been reported: For electrons accelerated at 65 keV, the scattering factor of gold is about 8, and the scattering factor of carbon is 0.76 [69]. In addition, the ratio of elastic to inelastic events reported for electrons accelerated at 85 keV is about 3 to 1 for gold, while it is reversed for carbon at a ratio of about 1 to 3 [70]. Hence gold should be much more detectible than biological materials.

### Materials and Methods

Cloning of MBP Fusion Proteins with Single and Multiple Metallothionein Copies

The MBP fusion cloning vector pMal-c2x, which expresses MBP with a linker region containing a Factor Xa site, was purchased from New England Biolabs (Beverly, Mass.). Metallothionein with the gene cloned within a pET3-d vector (Novagen, Madison, Wis.) between the NcoI and BamHI sites and was designated pET3-dMT.

The first fusion produced contained metallothionein directionally cloned onto the C-terminus of MBP and was designated pMal-c2x-MT. For this construct, the metallothionein gene was produced by PCR from pET3-dMT. For the PCR reactions, the primer (5′-CTCGGGATCGAGGGAAGGATTTCAAGATATACCATGGACCCC-3′) (SEQ ID NO: 2), which codes for the MBP linker region fused in frame to metallothionein gene and contains an XmnI site and NcoI site, and the primer (5′-GACTCTAGAGGATCCTAGGCACAGCACGTG-3′) (SEQ ID NO: 3), which contains the metallothionein gene stop codon and a subsequent BamHI site, were used. Directional cloning was performed using XmnI and BamHI from both the PCR product and pMal-c2x vector.

The second fusion was produced containing two copies of the metallothionein gene fused in frame and was designated pMal-c2x-MT2. In this new construct, the only translational modification was an alanine changed to an aspartic acid in the coding junction between the two metallothionein genes. The second copy of the metallothionein gene was added to pMal-c2x-MT plasmid by directional cloning. For this, the second metallothionein gene was produced by PCR from pET3-dMT using primer (5′-CTCGGGATCGAGGGAAGGATTTCAAGATATACCA TGGACCCC-3′) (SEQ ID NO: 2), which codes for the MBP linker region fused in frame to metallothionein gene and contains an XmnI site and NcoI site, and primer (5′-GTGACCACATGTCACAGCACGTGCACTTGTCC-3′) (SEQ ID NO: 4), which contains an AflIII site at the metallothionein-metallothionein junction. The pMal-c2x-MT plasmid was prepared by digesting with XmnI and NcoI, and the PCR product was prepared with XmnI and AflIII. Since NcoI and AflIII produce equivalent overhangs on the DNA ends, the second copy of metallothionein can be inserted with removal of both restriction sites at this ligation junction. This is extremely useful since it leaves a unique NcoI site that can be used to add future additional metallothionein genes using the same PCR product.

Since all restriction sites in the newly formed construct are the same as their parent plasmids, and XmnI produces blunt ends for cloning, properly fused constructs were isolated by first screening for over-expressed proteins of the correct expected molecular weight. This was accomplished by transforming ligation reactions into the NovaBlue (Novagen) E. coli bacterial strain. Then, isolated colonies grown in LB with selection were checked for expression after induction with 1 mM isopropyl-β-D-thiogalactopyranoside (IPTG) for 2 hours. Clarified cell extracts from individual colonies were run on SDS polyacrylamide gels to check for the molecular weight of the induced protein. Next, colonies showing over-expressed protein of the expected molecular weight were grown a second time followed by isolation of the newly formed plasmid. The DNA was checked by restriction analysis and was subsequently sequenced.

### Expression of MBP Fusion Proteins

Sequence verified plasmids were transformed into the bacterial strain TB1 of E. coli. Starter cultures in LB were begun from single colonies and grown with selection overnight at 30 degrees Celsius with aeration. The next morning 1 L growth cultures inoculated with 5 mL of overnight culture were grown with selection and 1 percent glucose until an OD₆₀₀ of 0.5 was reached. At this point, protein production was induced with 0.2 mM IPTG. After 0.5 hours zinc sulfate was added to 0.2 mM in the hope of filling metal sites within the metallothionein portions of proteins. Cultures were grown an additional 1.5 hours. After 2 hours of induction, cells were pelleted at 6000×g. Cell pellets were placed in 50 mL conical tubes, flash frozen in liquid nitrogen, and stored at −70 degrees Celsius until the day of purification.

### Purification of MBP Fusion Proteins

Fusion proteins of MBP with metallothionein were purified with slight modification to the New England Biolabs standard MBP purification procedure. Briefly, cells were defrosted the day of purification and all steps of the procedure were performed at 4 degrees Celsius. Cells were resuspended with the addition 25 mL of Wash buffer (20 mM Trizma Base pH 7.5, 150 mM sodium chloride, and 0.1 mM 2-mercaptoethanol) (all from Sigma Chemical Corp, St. Louis, Mo.). Suspensions were sonicated 8 times with 30 second pulses and 1 minute rest periods on ice with a Branson 2000 sonicator to lyse cells. Lysis was monitored with Biorad Total Protein Concentration (Hercules, Calif.) solution.

Once lysed, cell suspensions were spun at 9000×g in an Eppendorf 5804R Centrifuge. The resulting supernatant was diluted in Wash Buffer to 100 mL and mixed. The diluted supernatant was then load by gravity over a pre-equilibrated 5 mL amylose column (New England Biolabs). After all of the supernatant was loaded, the column was washed with 10 column volumes of Wash buffer. Bound protein was eluted in 0.5 mL fractions in Wash buffer supplemented with 10 mM maltose (Sigma Chemical Corp) but without 2-mercaptoethanol or other reducing agents. Protein concentrations of eluted fractions were checked with Biorad Total Protein Concentration solution. Typically, eluted fractions were found to have about 5 mg/mL concentrations in their peak fractions and aliquots were flash frozen in liquid nitrogen in 100 uL volumes and stored at −70 degrees Celsius until the days of further experiments.

### Gold Incubation and Preparation of MBP Fusion Proteins

In the gold incubation experiments, samples of MBP fusions were incubated in 100 uL volume. For the fusion containing a single metallothionein the final concentrations during incubations were 50 uM protein, 10 mM disodium aurothiomalate, and 25 mM Tris-HCl pH 7.5. Likewise, for incubations with the fusion containing the dual metallothionein fusion, the concentrations were 50 uM protein, 20 mM disodium aurothiomalate, and 25 mM Tris-HCl pH 7.5. These concentrations provide a 20 to 1 ratio of aurothiomalate to cysteine. Control samples of identical volume and concentration were prepared similarly, however without the addition of the aurothiomalate. Samples were incubated for 3 hours at 37 degrees Celsius. After incubations were complete, samples were desalted by running the sample mixture over a Superdex10/30HR column (Pharmacia, Piscataway, N.J.) on an Akta FPLC (Pharmacia) with a 100 mM ammonium acetate buffer. Fractions were collected with 0.5 mL fraction volumes using a 0.5 mL/min flow rate, and sample elution was monitored with the UV detector set at a wavelength of 280 nm.

### Electrospray Ionization Mass Spectrometry

For ESI mass spectrometry, samples were desalted by buffer exchange in a spin concentration device to provide better ionization and the collection of clean spectra. First, samples were concentrated to 50 uL by spinning the samples within YM-3 microcentricons (Amicon, Beford, Mass.) at 12000×G in a tabletop microfuge. Desalting was performed by repeated dilution to 500 uL in 25 mM Tris-HCl followed by re-concentration to 50 uL in the YM-3 microcentricon two times. This was repeated 3 more times using a suitable ESI mass spectrometry buffer. For these experiments, the buffer was a 5 mM ammonium acetate buffer pH 6.0. Finally, samples were diluted to 100 uL with 5 mM ammonium acetate buffer pH 6.0 with 40% methanol to give a final concentration of 20% methanol in the samples. For an apo-metallothionein control, a small volume (less than 5 ul) of 0.1 M acetic acid was added to a Zinc-7-metallothionein sample to lower the pH below pH 4 and cause release of zinc from the protein. All samples were spun through a 0.4 um spin filter (Amicon).

All ESI mass spectrometry samples were run at the Brandeis University Biochemistry Core Facility on a Perseptive Biosystems Mariner (Framingham, Mass.). Samples were typically diluted an additional 5 or 20 fold at the time of recording in a 5 mM ammonium acetate buffer pH 6.0 with 20% methanol to obtain a strong signal with minimal protein amounts. Samples were injected at a rate of 3-5 uL/minute and collected over a mass to charge range of 500 to 4000. The nozzle and detector potentials were adjusted to obtain strong clean spectra. All samples were collected in positive ion mode. The instrument calibration was verified on the day of the experiment using apo-myoglobin (A8673, Sigma Chemical Corporation) in a 5 mM ammonium acetate buffer pH 6.0 with 20% methanol.

MALDI Mass Spectrometry of Fusion Samples

After desalting incubated sample, MALDI mass spectrometry was performed. All MALDI mass spectrometry samples were run at the Brandeis University Biochemistry Core Facility on a PerSeptive Biosystems Voyager (Framingham, Mass.). On the day of the experiment, fresh matrix solution was prepared. One of two matrixes was used for experiments. Either a 5 mg/mL sinapinic acid (SA) (D7927, Sigma Chemical Corporation) solution in a water:acetonitrile (50%:50%) or a 10 mg/mL 6-azo-thiothymadine (ATT) (27, 551-4, Aldrich Chemical Company) in a water:acetonitrile (50%:50%) were used as matrixes. Samples were diluted 10 or 20 fold in matrix solution to obtain a strong signal with minimal protein amounts. Samples diluted in matrixes were then spotted onto the sample plate with 2 uL per well. Droplets were allowed to dry and then placed into the mass spectrometer. Spectra were collected with a 25,000 V acceleration voltage in positive ion mode usually using a range of 20,000 to 100,000 mass/charge ratio. If samples did not yield strong signal, a portion of the desalted sample was concentrated in a Savant vacuum concentrator (Holbrook, N.Y.) and retested in the MALDI mass spectrometer.

### STEM of Fusion Protein Samples

Some of the desalted MBP fusion samples were sent for STEM imaging at Brookhaven National Laboratories. Samples were sent flash frozen in liquid nitrogen and shipped on dry ice. Samples were mixed with a small amount of Tobacco Mosaic Virus for use as a mass standard during imaging. These samples were defrosted and applied to grids containing thin carbon foils. Grids were rinsed with ammonium acetate buffer, and excess buffer was wicked away. This was followed by flash freezing and by a slow overnight freeze-drying of the grids to remove remaining buffer. Grids were imaged at 40 keV within the STEM. Images containing 512×512 pixels were collected using both the high angle and low angle dark field annular detectors.

### Transmission Electron Microscopy of Fusion Samples

Desalted samples of MBP fusion proteins within ammonium acetate buffer were saved for viewing within the transmission electron microscope (TEM). Quantifoil (Jena, Germany) grids with 1 micron diameter holes were used to support thin (<200 Angstrom) continuous carbon foils. Carbon foils were prepared by depositing carbon onto freshly cleaved smooth mica in an Edwards (West Sussex, UK) carbon evaporator. Thin foils were then floated on water and pieces of the foil were picked up on to the Quantifoil grids. The grids were seta side to dry for at least 24 hours before proceeding. Grids were glow discharged and 3 ul of sample was applied to the grid on the thin carbon surface. After 30 second the grids were rinsed twice with ammonium acetate buffer. Excess buffer was carefully blotted with Whatman sent, UK) filter paper from the edge of grids as to not touch the viewing area. The grids were allowed to fully dry before placing them within the TEM.

### Results

The purification procedure provided by New England Biolabs with the pMal system worked well for the metallothionein fusions. However, two modifications were made. First, no EDTA or other chelators were added at any step during the procedure. This was to prevent removal of metal atoms from metallothionein. The second modification was the use of 2-mercaptoethanol at a concentration of 0.1 mM. In purification procedures with metallothionein, it is common to use 2-mercaptoethanol over stronger dithiol-type reducing agents due to the chelating ability of dithiols [71]. The lower than suggested 2-mercaptoethanol concentrations and the decision to not use a non-thiol based reducing agent such as Tris-carboxyethylphosphine (TCEP) were chosen as to not interfere with subsequent reactions of the purified protein with gold compounds. Typically, induction with 0.2 mM IPTG for two hours caused development of a strong band at the correct molecular weights for the fusion proteins and led to purification of about 10 mg of fusion protein per liter of culture.

The goal of the protein expression was to produce a fusion protein that was as similar as possible to native, functional metallothionein. Under native conditions, metallothioneins are found with bound zinc, which makes them more insensitive to oxidation [68]. Hence, for this work, it was desired to purify metallothioneins loaded with zinc. Bacterial cells expressing metallothionein show increased tolerance to metal exposure, including zinc [72]. This suggests that excess zinc can penetrate into bacterial cells. Therefore, growth cultures were supplemented with 200 uM zinc sulfate a half hour after induction.

To quickly evaluate whether this supplement had any effect, purified MBP-MT protein grown in the presence of zinc, cadmium, or without additional metal was subjected to ESI-mass spectrometry. We found that metallothionein may be unable to complete fill its metal binding sites when produced within bacterial expression systems. We used ESI-mass spectrometry for this examination and the high degree of charging of the fusion protein allowed for the collection of peaks within the limited mass to charge range of the instrument. Spectra typically showed mass to charge peaks corresponding to 13 to well over 30 charges. This high degree of charging may affect the metal composition bound by the protein.

The apo-MBP-MT protein has an expected calculated mass of 48682 amu. The deconvoluted masses calculated from a mass to charge range of 2000 to 4000 for the zinc, cadmium, and no metal growth conditions all show an onset of mass at about 48650 amu, and the no metal growth condition spectrum shows a strong peak at 48667. These values agree well with the expected mass of the apo-protein. This suggests that in all cases, some of the protein does not contain metal atoms. The peak shapes of the collected raw spectra are identical to the various peak shapes observed in the deconvoluted spectra. Therefore, we can be confident that these shapes result from the data and not improper deconvolution. In addition, it is worth noting that the spectrum observed for the zinc supplemented growth condition is broad and does not contain sharp peaks like those observed in the other conditions. This may suggest that the sample may need further desalting. However, the high degree of charging observed argues this is not the case.

Comparing the deconvoluted spectra indicates that supplementing the growth media with metals may have an effect. As mentioned above, the majority of the protein grown without supplemented metal appears to be in an apo-protein state. Conversely, spectra observed for samples supplemented with zinc or cadmium show shifts towards increased mass. Given the observed mass of the apo-protein in these experiments, Zn-metallothionein with seven zinc atoms would have an expected mass of 49102 amu. Likewise, Cd-metallothionein with seven cadmium atoms would have an expected mass of 49382 amu. Under each metal supplemented condition, spectra indicate peaks corresponding to metal attributable masses less than these expected metal filled values. This may suggest that even with metal supplementation of the growth media, all metal binding sites may not be filled. However, these data suggest a fair amount of metal makes its way to metallothionein's binding sites, and as such may be beneficial for protecting the oxidation state of the cysteines.

Purified MBP metallothionein fusions (both the MBP-MT construct with one copy of metallothionein and the MBP-MT2 construct with two copies of metallothionein) were incubated with aurothiomalate to test their abilities to bind gold. These incubations were performed with an aurothiomalate to cysteine ratio of 20 to 1 to ensure full labeling of the fusion protein. Unlike the unfused metallothionein samples discussed in Example 1, desalting of gold-incubated samples by buffer exchange using a centrifugal concentrating unit was not often possible since the protein appeared to stick to the device's membrane. This was especially true with the dual-metallothionein construct. Instead, a Pharmacia Superdex 1030HR size exclusion column was employed to desalt samples.

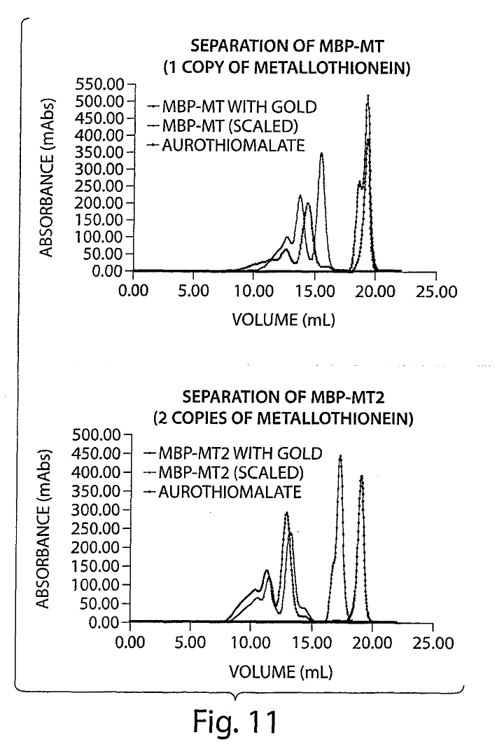

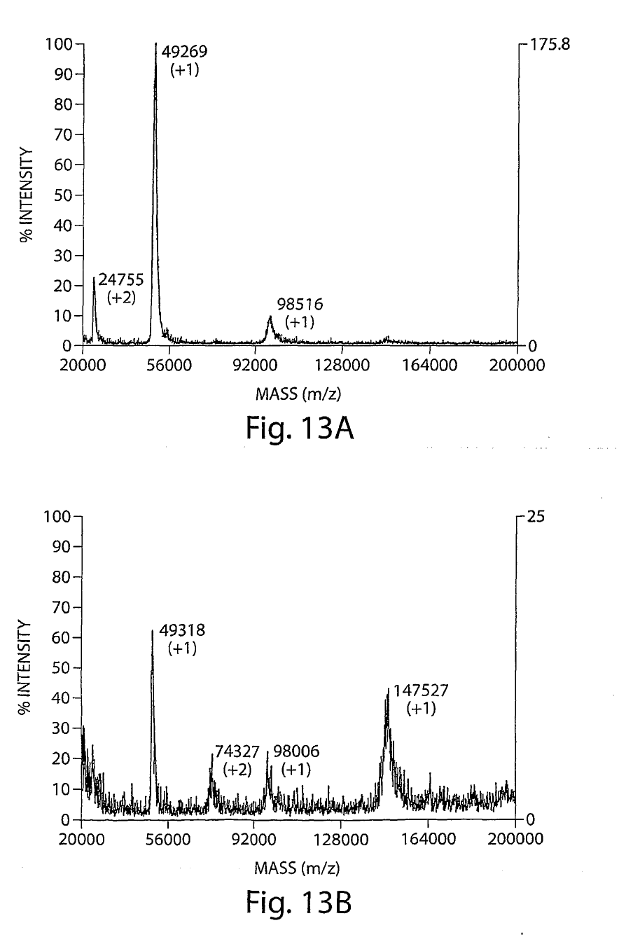

Typical results for a series of FPLC separations of the MBP-MT construct (top graph) and MBP-MT2 construct (bottom graph) recorded at an absorbance of 280 nm is shown in FIG. 11. The red traces in each graph are protein samples without gold. These traces show a series of peaks corresponding to different oligomeric states. The largest, and most well resolved peak in each red trace is the monomer peak. This was confirmed by SDS-page separation (result not shown) and MALDI mass spectrometry (see FIG. 13). As a control in each graph, aurothiomalate incubated at the same concentration as within the protein-incubated samples was run through the column, and these samples are shown in green. The aurothiomalate elutes as a strong, single peak well after the monomeric native protein peak. The traces of MBP-MT (top graph) and MBP-MT2 (bottom graph) incubated with gold are shown in blue. Like the native protein, gold-incubated MBP-MT2 protein elutes as a series of peaks corresponding to different oligomeric states. Two notable differences are observed. First, gold-incubated samples show a 2.5 to 3 fold increased absorbance at 280 mm for the series of elution peaks. Second, the peaks corresponding to the various oligomeric states elute slightly sooner when gold in present in reactions. This may suggest an increase in the Stokes radius upon gold binding. In addition to gold-bound protein peaks, a large, very slow elution peak with a slight shoulder is observed during separation of these gold-incubated samples. The relative elution positions of these peaks, as compared to the aurothiomalate controls, suggests these peaks most likely contain unreacted aurothiomalate and freed thiomalic acid. The reason for the slight shift of this peak in the MBP-MT2 gold incubated sample is unknown, but given this peaks position with respect to the resolving capabilities of the column, this change does not signify a large change in Stokes radius.

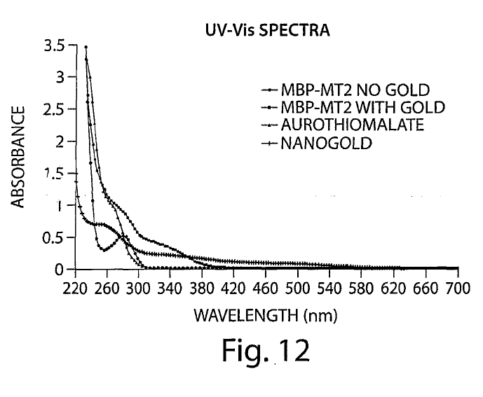

Given the sizable increases in the 280 nm absorbance measurements of gold-incubated samples as compared to native protein samples, the absorbance spectra of the monomeric FPLC peaks were examined. Samples of the native protein and gold-incubated protein after separation are shown in FIG. 12 colored in red and green, respectively. For comparison, spectra for aurothiomalate and Nanogold are also shown in blue and black, respectively. All spectra show large absorbance values around 220 nm. This is most likely due to the sizable sulfur and gold contents that are known to absorb in these regions. As expected, the native protein has a characteristic 280 m peak. Both the aurothiomalate and native protein fall to undetectable levels by 300 nm. By contrast, the gold-incubated and Nanogold® spectra contain a large extended shoulder past 300 nm. This common feature and the absence of such a shoulder in the aurothiomalate spectrum suggest this shoulder derives from gold cluster formation. However, any interpretation of gold absorption spectra is difficult without detailed structural information [26].

Although the various oligomeric states were resolved on an SDS-PAGE gel (data not shown), a secondary means of verification of the oligomeric state was desired. This was necessary since interpretation of imaged samples relies greatly on knowing the exact sample composition. A comparison of MALDI mass spectra for two different oligomeric peaks of native MBP-MT protein are shown in FIG. 13. Panel A shows the monomeric fraction. Three peaks are evident in the spectrum. The first two, at 24755 amu and 49269 amu, correspond to the monomeric MBP-MT with a +2 and +1 charge, respectively. The small third peak at 98516 amu is due to a small amount of dimer in the sample. Alternatively, mass spectrum of a fraction containing the trimer peak is shown in panel B. This spectrum has two relatively stronger peaks at 74327 amu and 147527 amu, which are due to protein trimers with a +2 and +1 charge, respectively. The weak signal component in Panel B is a likely consequence of the lower concentration of protein within separated fractions and the limited detection of mass spectrometry instrumentation with increased sample mass. Unfortunately, it has not been possible to collect mass spectra of trimer samples of MBP-MT samples incubated with gold. Like the native trimer sample, the expected signal would be weak, yet the characteristic peak broadening witnessed upon gold binding most likely causes the signal strength from these peaks to be below the detection limit of the instrument. Hence, no detectible spectra are expected. Nevertheless, the obtained mass spectrometry results verify the compositions of the various sizing column fractions.

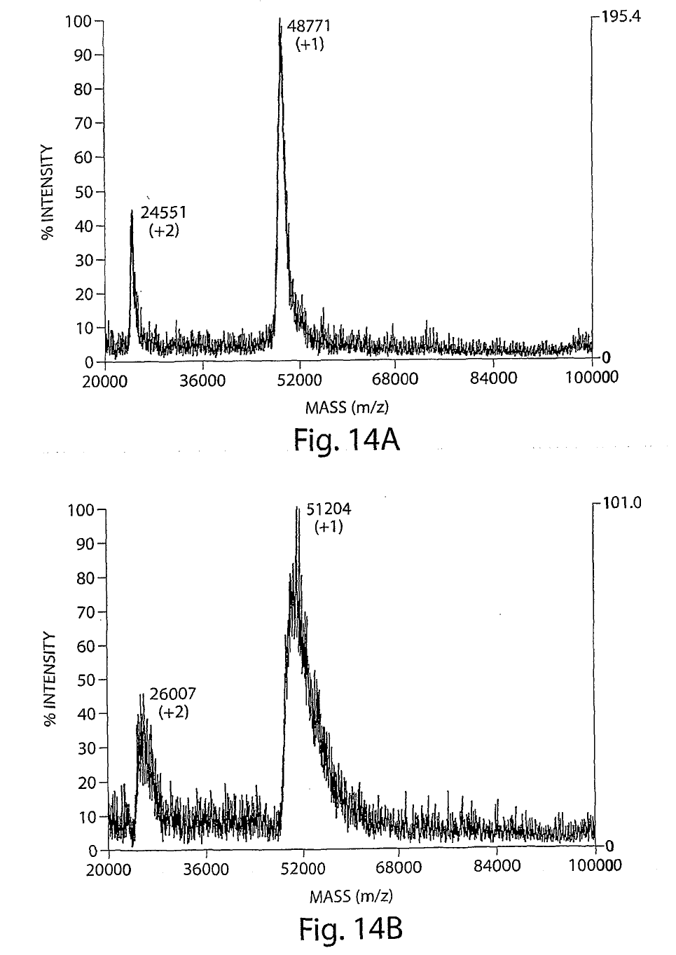

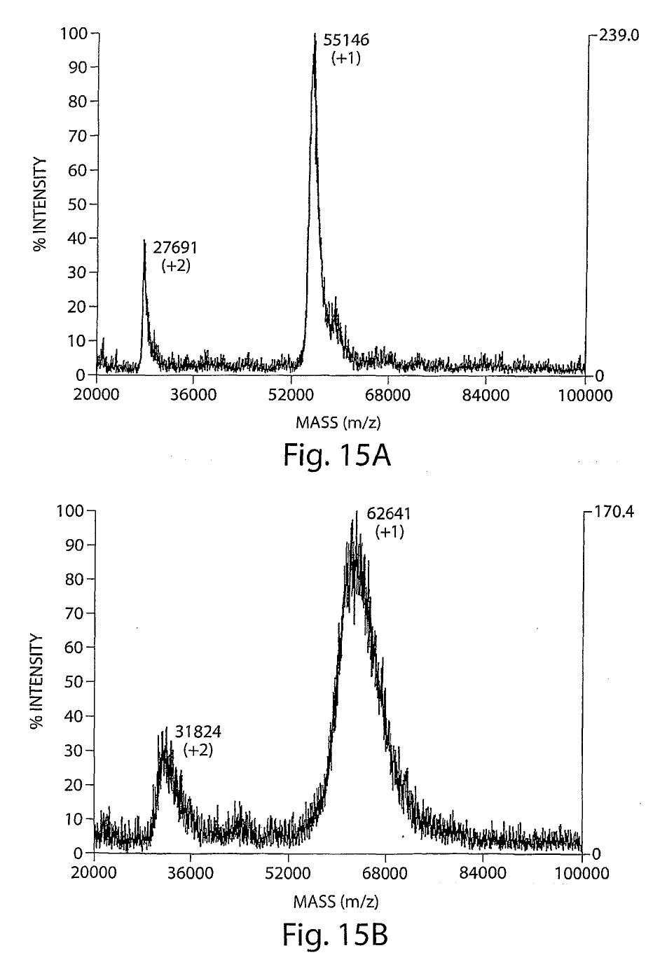

To evaluate gold binding, desalted monomeric peaks were analyzed for increased mass resulting from gold binding. These results are shown in FIGS. 14 and 15 for the MBP-MT and MBP-MT2 constructs, respectively. Panel A in each figure shows the native protein. As mentioned earlier, the MBP-MT fusion protein has an expected molecular mass of 48682 amu. Similarly, the MBP-MT2 fusion protein has an expected molecular mass of 55146 amu. The observed masses of 48771 amu and 55146 amu in FIGS. 14 and 15, respectively, are in good agreement. One explanation for the minor mass differences of the observed and expected values may be the presence of bound metal atoms as suggested earlier with ESI-mass spectrometry data. Some metal atoms common within cells, such as copper, may not be removed under the conditions used for preparing MALDI samples. Panel B in each figure shows the gold-bound monomeric fraction for each construct. For the MBP-MT fusion protein, a shift to 51204 amu is indicated. The mass difference between the native and gold-bound states would indicate that about 12 to 13 gold atoms have been bound at the new peak position. Similarly, the MBP-MT2 gold-bound state shows a molecular mass of 62641 amu. This difference would signify about 38 gold atoms bound to this dual metallothionein construct. Typically, results for these mass spectrometry experiments were comparable to the low gold-bound state discussed above in Example 1 where 15 to 17 gold atoms are bound for each metallothionein copy in a fusion protein.

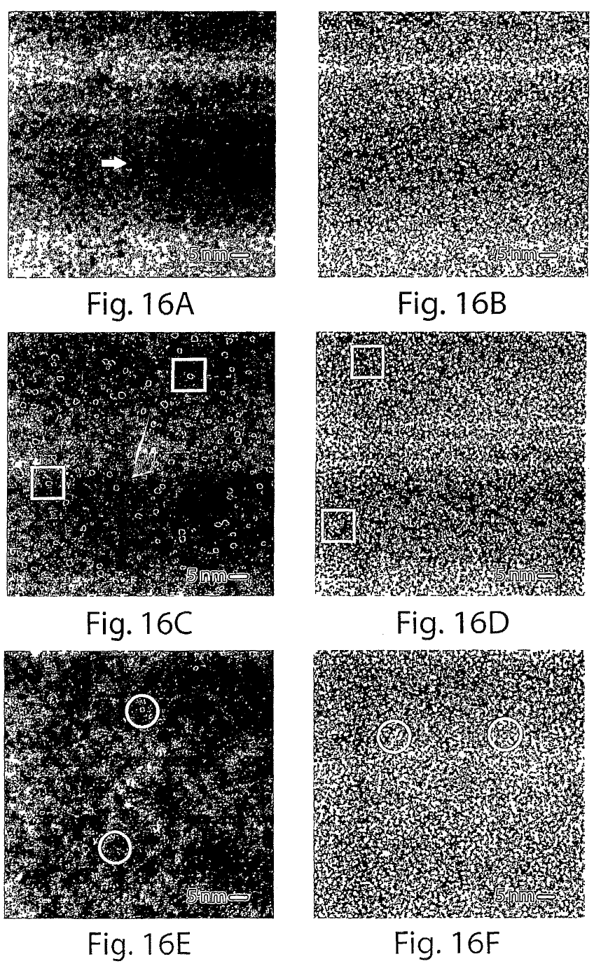

After the separation of the various oligomeric states and an evaluation of the distribution of gold binding, it was possible to confidently interpret visualized MBP metallothionein fusions. A combination of STEM and TEM data are shown in FIGS. 16, 17, and 18. In each figure, a STEM and a TEM image were prepared from the same sample and are displayed next to each other. STEM samples are shown in the left columns of each figure while TEM images are shown in the right columns. Only the high angle dark field STEM images are displayed in these figures. Furthermore, the intensities of STEM images have been inverted to make clusters easier to visualize. In addition, a constant contrasting factor has been assigned to further aid visualization of these STEM images. Assurance that this does not distort interpretations from these images comes from the comparable background levels and fluctuations observed in all STEM images shown. TEM images were collected with a small defocus value of about −400 nm. This is necessary to provide suitable phase contrast with minimal blurring of the collected image. Equivalent amounts of defocus between images were obtained by comparing calculated 2 dimensional Fourier transforms of collected images using the microscope's CCD camera. All displayed images have been scaled to equivalent sizes.

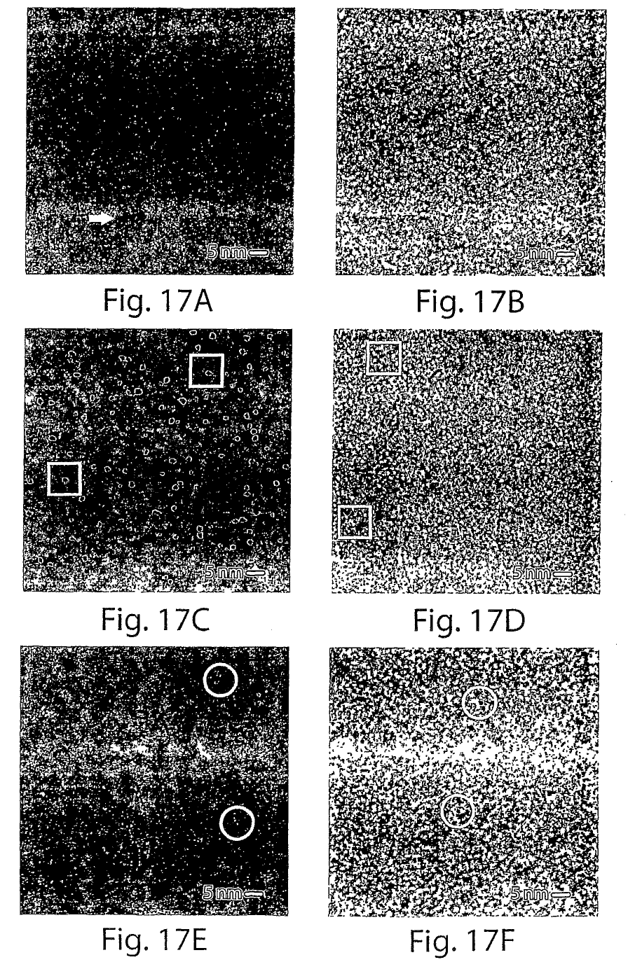

In FIGS. 16 and 17, STEM and TEM images of Nanogold® are displayed in Panels C and D, respectively. This acts as a positive control for comparing the sizes and scattering abilities of gold clusters formed by the MBP-metallothionein fusion proteins. As a reminder, these commercial clusters are believed to contain between 55 to 65 gold atoms, and have an expected diameter of 1.4 nm. Examples of individual Nanogold® clusters observed with STEM and TEM are located within the centers of the blue squares. In both forms of imaging, Nanogold® clusters appear rather uniform in size and as strongly scattering objects. The results obtained provide qualitative evidence for metallothioneins use as a TEM label.

The MBP-MT fusion protein containing only one metallothionein gene is compared in FIG. 16. As a negative control, the MBP-MT fusion protein incubated without gold is shown in panels A and B. Within the STEM image in Panel A, a faint, weakly scattering signal, which is believed to be protein without gold, is designated by a yellow arrow. Protein visualized by STEM and TEM are suspended on a thin carbon foil. Since this carbon foil is slightly denser and about as thick as an MBP metallothionein fusion protein, the signal from protein on top of the carbon will not be very different from the carbon foil alone. This weak signal usually places the lower limit of detection by STEM at 50 kD, which is about equal to the proteins visualized here. This ability of STEM to visualize such unstained proteins of this size should be superior to TEM since dark field STEM image contains signal mostly from elastically scattered electrons. TEM images arise from a combination of inelastically scattered, elastically scattered, and unscattered electrons. On top of this, the strong contrast transfer function of the TEM introduced by defocusing the image adds a frequency-dependent contrast effect. This makes TEM images more grainy. Hence, these conditions of image formation make TEM images comparably nosier than STEM images. Thus, in the TEM image shown in panel B, a similar weak signal comparable to the STEM data can occasionally be found. These are most likely due to closely packed proteins rather than image signal from a single monomer.

Gold-incubated MBP-MT proteins are displayed in panels E and F. Similar to the Nanogold®, the STEM image in panel E shows small, strongly scattering regions most likely resulting from the bound gold. Examples of single clusters formed by MBP-MT can be seen in the centers of the red circles in the STEM and TEM images. With the STEM image, cluster sizes appear to range up to as large as 1 nm in diameter. However, these clusters appear much less uniform in size than the Nanogold® clusters. This is not unexpected given the mass spectrum displayed in FIG. 14 Panel B that shows a large distribution of masses. Panel F displays the TEM image of an MBP-MT sample incubated with gold. Unlike the Nanogold, sample, visualizing the MBP-MT sample with gold is much more difficult with the TEM. Only the largest clusters within the sample are evident in the images. Small clusters may be obscured by the high noise level and image modulations induced by defocusing as discussed above. Nevertheless, gold clusters formed by MBP-MT can be seen inboth STEM and TEM.

Electron microscopy images of the construct containing two copies of metallothionein fused to MBP are displayed in FIG. 17. Like the MBP-MT construct, the negative control images in Panels A and B show a relatively weak signal from protein without gold as designated by the yellow arrow. The STEM and TEM images of the gold-incubated sample are shown in Panels E and F. The STEM image shows regions of speckling. Many of these appear to have two to four small, strongly scattering spots as shown in the red circles. This suggests that gold-incubated MBP-MT-2 complexes may not form distinct single clusters. It is interesting to speculate that these may actually be small gold clusters bound to individual metallothionein units or metallothionein domains. Strikingly different are the clusters observed in the TEM image of Panel F. These images of gold clusters formed by MBP-MT2 appear very uniform and at times larger than Nanogold®. On average, these clusters appear to be 1.4 nm in diameter. The very larger clusters, approaching 2 nm in diameter, may be formed by single MBP-MT2 proteins or possibly by two gold-bound MBP-MT2 proteins in close proximity. Most noteworthy from these images is that clusters formed using a dual concatenated metallothionein construct are clearly as visible as Nanogold®.

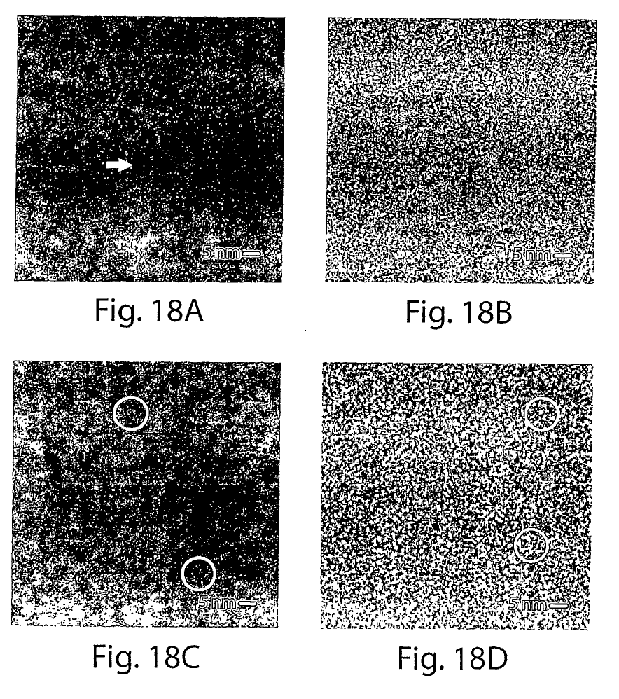

To get a better idea of the exact nature of metallothionein copy number's effect on the size of gold clusters, the trimer fractions from desalted MBP-MT2 gold-incubated mixtures were imaged. These results are shown in FIG. 18. As in the previous two figures, the negative controls of protein incubated without gold are shown in Panels A and B. Again, only weak signal due to protein alone is witnessed in the STEM and TEM images. The STEM images in Panel C show large densities on the order of 5 nm. These still do not appear as dense as Nanogold®, but rather they look more speckled like the STEM images of the MBP-MT2 monomers, though more tightly packed together. More interesting is the TEM image shown in Panel D. Clusters appear to be aggregated into groups of two and three speckles. Examples of these are shown in the centers of the red circles in Panel D. Several facts lead to the conclusion that these groups of clusters are MBP-MT2 dimers and trimers. First, grid samples were made from purification fractions well characterized as protein trimers. These trimer fractions eluted from the sizing column with a mobility and elution profile consistent for that expected for an oligomer composed of three MBP-MT2 fusion proteins. This covalent oligomeric state was further confirmed by SDS-PAGE and MALDI mass spectrometry. Second these cluster groups are well separated on the grids so crowding within the image is not a problem. Finally, it is stunningly apparent that the groups contain two to three individual clusters as would be expected for dimer or trimers not associating in a single large cluster. Thus, each individual cluster in the trimer most likely results from one MBP-MT2 protein. The individual clusters in this image are slightly larger than imaged Nanogold® with diameters of about 1.4 nm.

### Example 3

### Imaging of Antibody-Labeled Gold-Bound Fusion Protein Complexes

For cryo-electron microscopy, a theoretical lower mass limit of about 100 kDa has been calculated [2]. Therefore, metallothionein, by itself, is not suitable for visualization by this method. To circumvent this limitation, gold-labeled MBP-MT2 complexed with another protein was used. A simple protein complex consisting of an MBP antibody with various MBP-MT2 preparations bound to each of its antigenic binding sites was examined. Specifically, a commercially available monoclonal IgG2a MBP antibody and the previously described MBP-MT2 protein from Example 2 with and without gold were used. Hence, the augmented mass of the combined complex is about 260 kDa Although this is above the theoretical limit, this mass value places the antibody complex in the lower range of proteins analyzed to date by cryo-electron microscopy using single particle methods which is around 250 kDa [76].

Recently, there has been a report of IgG antibodies imaged by electron tomography [77]. Independent groups have solved x-ray crystal structures for complete IgG antibodies and atomic coordinates are available for the three domains of this molecule. However, the crystal packing of individual molecules has shown variable orientations for domains [78]. Likewise, the electron tomography work highlights that imaged antibodies are extremely flexible. The electron tomography data also shows antibodies flash-frozen in solution appeared with a characteristic Y-shape composed of three domains. Two ellipsoid domains are believed to be the two Fab arms and a heart-shaped domain is most likely the remaining Fc region [77]. While raw images were not provided in the tomography work, the 3-dimensional reconstructions do provide an idea of the general shapes available for these flexible structures. In addition, the ability to visualize these small antibody molecules without bound antigen gives confidence that the preliminary results presented in this work with gold-bound MBP-MT2 attached to an IgG are accurate.

Here we present results of MBP-MT2 antibody complexes imaged by conventional stain-contrast TEM, cryo-electron microscopy, and STEM. Initial imaging in cryo-electron microscopy displays protein complexes that resemble what would be expected for antigen-antibody complexes with gold.

### Materials and Methods

### Preparation of MBP-MT2 Antibody Complexes

Prior to incubation of MBP-MT2 with the monoclonal MBP antibodies, both proteins required preparation. The MBP monoclonal antibodies were purchased form New England Biolabs (Bedford, Mass.) and were provided in 50 percent glycerol solution as a 1 mg/mL solution. Antibodies were subjected to buffer exchange using a 10 kD MWCO microconcentration device (Pall, East Hills, N.Y.). The antibodies were resuspended in TBS buffer in a final volume of 500 uL and re-concentrated to 50 uL. This combination was repeated two more times. Preparation of the MBP-MT2 protein began with separation of the different oligomeric species as described in Example 2. A gold-incubated sample and control sample without gold were prepared. As a final step, the monomeric peak fractions for each sample were combined and concentrated to 20 uL within a Speedvac (Savant, Waltham, Mass.) vacuum concentrator.

Antibody complexes for both the control and gold-incubated MBP-MT2 samples were formed by mixing 25 uL of concentrated antibody with 20 uL of MBP-MT2 protein and 10 uL of TBS buffer. These samples were incubated at room temperature for 1 hour. After 1 hour, the samples were separated on a Pharmacia 3.2/30 Superose 12 column using a Pharmacia Akta FPLC. The buffer used for separations was 100 mM ammonium acetate and 100 uL fractions were collected while monitoring an absorbance of 280 nm. After separation, fractions were kept on ice until preparation of electron microscopy grids.

Imaging of MBP-MT2 Antibody Complexes

To ensure samples eluted from the sizing column contained complex, peak fractions were first prepared for TEM visualization in negative stain. For this, 400 mesh TEM grids supporting a thin carbon foil were prepared by negative glow discharging their surfaces in an EMitech (Kent, UK) glow discharge unit. Once prepared, 3 uL of sample was placed on the grid surface for 30 seconds. Then, the grids were stained with several drops of filtered 2% urnanyl acetate stain. After letting the stain sit on the grid for 30 seconds, excess stain was wicked away with Whatman (Kent, UK) #1 filter paper, and grids were allowed to completely dry before further work. Grids containing sample were visualized in a Morgagni TEM (FEI, Eindhoven, The Netherlands) using a 80 keV acceleration voltage. Images were collected on with a 2 k×2 k CCD camera (Hamamatsu, Japan).

A portion of the fractions containing antibody complex were prepared for cryo-electron microscopy on Quantifoil® R1.2/1.3 (Jena, Germany) holey grids. Grids were prepared by negative glow discharging their surfaces in an Emitech (Kent, UK) glow discharge unit. For freezing sample, three 5 uL drops of protein containing sample were placed on the grid with blotting with Whatman #1 filter paper between application of drops. After the final blot, the sample was quickly plunged into liquid ethane to vitrify the sample. Once frozen, the sample was transferred to liquid nitrogen and stored till the day of image collection. On the day of microscopy, the grid was transferred under cryo conditions to a pre-cooled Gatan (Pleasanton, Calif.) 626 single tilt cyro-holder. The grid was then transferred into a CM12 (Philips-FEI, Eindhoven, The Netherlands) TEM equipped with a low dose kit. Images were collected under low dose conditions with the microscope set at 120 keV aiming for a 1 nicron defocus. All images were collected on SO-163 Kodak (Rochester, N.Y.) film.

A second portion of the sample was sent to Brookhaven Nation Laboratories for STEM imaging. Samples were shipped overnight on wet ice and prepared by Martha Simon in the laboratory of Joseph Wall. Samples were placed onto grids containing thin carbon foils and rinsed with ammonium acetate buffer. Excess buffer was removed and samples were flash frozen followed by a slow overnight freeze-drying of the grid to remove excess remaining buffer. Grids were imaged at 40 keV within the STEM. Images containing 512×512 pixels were collected using both the high and low angle dark field detectors.

### Results

Prior to formation of complexes for imaging, the MBP antibody was assessed for its ability to interact with MBP-MT2. This was evaluated by incubating the antibody with MBP-MT2 protein. After incubation, agarose beads coupled to protein A were added, and an antibody pull down assay was performed. SDS-PAGE showed that the MBP antibody was able to pull native MBP-MT2 protein from the incubation mixture.

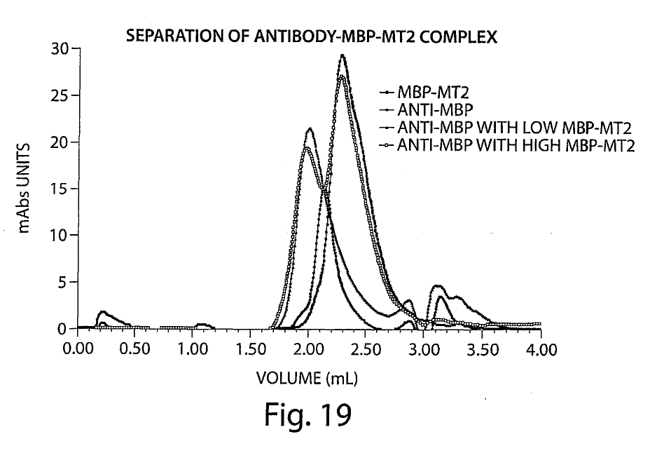

With an antibody-antigen complex size approaching the lower limit of cryo-electron microscopy, it is important to obtain as homogeneous a sample as possible. Therefore, a procedure to obtain a highly purified complex was developed. This procedure relies upon a Superose 12 size exclusion column for separation of MBP-MT2 antibody complex from uncomplexed antibody. However, in order to obtain satisfactory chromatographic resolution, the antibody solution was subjected to buffer exchange in order to allow better separation in the Superose 12 column. This is also beneficial for visualization since glycerol often interferes with proper grid staining and freezing. FIG. 19 shows the elution profiles of various protein containing samples. The MBP-MT2 protein alone and the MBP antibody alone are shown in blue and black, respectively. The two additional runs shown, correspond to antibody-antigen complex formation with nearly equal molar ratios of MBP-MT2 to antibody antigen binding sites (green) versus formation with excess MBP-MT2 at a ration of about 4:1 (red). These are shown in green are red, respectively. With its increased mass, the MBP-MT2-antibody complex travels more quickly through the column. This shows that it is possible to separate antibody complex from excess components. As expected, the profile for the sample of antibody incubated with excess MBP-MT2 shows development of two peaks, one with equal mobility to the MBP-MT2 and another peak at the mobility believed to be antibody-antigen complex. The redevelopment of a MBP-MT2 peak gives confidence that antigen binding sites on these antibody complexes are saturated. Therefore, eluted fractions from the antibody complex peak were used for imaging.

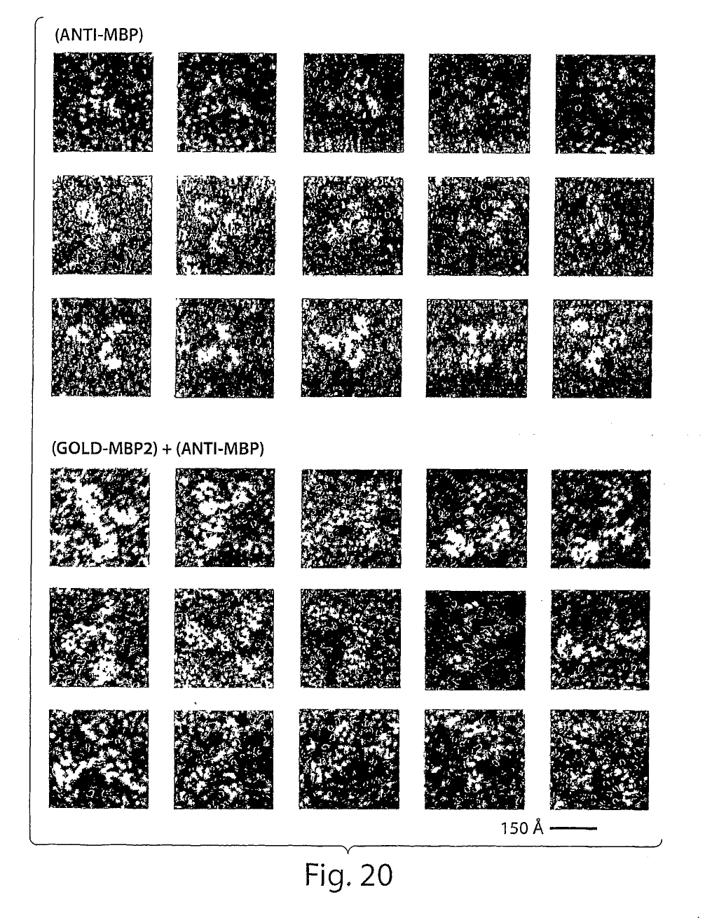

FIG. 20 shows a gallery of antibody complex formed with MBP-MT2′ incubated with gold. These protein complexes are stained with uranyl acetate to provide significant contrast. Nanovan®, which has often been used in conjunction with the commercial TEM labels, was tried, but it did not provide enough contrast to produce good images. This may result from the limited size of the proteins. Although in some images dense patches are present which are suggestive of label, it is impossible to clearly identify these as gold. Instead, dense patches could result from fluctuations in the stain layer around the antibody complex. As the literature suggests, the antibody complexes appear variable in shape. Comparison of the two sets of images in FIG. 20 clearly shows a difference in observed sizes between antibodies and antibody complex. The extended length witnessed on only two of the three domains agrees well with the idea that the MBP-MT2 protein has been bound at each of the antigen binding sites. This visualization in conventional stain-based TEM suggests it should be possible to examine these complexes with the inherently more difficult techniques of cryo-electron microscopy and STEM.

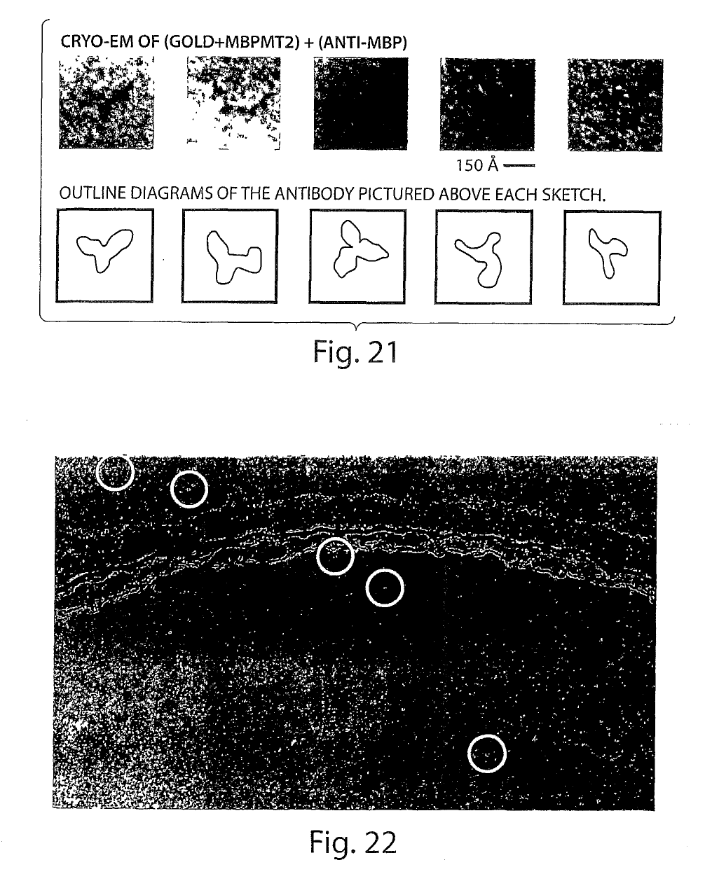

Initial trials at imaging antibody complexes in vitreous ice using cryo-electron microscopy techniques provided no data. Holes within the carbon films contained only a thin layer of fairly transparent ice, but no protein was evident. Conversely, the carbon films contained small, nanometer sized clusters reminiscent of the clusters observed in Example 2. This was solved by increasing the concentrations and volumes used to prepare grids. FIG. 21 shows a gallery of characteristic Y-shaped views obtained from micrographs collected using cryo-electron microscopy. These views are understandably rare since particles are randomly orientated within the ice and do not have an imposed directions as in the case of the stain-based TEM images of the complex. In some of these images dark regions can be seen near the ends of the complexes' arms. Although this is the expected location for the gold-bound domain, further work is needed to conclusively prove such a claim.

Given the limited number of views of antibody complex obtained, a simple examination of gold-labeled MBP-MT2 protein was also performed. An image of a cryo-electron microscopy micrograph of the gold-labeled MBP-MT2 protein is shown in FIG. 22. In the blue circles, examples of electron dense clusters believed to be that of gold-labeled protein are displayed. These can be seen on the carbon as well as suspended within the vitreous ice suspended in the grid's holes. These clusters appear of equal size to those shown earlier in Example 2. Although more work needs to be done, this shows gold-labeled metallothionein is viewable under low-dose cryo-electron microscopy conditions.

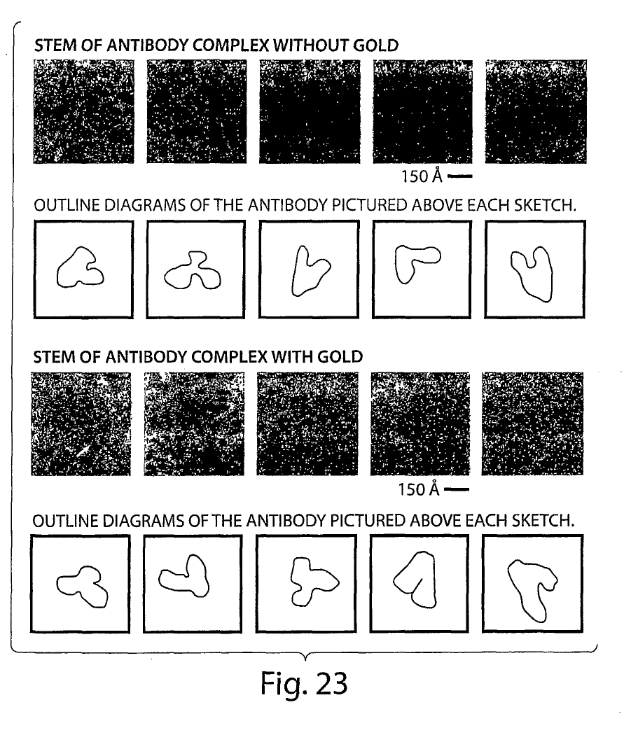

Several samples were sent for imaging by STEM, but complications have yielded only limited results. Grids often showed little material bound to their carbon surfaces which may indicate the need for more concentrated samples. FIG. 23 shows a gallery of antibody complexes formed with MBP-MT2 with (bottom) and without gold (top). As with the TEM images, the complexes appear flexible and variable in shape. Occasionally, dense patches (arrow) can be seen in the gold-bound samples that appear similar to results presented in Example 2. These may suggest the presence of gold clusters, but this is difficult to conclude given the limited data. Nevertheless, antibody complex can be seen, once again giving confidence to our methodology.

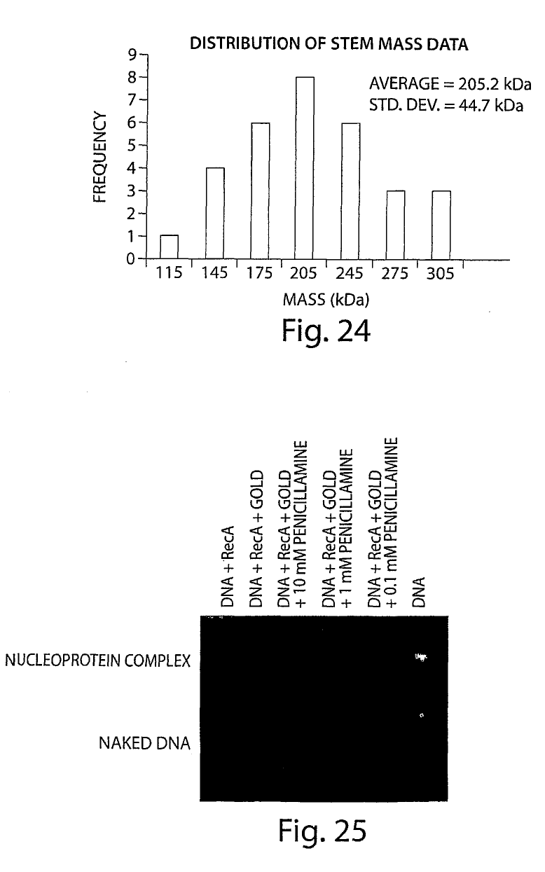

The STEM data have another useful quality in that it is possible to calculate masses of particles from the data collected. Based on the data obtained, a value of 205.2 kDa with a standard deviation of 44.7 kDa was calculated. As a reminder, the fully formed complex has an expected mass of about 260 kDa (150 kDa for the antibody and 55 kDa for each MBP-MT2 protein). This average value would agree best with a complex composed of an antibody molecule bound to only one MBP-MT2 protein. Furthermore, the large standard deviation may indicate that there are complexes with two MBP-MT2 proteins bound while some antibodies are bound to no other proteins. The distribution of measured masses is displayed in FIG. 24. This range is consistent with the presence of various bound states of the complex.

### Example 4

### Removal of Gold from RecA

Aurothiomalate and other gold compounds, which can deliver gold(I) to metallothionein, may act as a potent inhibitors to protein function. The primary mode of inhibition is through binding to cysteines within these proteins. In addition, a secondary mode of inhibition, when these compounds are used at relatively high concentrations (usually millimolar or greater), has been observed. This second form is most likely caused by electrostatic interaction to weak non-specific binding sites [79]. Although this second form usually appears reversible upon dialysis, the first form is not [80]. Therefore, a method for removing these tightly bound inhibitory gold compounds is desirable.

The exact mechanism of inhibition is directly related to gold chemistry. Gold(I) is relatively unstable, but it can be stabilized by soft electrophiles. Stabilization is usually accomplished through coordination to a thiol or phosphine compound. This is the function of the thiomalic acid portion of aurothiomalate [80]. While coordination to only one ligand can form a stable complex, often gold prefers linear coordination if excess ligand is available. Therefore, gold atoms often form polymers with atoms bridged between two stabilization ligands [79]. These ligands can be readily exchanged if more stable ligands are present [81]. This forms the basis for the reaction of these compounds with cysteines that are chemically more stable ligands than thiomalic acid. This increased stability arises because the thiolate of a cysteine is more electrophilic than that of a thiomalic acid. Thus, transfer is favored. Moreover, ejection of the thiomalic acid is extremely likely if two cysteines within a protein are in close enough proximity as to allow bridging [81]. In this manner, gold(1) compounds can inhibit proteins by binding to cysteines with or without removal of their thiomalic acid ligands.

RecA is the central component in the DNA repair and recombination pathways in E. coli, and homologues to this protein can be found in almost every organism [82]. Biochemical and TEM structural studies report that this protein forms a nucleoprotein complex able to coat a DNA strand with 1 subunit bound to every 3 to 4 base pairs [83]. Important to gold removal is RecA's 3 cysteines, located at position 90, 116, and 129. Each of these residues has been independently mutated to serine without loss of function [84]. However, replacement of all three has not been reported. Thiol reactive probes are accessible to all three cysteines [85]. Furthermore, cysteine-modified RecA proteins show inhibition of several functions, yet are reported to still bind single stranded DNA [84]. Although the protein may bind, the extent of nucleoprotein complex formation is unknown.

We have now shown aurothiomalate's ability to partially inhibit RecA function. Furthermore, penicillamine, which is another thiol containing compound often used as a medical treatment for metal poisoning and which has been demonstrated to remove gold atoms from proteins in vitro, has been shown to remove bound gold and reverse aurothiomalate-dependent inhibition [86]. Finally, the inability of penicillamine, incubated under the same conditions as with RecA, to remove all gold atoms bound to metallothionein has been shown. This highlights the unique chemical character of metallothionein and demonstrates penicillamine's potentially usefulness for removing superfluous gold atoms from fusion proteins.

### Methods and Materials

### Preparation of RecA Samples

To test aurothiomalate's inhibitory capacity, RecA protein was purchased from New England Biolabs (Beverly, Mass.) and assayed for nucleoprotein filament formation with a mobility shift assay. Prior to sample preparation, 100 uL of a 2 mg/mL solution of RecA protein was subjected to buffer exchange to remove dithiotreitol and glycerol. This was performed through successive dilution and concentration of the protein using a Pall 10 kD MWCO microconcentration centrifugal device (Ann Arbor, Mich.). After 3 rounds of buffer exchange with 25 mM Tris-HCl pH 7.5 corresponding to about 125 fold reduction in dithiotreitol and glycerol concentration, samples were prepared.

Five samples were prepared using the buffer exchanged RecA protein. First, 20 uL was placed in a separate tube as a control sample without gold. The remaining 80 uL was incubated with a final concentration of 1 mM aurothiomalate for 1 hour at 37 degrees Celsius. After this incubation, the sample was placed into another Pall 10 kD) MWCO microconcentration centrifugal device and buffer exchange as described above was used to remove unbound gold. This desalted protein was used to prepare the 4 remaining samples.

### Mobility Shift Assay

Mobility shift assay samples were prepared from the protein described above. In each sample, 40 ug of protein was mixed with 0.5 ug of 1000 base pair double stranded DNA in 25 mM Tris-HCl pH 7.0, 1 mM magnesium chloride, and 2.5 mM ATPγS. One sample was prepared from the RecA that was not incubated with gold as a positive control, and a sample of DNA alone was prepared as a negative control. The four samples prepared from gold-incubated RecA were supplemented with 0 mM, 0.1 mM, 1 mM, and 10 mM final concentration penicillamine. All samples were incubated for 1 hour at 37 degrees Celsius prior to running on a 0.8% agarose gel.

### Preparation of Metallothionein Samples for MALDI Mass Spectrometry

Samples of metallothionein were prepared by first incubating metallothionein with aurothiomalate to form gold-bound metallothionein. Specifically, 200 uL of a 1 mg/mL metallothionein supplemented with 20 mM aurothiomalate was incubated for 3 hours at 37 degrees Celsius. After the incubation, unbound gold was removed by desalting as described above except a Centrion YM-3 microconcetration device (Millipore, Billerica, Mass.) was used. The sample was then split in half One half was set aside as a positive control while the other half was supplemented with a 20 mM final concentration of penicillamine. These samples were incubated for an additional hour at 37 degrees Celsius followed by another round of desalting to remove components of the mixture not attached to the protein.

MALDI Mass Spectrometry All MALDI mass spectrometry samples were run at the Brandeis University Biochemistry Core Facility on a Perseptive Biosystems Voyager (Framingham, Ma). On the day of the experiment, fresh matrix solution was made. One of two matrixes was used for experiments, either a 5 mg/mL sinapinic acid (SA) (D7927, Sigma Chemical Corporation) solution in a water:acetonitrile (50%:50%) or a 10 mg./mL. 6-azo-thiothymadine (ATT) (27, 551-4, Aldrich Chemical Company) in a water:acetonitrile (50%:50%). Samples were diluted 10 or 20 fold in matrix solution to obtain a strong signal with minimal protein amounts. Samples diluted in matrixes were then spotted onto the sample plate using 2 uL/well. Droplets were allowed to dry and then placed into the spectrometer. Spectra were collected with a 25,000 V acceleration voltage in positive ion mode over a range of 1500 to 50,000 mass/charge ratio.

### Results

In order to evaluate RecA function, a simple mobility shift assay was chosen. Since RecA can coat double stranded DNA in the presence of ATPγS to form a nucleoprotein complex, the mobility of these complexes upon electrophoresis will be greatly retarded. FIG. 25 shows an ethidium bromide stained 0.8% agarose gel containing various RecA incubated samples. Lane 1 (leftmost) shows the retarded mobility of the control RecA complex with DNA as compared to lane 6 that shows a sample containing only DNA. Lane 2 shows the altered mobility of DNA within a nucleoprotein complex formed with RecA that was incubated with aurothiomalate. A smear ranging in mobility from the size of naked DNA to almost the size of the fully decorated nucleoprotein complex can be seen. Although this smear suggests some degree of protein binding to the DNA, it is not equivalent the RecA control in Lane 1. Thus, aurothiomalate has a deleterious affect on RecA protein function. Reversal of this inhibition can be seen in the three RecA incubated gold samples mixed with penicillamine. Lanes 3, 4, and 5 show final penicillamine concentrations of 10 mM, 1 mM, and 0.1 mM, respectively. These concentration correspond to molar ratios of penicilamine to cysteine in reactions of about 100 to 1, 10 to 1, and 1 to 1, respectively. As the penicillamine concentration is increased, a trend towards function more equivalent to non-gold reacted RecA is witnessed. Since it is expected that aurothiomalate binds to RecA's cysteines, this suggests that penicillamine is able to remove these bound ligands.

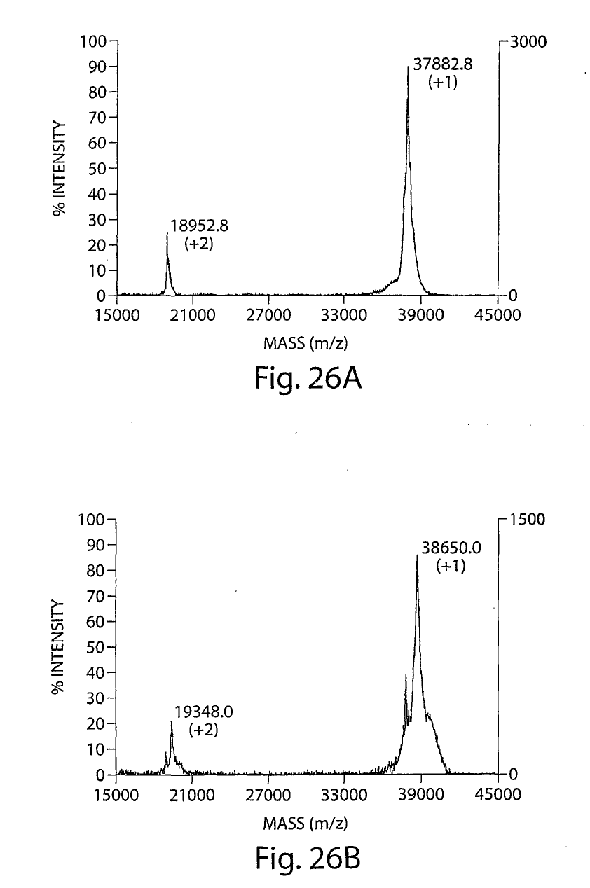

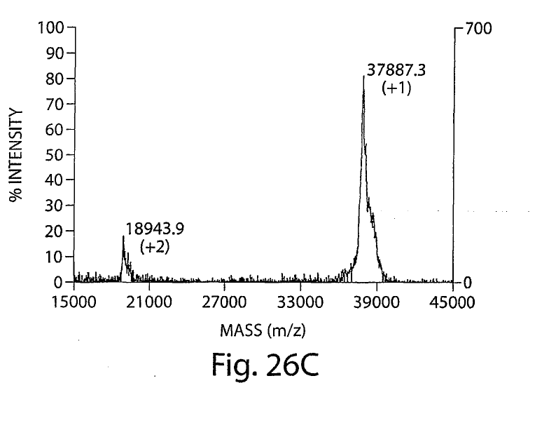

As a way to verify both binding by aurothiomalate and removal by penicillamine, MALDI mass spectrometry was used to examine samples. FIG. 26 shows spectra collected from three different RecA samples. All samples showed strong well-resolved peaks. Panel A shows the control spectrum collected from RecA that was not incubated with aurothiomalate. The +1 mass to charge peak at 37,883 amu is in good agreement with the expected RecA molecular weight of 37,842 amu [83]. Panel B displays a RecA sample that was incubated with aurothiomalate. As expected, the peak mass value shifts to a higher mass value. A new main peak is found at 38,650 amu showing an increase of 767 amu from the apo-protein state. Looking more closely at this spectrum, there appears to be a smaller, but still noticeable peak at the apo-protein mass value suggesting some protein has not bound aurothiomalate. The observed difference of 767 amu is difficult interpret. This difference is considerably larger than the value of 591 amu expected for 3 gold atoms bound without their thiomalic acid ligands. Likewise, if only 2 aurothiomalate groups were bound (with the retention of their thiomalic acid ligands), there would be an expected difference of 692 amu. A third possibility is that the main peak may correspond to 2 gold atoms along with 1 gold aurothiomalate ligand. This combination would give a difference of 740 amu. Whatever the case, the extra mass associated with the decreased reactivity of aurothiomalate-incubated RecA can be detected.

A spectrum collected from the same gold-bound RecA sample as in Panel B but with an additional treatment with 10 mM penicillamine in shown in Panel C. This sample shows a decrease in mass to a value of 38,887 amu. This is almost exactly equal to the apo-RecA control shown in Panel A. A slight extra shoulder on this peak within its higher mass slope may indicate that not all gold may be removed. However, the observed shift shows the expected removal of extra aurothiomalate associated mass. Moreover, this result agrees well with the functional mobility shift assay results shown in FIG. 25.

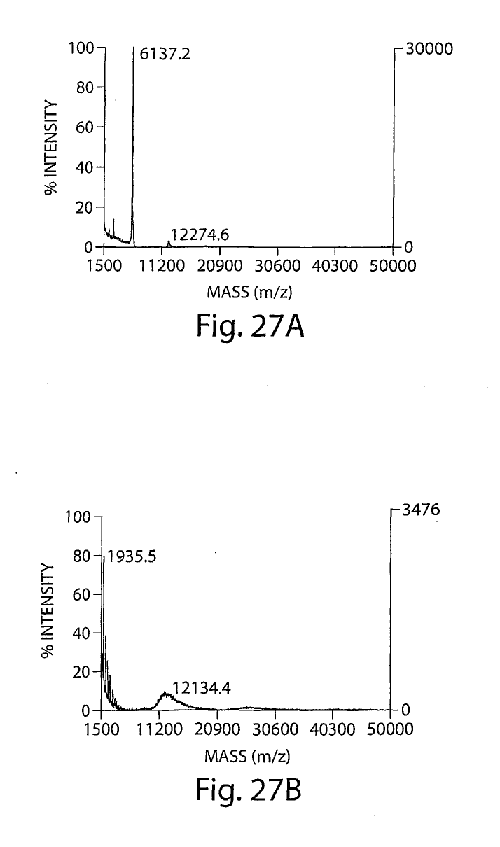

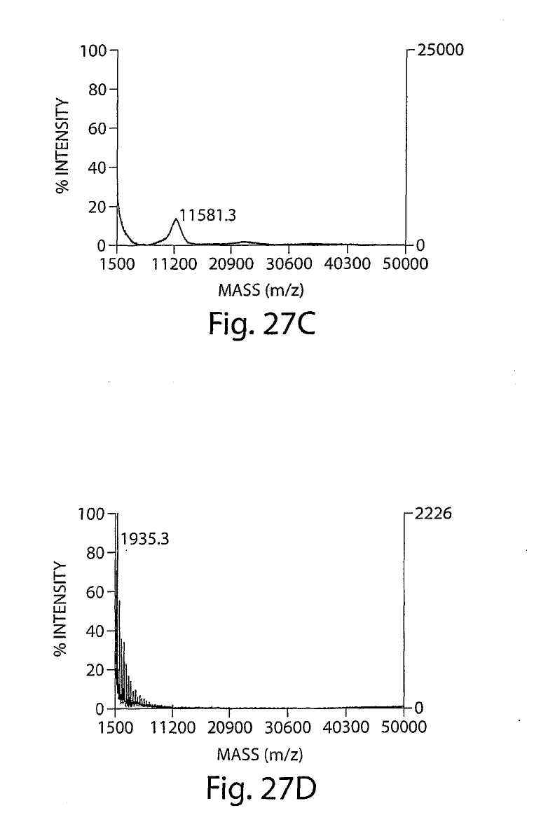

With the demonstrated reversal of labeling of RecA, the reaction of penicillamine with gold-bound metallothionein was examined. FIG. 27 shows the stability of gold bound to metallothionein after exposure to 20 mM penicillamine, which is much more than that needed to treat RecA. This concentration places penicillamine in a slight molar excess to cysteine at a ratio of 6 to 1. Samples were subjected to MALDI mass spectrometry to monitor the mass of gold-bound metallothionein. In all of the spectra, a slow decreasing ramp of signal is observed between 1500 amu to about 5000 amu. This is attributed to an increased detection of background noise possibly from increased sensitivity of the instrument at lower mass to charge values. Within most mass spectrometry experiments, this background noise would be removed during post-experimental processing of data. However, the wide distribution of gold-bound metallothionein peaks makes this baseline correction difficult. Therefore this step was eliminated. Panel A shows a negative control of apo-metallothionein. As witnessed in Chapter 2, a mass of 6137 amu is observed that can be account for by the expected mass of 6,125 amu. As a second control, aurothiomalate without protein was assayed to assure that gold polymers were not the source of observed peaks. In this spectrum, a series of narrow peaks below 7000 amu was detected above background. These sharp peaks increase with decreased mass and are believed to be polymers composed of different amounts of aurothiomalate and gold. Panels B and C display the spectra for gold-bound metallothionein samples incubated without and with 20 mM penicillamine, respectively. The gold-bound metallothionein peak in Panel B shows a peak at 12,134 amu. This corresponds to a high degree of gold binding with about 30 gold atoms bound at the peak. Also of interest is the series of fine peaks below 5000 amu most likely due to the presence of gold polymers in the sample. Panel C, which displays the result of penicillamine exposure, shows slight reduction of the gold-bound metallothionein peak to about 11,581 amu and a complete disappearance of the sharp gold polymer peaks. The slight mass reduction signifies about 27 gold atoms at the mass peak value. Although this shows a reduced value, it is interesting to note the gold-bound metallothionein appears fairly resistant to penicillamine especially as compared to the aurothiomalate polymers. This suggests that gold is bound extremely stably within metallothionein gold clusters.

### Example 5

### Purification of Metallothionein Fusion Proteins

Native metallothionein is rarely used in published research. Initial work on native, isolated metallothionein showed the identity of the bound metal and the metal content, but it was difficult to perform biochemical studies upon this protein [71] [90]. This was due to the presence of trace metals and a lack of homogeneity in the sample. Instead, metal-reconstituted metallothioneins are often utilized. Hence, non-native metallothionein purification techniques often involve the use of harsh treatments including boiling, strong acid treatments, and extremely high concentrations of reducing agents [90] [68]. These would be functionally deleterious to all but a few, if any, possible metallothionein fusion proteins. Therefore, less harsh methods were needed.

Reports of metal binding-directed purification procedures for metallothionein are rare. Purification of metallothionein from Arabidopsis thaliana that used copper and zinc charged affinity columns has been previously reported. However, like other procedures, this metallothionein was subsequently stripped of its metal during later purification steps and subjected to strong reducing agents. Furthermore, exact knowledge of the metal content of the final product is unknown [91]. With the known metal binding preferences of metallothionein for certain metals and with the assumption that metallothionein fusion proteins would contain zinc in their metal binding sites, attempts to purify metallothionein-containing proteins were undertaken.

In this Example, we describe development and utilization of a novel metal-based affinity method relying on metallothionein as a clonable purification tag. Specifically, zinc-bound metallothionein is demonstrated to bind to a cadmium-charged metal column. Furthermore, protein isolated in this method has undergone complete metal exchange with metal from the cadmium column.

### Materials and Methods

### Cloning of the Kinesin-Metallothionein-BCCP-5×His Construct

To construct the kinesin-metallothionein fusion protein containing a C-terminal hexa-histidine tag, a plasmid, pOU-I was obtained as gift from the Gelles laboratory at Brandeis University. This plasmid contains a dimeric Drosophila kinesin heavy chain gene, fused with a BCCP, biotinylatable domain sequence, and with a hexa-histidine sequence. The fused genes in this plasmid contained a unique NcoI site at the location corresponding to the kinesin F401-BCCP fusion. The metallothionein gene was produced by PCR using plasmid pET3-dMT. This plasmid containing the metallothionein gene was a gift from the Winge laboratory at the University of Utah. One primer, (5′-CTCGGGATCGAGGGAAGGATTTCAAGATATAC CATGGACCCC-3′) (SEQ ID NO: 2), codes for the beginning of the metallothionein sequence and contains a unique NcoI site. The second primer, (5′-GTGACCACATGTCACAGCACGT GCACTTGTCC-3′) (SEQ ID NO: 4), provides an AflIII site at the end of the sequence and removes metallothionein's stop codon. Plasmid pOU-I was prepared by restriction with NcoI, and the PCR product was prepared by restriction with NcoI and AflIII. Upon ligation of the two DNA fragments, the gene has a 50 percent chance of inserting in the correct orientation due to complimentary overhanging sequences. After transformation into Novablue cells (Novagen, Madison, Wis.), colonies were screened by restriction analysis for proper orientation, and likely constructs were sequenced for verification. The new construct was designated pOU-IMT.

### Nickel Column Purification of the Kinesin-Metallothionein-BCCP-5×His Construct

To express protein, pOU-IMT was transformed into BL21(DE3) cells (Novagen, Madison, Wis.). Single colonies were used to inoculate 10 mL LB starter cultures that were grown overnight at 30 degrees Celsius with aeration and selection. The next morning a 2 L LB culture was inoculated with the overnight growth and grown at 37 degrees Celsius with aeration and selection until an optical density at 600 nm of 0.5 was reached. At this point, IPTG was added to 0.2 mM, and the culture was supplemented with zinc sulfate to 0.2 mM. Cultures were shifted to room temperature and grown at least six hours to overnight with aeration. After this period, cells were pelleted at 6000×G and frozen with liquid nitrogen. Cells were stored at −70 degrees Celsius until the time of purification.

On the day of purification, cells were defrosted on ice and resuspended in 4 mL/gr Buffer A (20 mM imidazole buffer pH 7.2, 4 mM magnesiun chloride, 0.9 mM 2-mercaptoethanol, and EDTA-free Complete® protease inhibitor cocktail (Roche, Indianapolis, Ind.)). To this, lysozyme, DNAseI, and RNAse A were added to 1 mg/mL, 0.5 mg/mL, and 1 mg/mL, respectively. Cells were incubated on ice for 0.5 hours. Following three rounds of freezing in liquid nitrogen and thawing on ice, cells were completely lysed. The cell suspension was spun at 9000×G for 30 minutes. The clarified supernatant was removed and diluted to 100 mL with Buffer A that was supplemented with 50 μM ATP. The mixture was loaded onto a Pharmacia Fast Flow nickel-charged IDA column at 0.5 mL/min. Once loaded, the column was washed with Buffer A that was supplemented with 50 μM ATP until the OD at 280 nm returned to baseline. Elution was performed with Buffer B (500 mM imidazole buffer pH 7.2, 4 mM magnesium chloride, 0.9 mM 2-mercaptoethanol, and EDTA-free Complete® protease inhibitor cocktail) in 1 mL fractions. Protein was verified by SDS-PAGE and western blots using anti-His tag antibody (Invitrogen, Carlsbad, Calif.).

### Assay for Metallothionein Binding to Immobilized Metal Columns

To assess which type of metal charge column is able to bind zinc-bound metallothionein, four 1 mL Pharmacia (Amersham Biosciences, Piscataway, N.J.) Fast Flow NTA columns were utilized. Three columns were charged with nickel, zinc, or cadmium as described in the manufacturer's directions. The fourth column was prepared with no metal as a negative control. In all steps, a 20 mM Tris-HCl pH 7.5 buffer was used. After addition of metal, columns were washed with 10 mL of buffer. Then, 0.1 mL of a 2 mg/mL zinc metallothionein (M9542, Sigma Chemical Company, St. Louis, Mo.) solution was added to each column. Immediately, 0.4 mL of buffer was added for a total of 0.5 mL that was collected in the first fraction. Next each column was washed with 1.5 mL of buffer monitoring the elution in 0.5 mL fractions. Finally, bound protein was eluted with buffer supplemented with 50 mM EDTA while collecting 0.5 mL fractions. All samples were subjected to SDS-PAGE to evaluate column binding.

### ESI Mass Spectrometry

For ESI mass spectrometry, samples were desalted by buffer exchange in a spin concentration device. First, the sample was concentrated to 50 uL by spinning the sample within a YM-3 Centricon (Millipore, Billerica, Mass.) at 12000×G in a tabletop microfuge. Desalting was performed by repeated dilution to 500 uL in 25 mM Tris-HCl followed by re-concentration to 50 uL in the YM-3 microcentricon for two times. This was repeated 3 more times using a suitable ESI mass spectrometry buffer. For these experiments, the buffer was a 5 mM ammonium acetate buffer pH 6.0. Finally, the sample was diluted to 100 uL with 5 mM ammonium acetate buffer pH 6.0 with 40% methanol to give a final concentration of 20% methanol in the sample. For an apo-metallothionein control, a small volume (less than 5 μL) of 0.1 M acetic acid was added to a Zn₇-metallothionein sample to lower the pH below pH 4 and cause release of zinc from the protein. All samples were spun through a 0.4 μm spin filter (Amicon).

All ESI mass spectrometry samples were run at the Brandeis University Biochemistry Core Facility on a Perseptive Biosystems Mariner (Framingham, Mass.). Samples were typically diluted an additional 5 or 20 fold at the time of recording in a 5 mM ammonium acetate buffer pH 6.0 with 20% methanol to obtain a strong signal with minimal protein amount. Samples were injected at a rate of 3-5 uL/minute and collected over a mass to charge range of 500 to 4000. Nozzle potential and detector potential were adjusted to obtain strong clean spectra. All samples were collected in positive ion mode. The instrument calibration was verified on the day of the experiment using apo-myoglobin (A8673, Sigma Chemical Corporation) in a 5 mM ammonium acetate buffer pH 6.0 with 20% methanol.

### Construction of the Fimbrin N375 Metallothionein Fusion

The n-terminal 375 amino acid sequence of human fimbrin, which contains only one actin-binding domain, was fused to the mouse metallothionein-I gene with a short Ser-Gly-Ser-Gly linker. Plasmid pAB-4×, containing, the fimbrin gene, was provided by the Matsudaira laboratory (Whitehead Institute). Metallothionein was provided as plasmid pET3-dMT from the Winge laboratory (University of Utah). The fimbrin gene was amplified by PCR. The first primer, (5′-CGCCCTCTAGAAATAATTTTGTTTAACTTTAAGAAGGAGATATACATA T GGATGAGATGGCTACCACTC-3′) (SEQ ID NO: 5), contains a unique XbaI site located within a T7 promotor sequence and a start methionine coded within an NdeI restriction site. The second primer, (5′-GCCGGATCCCTAAACCATGGCACCCGATCCAGAATTAAACAGGTT AGCCACGAAAGC-3′) (SEQ ID NO: 6), contains the linker region along with a unique NcoI site. This PCR product and pET3-dMT were prepared by restriction with NcoI and XbaI, and subsequently they were ligated together. The new plasmid, designated pET3-aFimMT, contains the gene fusion in a plasmid resembling the commercial pET3-a vector (Novagen, Madison, Wis.).

### Purification of the Fimbrin N375 Metallothionein Fusion

For expression of the fimbrin N375 metallothionein fusion protein, pET3-aFimMT was transformed into BL21 (E3) cells (Novagen, Madison Wis.). Single colonies were used to inoculate 10 mL LB starter cultures that were grown overnight at 30 degrees Celsius with aeration and selection. The next morning a 1 L LB culture was inoculated and grown at 37 degrees Celsius with aeration and selection until an optical density at 600 nm of 0.5 was reached. At this point, IPTG was added to 0.2 mM. After 0.5 hours, the culture was supplemented with zinc sulfate to 0.2 mM and grown an additional 1.5 hours. Cells were then harvested by centrifugation at 6000×g for 20 minutes. The cell pellet was frozen in liquid nitrogen and stored at −70 degrees Celsius until the day of purification.

On the day of purification, the cell pellet was defrosted on ice. Once defrosted, the pellet was resuspended in 15 mL Wash Buffer (25 mM Tris-Base Buffer pH 7.5, 150 mM sodium chloride, and 0.1 mM 2-mercaptoethanol). Suspensions were sonicated 8 times with 30 second pulses and 1 minute rest periods on ice with a Branson 2000 sonicator to lyse cells. Lysis was monitored with Biorad Total Protein Concentration (Hercules, Calif.) solution.

Once lysed, cell suspensions were spun at 9000×g. The resulting supernatant was supplemented with zinc sulfate to 0.2 mM and mixed. The supernatant was then loaded onto a 200 mL Sephadex G100 column (Amersham Biosciences, Piscataway, N.J.) to desalt the protein. The column was run by gravity with Wash Buffer while collecting 8 mL fractions. Fractions were monitored using SDS-PAGE. Furthermore, fractions containing the desired protein were combined and loaded onto a pre-charged 1 mL Pharmacia (Amersham Biosciences, Piscataway, N.J.) Fast Flow cadmium-charged NTA column at 0.5 mL/min. The column was then washed with 5 column volumes of Wash buffer. Finally, column bound protein was eluted using Wash Buffer supplemented with 50 mM EDTA while collecting 0.5 mL fractions.

### Results

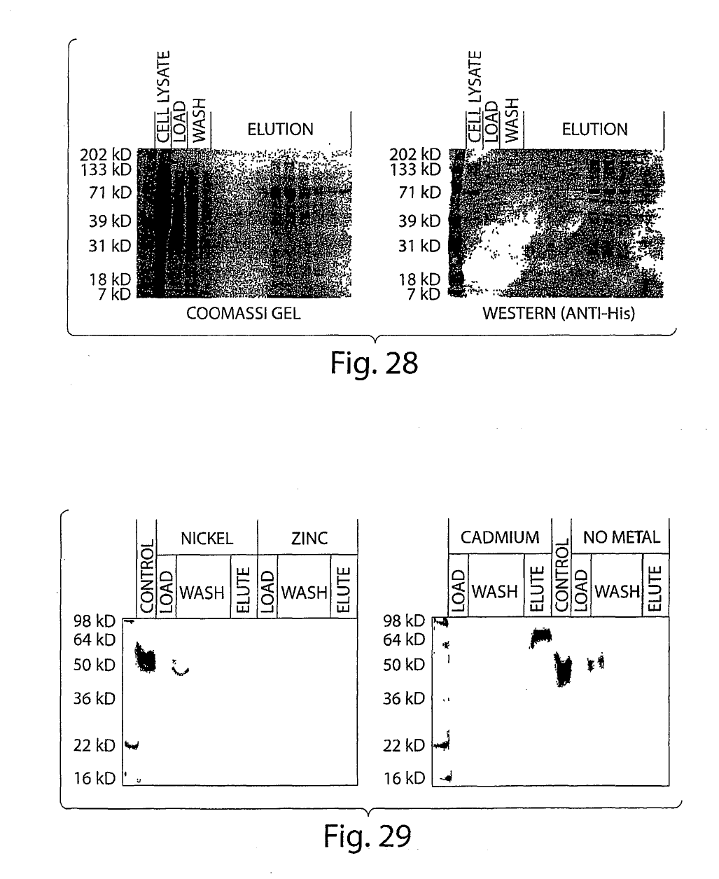

FIG. 28 shows the results of a purification of the kinesin-metallothionein construct. This hexa-histidine tagged protein is affinity purified by the nickel column. However, the multiple bands in the gels show that the protein may require additional purification steps. The western shows that some degree of proteolysis is occurring. Since the antibody for this western is directed to the hexa-histidine sequence, only fragments containing this sequence are identified. Therefore, it is possible that several of the other bands witnessed in the Coomassie gel can be attributed to other proteolytic fragments not recognized in the western. Interestingly, initial purification attempts using Tris and PIPES buffering systems did not result in protein binding to the metal charged column. Only upon use of a low-level imidazole buffer was metal affinity purification possible. This may suggest interference of metallothionein with the hexa-histidine sequence. Perhaps this histidine tag, in the absence of competing imidazole, coordinates with metal atoms bound to metallothionein. The low-level imidazole buffer may free the histidines tag so that it can coordinate to column bound metal atoms.

A reverse engineering approach was taken to determine whether affinity purification of metallothionein containing fusion proteins using metallothionein's metal binding ability was possible. For this, commercially available zinc-bound metallothionein was first evaluated for its ability to bind immobilized metal columns charged with various metals. Columns able to bind the protein were identified. Then, columns containing bound protein were evaluated for compounds able to cause protein removal. FIG. 29 shows the results of metallothionein passed through four different metal-charged IDA columns. Columns charged with nickel, zinc, or no metal show zinc-bound metallothionein passing through each column without binding. The protein can be found in the first wash fraction in each case. However, the cadmium-charged column shows the protein is retained in the column and only removed upon treatment with EDTA. Here, the protein did not elute until the second EDTA fraction. Of the compounds tested for elution from the cadmium column, only the strong chelating compounds, EDTA and EGTA, resulted in elution (not shown). In the gels shown in FIG. 29, metallothionein's mobility is shown as an elongated smear at about 50 kD for all conditions except for the cadmium elution fraction that travels at about 80 kD. These are far greater than the expected mass of about 6 kD. This altered mobility is a common feature witnessed with metallothionein proteins [92]. This shift may suggest that metallothionein may still have metal bound even after SDS treatment and boiling.

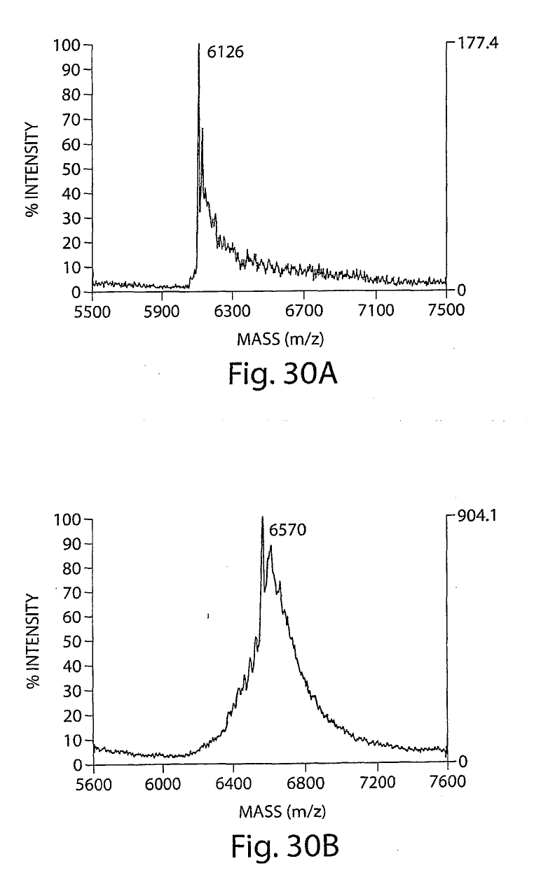

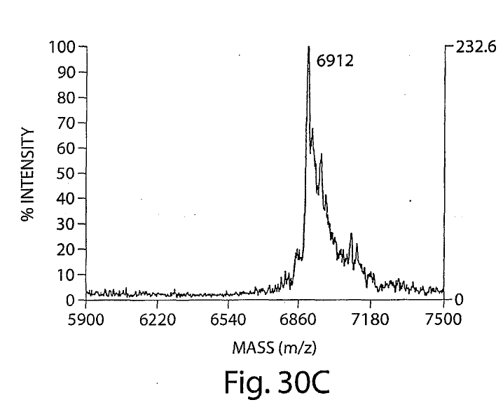

One surprise of this work was that metallothionein eluted from the cadmium-charged IDA columns with EDTA contained bound metal atoms. The strong chelating agent is unable to remove the metal from the protein binding sites. FIG. 30 shows ESI mass spectrometry spectra. Panels A and B are the deconvoluted spectra from two control samples, apo-metallothionein and zinc-bound metallothionein, respectively. Apo-metallothionein has an expected mass of 6125 amu. The apo-metallothionein spectrum agree extremely well with this expected value and the previously reported experimental value of 6126 amu [63]. Panel B, displaying the zinc form of the protein, shows a peak at 6570 amu. Again, this agrees well with the expected mass of 6569 amu and the observed mass of 6571 amu reported previously [63]. Panel C displays the deconvoluted spectrum for the protein eluted from the cadmium-charged column. This spectrum shows a peak at 6912 amu. A metallothionein protein with 7 cadmium atoms bound has an expected mass of 6898 amu. The mass witnessed here is in good agreement with this value. Hence, this observed mass suggests that a complete metal exchange of the 7 zinc atoms for 7 cadmium atoms occurs upon elution from the column.

After determining that zinc-bound metallothionein can bind to a cadmium column and can be subsequently removed, attempts to purify a fimbrinN375-metallothionein fusion protein were made. Initial attempts without supplementation of cell lysates with zinc and without a desalting step, resulted in the stripping of cadmium from the IDA column. Moreover, the column flow through from the load fraction developed into a milky white colloid shortly after leaving the column. Addition of EDTA to this fraction resulted in a change of the sample back to a clear solution. This dispersion and the ability to strip cadmium from the column suggests the colloid was possibly formed by cadmium saturated metallothionein. A second consequence of these observations is that it shows the possible importance of zinc binding for proper isolation using this method. The MBP-MT data of Example 2 showed limited metal binding within metallothionein fusion proteins isolated from cells. This observation may explain the reason for the fusion protein to strip cadmium from the column and for the milky colloid development. Perhaps zinc is needed to act as a counter balance for proper cadmium binding. As apo-metallothionein travels through the cadmium column, it may try to accommodate as many metal atoms as possible. Zinc may prevent metallothionein from completely unfolding as it passes through the column, thus limiting the proteins reactivity. With this observation, the zinc supplement and desalting steps were added to the purification procedure.

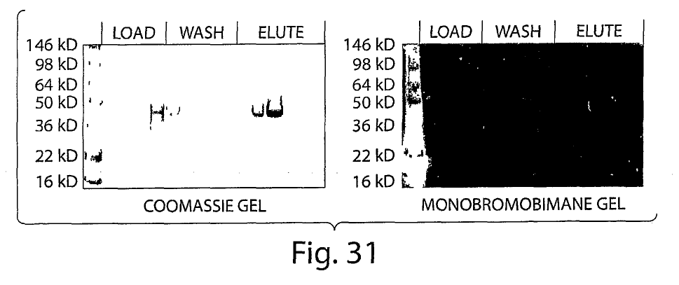

FIG. 31 displays an SDS-PAGE gel of protein from the cadmium-bound column purification of fimbrinN375-metallothionein. The gel was stained with Coomassie and monobromobimane to evaluate fractions. Monobromobimane labels cysteines with a fluorescent moiety that allows for visualization of cysteine-rich proteins upon UV excitation. This chemical is commonly used for tracking metallothionein [91]. These gels shows that the method developed here can provide a clean affinity purification of metallothionein fusion proteins.

The practice of the present invention may employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

### REFERENCES

1. DeRosier, D. J. and A. Klug, Reconstruction of three-dimensional structures from electron micrographs. Nature, 1968. 217: p. 130-134.
2. Henderson, R., et al., A model for the structure of bacteriorhodopsin based on high resolution electron cryomicroscopy. J Mol Biol, 1990. 213: p. 899-929.
3. Grigorieff, N., et al., Electron-crystallographic refinement of the structure of bacteriorhodopsin. J Mol Biol, 1996. 259: p. 393-421.
4. Koster, A. J., et al., Perspectives of molecular and cellular electron tomography. J Struct Biol, 1997. 120(3): p. 276-308.
5. McIntosh, J. R., Electron microscopy of cells: a new beginning for a new century. J Cell Biol, 2001. 153(6): p. F25-32.
6. Sjostrand, F. S., Electron Microscopy of Cells and Tissue. 4 ed. 1967, New York: Acedemic Press, Inc. 462.
7. Ceska, T. A., Recollections of the Electron Crystallographic Heavy Atom Derivative. Journal of Structural Biology, 1999. 127: p. 135-140.
8. Powell, R. D., et al., Giant platinum clusters: 2-nm covalent metal cluster labels. J Struct Biol, 1999. 127(2): p. 177-84.
9. Safer, D., et al., Biospecific labeling with undecagold: visualization of the biotin-binding site on avidin. Science, 1982. 218(4569): p. 290-1.
10. Safer, D., L. Bolinger, and J. S. Leigh, Jr., Undecagold clusters for site-specific labeling of biological macromolecules: simplified preparation and model applications. J Inorg Biochem, 1986. 26(2): p. 77-91.
11. Jahn, W., Review: chemical aspects of the use of gold clusters in structural biology. J Struct Biol, 1999. 127(2): p. 106-12.
12. Wenzel, T. and W. Baumeister, Conformational constraints in protein degradation by the 20S proteosome. Nat. Struct Biol, 1995. 2: p. 199-204.
13. Gregori, L., et al., Amyloid beta-protein inhibits ubiquitin-dependent protein degradation in vitro. J. Biol. Chem., 1997. 272: p. 58-62.
14. Weinstein, S., et al., Metal Compounds as Tools for the Construction and the interpretation of Medium-Resolution Maps of Ribosomal Particles. Journal of Structural Biology, 1999. 127: p. 141-151.
15. Adams, S. R., et al., New biarsenical ligands and tetracysteine motifs for protein labeling in vitro and in vivo: synthesis and biological applications. J Am Chem Soc, 2002. 124(21): p. 6063-76.
16. Gaietta, G., et al., Multicolor and electron microscopic imaging of connexin trafficking. Science, 2002. 296(5567): p. 503-7.
17. Deerinck, T. J., et al., Fluorescence photooxidation with eosin: a method for high resolution immunolocalization and in situ hybridization detection for light and electron microscopy. J Cell Biol, 1994. 126(4): p. 901-10.
18. Safer, D., Undecagold cluster labeling of proteins at reactive cysteine residues. J Struct Biol, 1999. 127(2): p. 101-5.
19. Hainfeld, J. F., W. Liu, and M. Barcena, Gold-ATP. J Struct Biol, 1999. 127(2): p. 120-34.
20. Hainfeld, J. F., et al., Ni-NTA-gold clusters target His-tagged proteins. J Struct Biol, 1999. 127(2): p. 185-98.
21. Griffiths, G., Fine Structure Immunocytochemistry. 1993, Heidelberg: Springer Verlag.
22. Schaaff, T. G., et al., Isolation and Select Properties of a 10.4 kDa Gold: Glutathione Cluster Compound. J Phys. Chem. B., 1998. 102(52): p. 10643-10646.
23. Gutierrez, E., et al., Greengold, a giant cluster compound of unusual electronic structure. Eur. Phys. J. D, 1999. 9: p. 647-651.
24. Mingos, M. P., Bonding in Molecular Clusters and Their Relationship to Bulk Metals. Chem. Soc. Rev., 1986.15: p. 31-61.
25. Brown, L. O. and J. E. Hutchison, Convenient preparation of stable, narrow-dispersity, gold nanocrystals by ligand exchange reactions. J. Am. Chem. Soc., 1997. 119: p. 12384-12385.
26. Schaaff, T. G. and R. L. Whetten, Giant Gold-Glutathione Cluster Compounds: Intense Optical Activty in Metal-Based Transitions. J Phys. Chem. B., 2000.104: p. 2630-2641.
27. Rauser, W. E., Phytochelatins and related peptides. Structure, biosynthesis, and function. Plant Physiol, 1995. 109(4): p. 1141-9.
28. Sano, T., A. N. Glazer, and C. R. Cantor, A streptavidin-metallothionein chimera that allows specific labeling of biological materials with many different heavy metal ions. Proc Natl Acad Sci USA, 1992. 89(5): p. 1534-8.
29. Sawyer, J. R., P. W. Tucker, and F. R. Blattner, Metal-binding chimeric antibodies expressed in Escherichia coli. Proc Natl Acad Sci USA, 1992. 89(20): p. 9754-8.
30. M Margoshes, B. V., Journal of the American Chemical Society, 1957. 79: p. 4813-4814.
31. J Kagi, T. C., J Ovemell, M Webb, Synthesis and Function of Metallothioniens. Nature, 1981.292: p. 495-496.
32. Hamer, D. H., Metallothionein. Annu Rev Biochem, 1986. 55: p. 913-51.
33. Welch, J., et al., The CUP2 gene product regulates the expression of the CUP1 gene, coding for yeast metallothionein. Embo J, 1989. 8(1): p. 255-60.
34. Binz, P. A. and J. H. R. Kagi, Molecular Evolution of Metallothioneins: Contributions from coding and non-coding Regions. 1999, www.unizh.ch/˜mtpage/poster/posterevol.html.
35. Higham, D., P. Sadler, and M. Scawen, Cadmium-resistant Pseudomonas putida Synthesizes Novel Cadmium Proteins. Science, 1984. 225: p. 1043-1046.
36. Silver, S., Bacterial resistance to toxic metal ions—a review. Gene, 1996. 179: p. 9-19.
37. Robbins, H. A., et al., Redefined crystal structure of Cd,Zn metallothionein at 2.0 Å resolution. J Mol Biol, 1991. 221: p. 1269-1293.
38. Pande, J., M. Va\check{s}ák, and J. H. R. Kägi, Interaction of Lysine Residues with the Metal Thiolate Clusters in Metallothionein. Biochemistry, 1985. 24: p. 6717-6722.
39. Furey, W. F., et al., Crystal Structure of Cd,Zn Metalllothionein. Science, 1985. 231: p. 704-710.
40. Rhee, I. K., K. S. Lee, and P. C. Huang, Metallothioneins with interdomain hinges expanded by insertion mutagenisis. Protein Engineering, 1990. 3: p. 205-213.
41. Kurasaki, M., et al., Protein Engineering, 1996. 9: p. 1173-1180.
42. Kurasaki, M., et al., Significance of alpha-fragment of metallothionein in cadmium binding. Protein Engineering, 1997. 10(4): p. 413-416.
43. Nielson, K. B., C. L. Atkin, and D. R. Winge, Distinct metal-binding configurations in metallothionein. J Biol Chem, 1985. 260(9): p. 5342-50.
44. Hou, T., et al., Cysteine-independent polymerization of metallothioneins in solutions and in crystals. Protein Sci, 2000. 9(12): p. 2302-12.
45. Jiang, L. J., W. Maret, and B. L. Vallee, The ATP-metallothionein complex. Proc Natl Acad Sci USA, 1998. 95(16): p. 9146-9.
46. Maret, W. and B. L. Vallee, Thiolate ligands in metallothionein confer redox activity on zinc clusters. Proc Natl Acad Sci USA, 1998. 95(7): p. 3478-82.
47. Zangger, K., G. Oz, and I. M. Armitage, Re-evaluation of the binding of ATP to metallothionein. J Biol Chem, 2000. 275(11): p. 7534-8.
48. Good, M., et al., 113Cd NMR studies on metal-thiolate cluster formation in rabbit Cd(II) metallothionein: evidence for pH dependence. Biochemistry, 1988. 27(18): p. 7163-7166.
49. Ejinik, J., et al., Folding pathway of apo-metallothionein induced bu Zn²⁺, Cd²⁺, and Co²⁺. J Inorg. Biochem., 2002. 88: p. 144-152.
50. Mogilnicka, E. M. and J. K. Piotrowski, Inducible gold-binding proteins in rat kidneys. Biochem Pharmacol, 1979. 28(17): p. 2625-31.
51. Mogilnicka, E. M. and M. Webb, Time-dependent uptake and metallothionein-binding of gold, copper and zinc in the rat kidney. Biochem Pharmacol, 1983. 32(8): p. 1341-6.
52. Laib, J. E., et al., Formation and characterization of aurothioneins: Au,Zn,Cd-thionein, Au,Cd-thionein, and (thiomalato-Au)chi-thionein. Biochemistry, 1985. 24(8): p. 1977-86.
53. Shaw III, C. F., et al., Biphasic Kinetics of Aurothionein Formation from Gold Sodium Thiomalate. Inorg. Chem., 1990. 29: p. 403-408.
54. Gasyna, Z., et al., Inorg. Chim. Acta., 1988. 153(115-118).
55. Li, H. and J. D. Otvos, HPLC Characterization of Ag+ and Cu+ Metal Exchange Reactons with Zn- and Cd-Metallothioneins. Biochemistry, 1996. 35: p. 13937-13945.

56. Lu, W., A. Zelazowski, and M. J. Stillman, Mercury Binding to metallothionein: Formation of the Hg18-MT Species. Inorg. Chem., 1993. 32: p. 919-926.
57. Jiang, D. T., et al., Structures of the Cadmium, Mercury, and Zinc Thiolate Clusters in Metallothionein. J. Am. Chem. Soc., 1994. 116: p. 11004-11013.
58. Mogilnicka, E. M. and J. K. Piotrowski, Binding of gold in the kidney of the rat. Biochemical Pharmacology, 1980. 26: p. 1819-1820.
59. Sharma, R. P. and E. G. McQueen, The binding of gold to cytosolic proteins of the rat liver and kidney tissues: metallothioneins. Biochem Pharmacol, 1980. 29(14): p. 2017-21.
60. Schmitz, G., et al., The binding of Gold(I) to metallothionein. J Inorg Biochem, 1980. 12(4): p. 293-306.
61. Gehrig, P. M., et al., Electrospray ionization mass spectrometry of zinc, cadmium, and copper metallothioneins: evidence for metal-binding cooperativity. Protein Sci, 2000. 9(2): p. 395-402.
62. Schaaff, T. G., et al., Properties of a Ubiquitous 29 kDa Au:SR Cluster Compound. J. Phys. Chem. B., 2001. 105(37): p. 8785-8796.
63. Yu, X., M. Wojciechowski, and C. Fenselau, Assessment of Metal in Reconstituted Metallothioneins by Electrospray Mass Spectrometry. Anal. Chem., 1993.65: p. 1355-1359.
64. Schaaff, T. G., et al., Isolation of Smaller Nanocrystal Au Molecules: Robust Quantum Effects in Optical Spectra. J. Phys. Chem. B., 1997. 101(7885-7891).
65. Alvarez, M. M., et al., Critical sizes in the growth of Au clusters. Chemical Physics Letters, 1997. 266: p. 91-98.
66. Shaw III, C. F., J. Eldridge, and M. P. Cancro, 13C NMR Studies of Aurothioglucose Ligand Exchange and Redox Disproportionation Reaction. Journal of Inorganic Biochemistry, 1981. 14: p. 267-274.
67. Saito, S. and M. Kurasaki, Gold replacement of cadmium, zinc-binding metallothionein. Res Commun Mol Pathol Pharmacol, 1996. 93(1): p. 101-7.
68. Vasak, M., Metal Removal and Substitution in Vertebrate and Invertebrate Metallothioneins, in Methods in Enzymology. 1991, Academic Press. p. 452-458.
69. Hall, C., Introduction to Electron Microscopy. 2nd ed. International Series in Pure and Applied Physics, ed. L. Schiff. 1966, New York: McGraw-Hill inc.
70. Egerton, R. F., Electron Energy-Loss Spectroscopy in the Electron Microscope. 2nd ed. 1996, New York, N.Y.: Plenum Press. 485.
71. Suzuki, K., Purification of Vertebrate Metallothioneins, in Methods in Enzymology. 1991, Acedemic Press. p. 252-263.
72. Hou, Y. M., R. Kim, and S. H. Kim, Expression of the mouse metallothionein-I gene in Escherichia coli: increased tolerance to heavy metals. Biochim Biophys Acta, 1988. 951(1): p. 230-4.
73. DeRosier, D., The Reconstruction of Three-Dimensional Images from Electron Micrographs. Contemp. Phys., 1971. 12(5): p. 437-452.
74. Wenzel, T. and W. Baumeister, Conformational constraints in protein degradation by the 20S proteasome. Nat. Struct Biol, 1995. 2: p. 199-204.
75. Dubochet, J., et al., Cryo-electron Microscopy of Vitrified Specimens. Q Rev Biophys, 1988. 21(2): p. 128-228.
76. Sabil, H. R., Conformational changes studied by cryo-electron microscopy. Nat Struct Biol, 2000. 7(9): p. 711-714.
77. Sandin, S., et al., Structure and Flexibility of Individual Immunoglobulin G Molecules in Solution. Structure, 2004. 12: p. 409-415.
78. Saphire, E. O., et al., Contrasting IgG structures reveals extreme asymmetry and flexibility. J Mol Biol, 2002. 319: p. 9-18.
79. Fricker, S., Medicinal chemistry of gold compounds, in The Chemistry of Organic Derivatives of GOld and Silver, S. Patai and Z. Rappaport, Editors. 1999, John Wiley & Sons Ltd.: New York. p. 641-657.
80. Shaw III, C. F., The Biochemistry of Gold. Inorg. Persp. Biol. Med., 1979. 2(278): p. 287-355.
81. Isab, A. A. and P. J. Sadler, A Carbon-13 Nuclear Magnetic Resonance Study of Thiol Exchange of Gold(I) Thiomalate including Applications to Cysteine Derivatives. J. C. S. Dalton, 1982: p. 135-141.
82. Karlin, S. and L. Brocchieri, Evolutionary conservation of RecA genes in relation to protein structure and function. J Bacteriol, 1996. 178(7): p. 1881-94.
83. Kowalczykowski, S. C., et al., Biochemistry of homologous recombination in E. Coli. Microbiological Reviews. Vol. 58. 1994.401-465.
84. Weisemann, J. and G. Weinstock, Mutations at the cysteine codons of the recA gene of Escherichia coli. DNA, 1988. 7(6): p. 389-398.
85. Kuramitsu, S., et al., Cysteinyl residues of Escherichia coli recA protein. Biochemistry, 1984. 23(11): p. 2363-2367.
86. Schaeffer, N., et al., In vitro penicillamine competition of protein-bound gold(I). Arthrisis Rheum, 1980. 23(2): p. 165-171.
87. Sheibani, N., Prokaryotic gene fusion expression systems and their use in structural and functional studies of proteins. Preparative Biochemistry and Biotechnology, 1999. 29(1): p. 77-70.
88. Sousa, H. and R. A. Milligan, Three-dimensional Structure of ncd-decorated Microtubules Obtained by a Back-projection Method. J Mol Biol, 1996. 260: p. 743-755.
89. Gelles, J. 2003 personal communication.
90. Vasak, M., Large-scale preparation of metallothionein: biological sources. Methods Enzymol, 1991. 205: p. 39-41.
91. Murphy, A., et al., Purification and immunological identification of metallothioneins 1 and 2from Arabidopsis thaliana. Plant Physiol, 1997. 113(4): p. 1293-301.
92. Hidalgo, J., et al., Effects of 2-mercatpoethanol on the electrophoretic behavior of rat and dogfish metallothionein and chromatographic evidence of a naturally occurring metallothionein polymerization. Comp. Biochem. Physiol. C, 1988. 89(2): p. 191-196.
93. Pearson, R. G., Hard and Soft Acids and Bases. Benchmark papers in Inorganic Chemistry, ed. R. G. Pearson. 1973, Stroudsburg, Pa.: Dowden, Hutchinson, and Ross.
94. Schmid et al., Large transition metal Polyhedron, 1988. 7: p. 605-608.
95. Frey et al., Synthesis of undecagold Journal of Structural Biology, 1999 p. 94-100.

### INCORPORATION BY REFERENCE

All publications, patents and sequence database entries mentioned herein are hereby incorporated by reference in their entirety as if each individual publication or patent was specifically and individually indicated to be incorporated by reference. Also incorporated by reference are the following: dissertation by Christopher Mercogliano entitled Development of a Clonable Transmission Electron Microscopy Label (Brandeis University 2004); and Mercogliano and Derosier, J. Mol. Biol. 355: 211-223 (2006) (epub November 2005). In case of conflict, the present application, including any definitions herein, will control.

While specific embodiments of the subject inventions are explicitly disclosed herein, the above specification is illustrative and not restrictive. Many variations of the inventions will become apparent to those skilled in the art upon review of this specification and the claims below. The full scope of the inventions should be determined by reference to the claims, along with their full scope of equivalents, and the specification, along with such variations.

1. A method for analyzing a target protein by electron microscopy, comprising:
a) providing a sample comprising a fusion protein comprising the target protein and metallothionein or a fragment thereof;
b) contacting the sample with a source of heavy atoms; and
c) analyzing the sample by electron microscopy.

2. The method of claim 1, wherein the fusion protein is present in a cell.

3. The method of claim 2, further comprising introducing a nucleic acid construct comprising a nucleotide sequence encoding the fusion protein into the cell under conditions that permit expression of the fusion protein.

4. The method of claim 3, wherein the nucleic acid construct is contained in an expression vector.

5. The method of claim 3, wherein the nucleic acid construct is capable of integrating into the genome of the cell.

6. The method of claim 1, further comprising fixing the sample prior to analysis by electron microscopy.

7. The method of claim 6, wherein the sample is fixed by at least one of the following: chemical fixation, embedding, or freezing.

8. The method of claim 6, further comprising slicing the sample into thin sections of a thickness in the range of about 25 nm to 1 μM.

9. The method of claim 1, wherein the heavy atoms are at least one of the following: gold (Au), Silver (Ag), mercury (Hg), cadmium (Cd), zinc (Zn), platinum (Pt), or bismuth (Bi).

10. The method of claim 9, wherein the heavy atoms are gold (Au).

11. The method of claim 10, wherein the source of gold is at least one of the following: aurothiomalate, aurothioglucose, or auranofin.

12. The method of claim 10, further comprising enhancing the gold label using silver precipitation.

13. The method of claim 2, further comprising modifying the cell to facilitate uptake of the source of heavy atoms by introducing into the cell a nucleic acid construct encoding one or more proteins from the mer operon of E. coli.

14. The method of claim 2, further comprising permeabilizing the cell membrane to facilitate uptake of the source of heavy atoms.

15. The method of claim 14, wherein the cell membrane is permeabilized by at least one of the following methods: contacting the cell with a detergent or electroporation.

16. The method of claim 2, wherein the cell is eukaryotic cell.

17. The method of claim 16, wherein the cell is contacted with a source of heavy atoms that is taken up by the cell.

18. The method of claim 17, wherein the source of heavy atoms is at least one of the following: aurothiomalate, aurothioglucose, or auranofin.

19. The method of claim 1, wherein the sample is analyzed using scanning electron microscopy (SEM) or transmission electron microscopy (TEM).

20. The method of claim 1, wherein the fusion protein comprises the target gene and at least two copies of metallothionein.

21. The method of claim 20, wherein the fusion protein comprises the target gene and at least three copies of metallothionein.

22. The method of claim 1, wherein the fusion protein comprises a fragment of metallothionein.

23. The method of claim 22, wherein the fragment of metallothionein comprises the alpha domain.

24. The method of claim 23, wherein the fusion protein comprises at least two copies of the alpha domain of metallothionein.

25. A method for analyzing a target protein in a eukaryotic cell by electron microscopy, comprising:
a) introducing a nucleic acid encoding a fusion protein comprising the target protein and metallothionein into the cell under conditions suitable for expression of the fusion protein;
b) contacting the cell with at least one of the following: aurothiomalate, aurothioglucose, or auranofin; and
c) analyzing the sample by electron microscopy.

26. A method for purifying a target protein comprising:
a) expressing a fusion protein comprising the target protein and metallothionein or a fragment thereof;
b) passing a sample comprising the fusion protein over a cadmium-charged (Cd) immobilized metal affinity chromatography (IMAC) column under conditions that permit association between metallothionein and the cadmium charged column; and
c) eluting the fusion protein from the column, thereby purifying the target protein.

27. The method of claim 26, wherein the column is washed prior to elution of the fusion protein.

28. The method of claim 26, wherein the column is eluted with EDTA or EGTA.

29. The method of claim 26, wherein the metallothionein is bound to one or more metal atoms prior to passing the fusion protein over the cadmium charged column.

30. The method of claim 29, wherein the metallothionein is bound to one or more of the following: zinc (Zn), gold (Au), Silver (Ag), mercury (Hg), cadmium (Cd), zinc (Zn), platinum (Pt), or bismuth (Bi).
